(19) United States
(12) Patent Application Publication
(10) Pub. No.: US 2025/0146007 A1
(43) Pub. Date: May 8, 2025

(54) METHODS TO PRODUCE RECOMBINANT CUTIBACTERIUM ACNES AND USES THEREOF

(71) Applicant: UNIVERSITAT POMPEU FABRA, Barcelona (ES)

(72) Inventors: Marc GÜELL CARGOL, Barcelona (ES); Nastassia KNÖDLSEDER, Barcelona (ES); María José FÁBREGA FERNÁNDEZ, Barcelona (ES); Javier SANTOS MORENO, Barcelona (ES); Guillermo NEVOT SÁNCHEZ, Barcelona (ES)

(21) Appl. No.: 18/832,444

(22) PCT Filed: Jan. 24, 2023
(86) PCT No.: PCT/EP2023/051708

§ 371 Date: Jul. 23, 2024

(30) Foreign Application Priority Data
Jan. 24, 2022 (EP) 22382050.7

(51) Int. Cl. C12N 15/74; A61K 8/99; A61K 35/00; A61K 35/74; A61Q 19/08; C07K 14/47

(52) U.S. Cl. CPC C12N 15/74; A61K 8/99; A61K 35/74; A61Q 19/08; C07K 14/47; A61K 2035/11

(57) ABSTRACT

The present invention relates to recombinant C. acnes and method of producing thereof. The present invention provides a human neutrophil gelatinase-associated lipocalin (NGAL) protein derived from a recombinant bacterium and its uses thereof. Particularly, the present invention relates to the use of NGAL protein obtained from a recombinant bacterium in the treatment of acne vulgaris or as a cosmetical composition. Provided herein are also recombinant bacterium expressing NGAL protein and their uses.

### TECHNICAL FIELD OF THE INVENTION

The present invention relates to recombinant C. acnes and method of producing thereof. The present invention relates to the field of dermatologic disorders. In particular, the present invention relates to use of neutrophil gelatinase-associated lipocalin (NGAL) protein produced by a bacterium in the treatment of retinoid-responsive conditions, particular acne vulgaris, in a subject in need thereof.

### BACKGROUND OF THE INVENTION

Retinoids (RA) have been used as therapeutic agents for numerous skin diseases, from psoriasis to acne and wrinkles. Acne vulgaris is a common chronic skin disease involving blockage and/or inflammation of pilosebaceous units (hair follicles and their accompanying sebaceous gland). Acne can present as noninflammatory lesions, inflammatory lesions, or a mixture of both, affecting mostly the face but also the back and chest.

Acne vulgaris affects nearly 10% of people worldwide. One of the pathogenic factors for the development of acne is the excessive proliferation of Cutibacterium acnes (C. acnes, also called Propionibacterium acnes or P. acnes), which is the most abundant commensal of human skin and inhabits the pilosebaceous unit in the hair follicle.

Treatment include topical agents, hormonal or oral treatment. For severe acne, a systemic drug, Isotretinoin, also known as 13-cis retinoic acid (13-cis RA), is the only effective treatment. However, this drug is associated with serious side effects including depression, psychosis, anaemia and teratogenicity. Approximately 20% of acne patients develop severe acne and the great amount and severity of side effects resulting from isotretinoin treatment calls for a replacement which still can provide release of acne symptoms.

It has been shown that patients treated with Isotretinoin have a 7-fold increase in Neutrophil gelatinase-associated lipocalin 2 (NGAL) protein, and that this increase results in a decrease of sebum production, which eventually leads to a reduced count of C. acnes. Sebocytes treated with C. acnes have increased NGAL expression and secretion through a TLR-2 dependent mechanism. NGAL has thus been shown to mediate the apoptotic response of Isotretinoin in human sebocytes. Further, NGAL is not only an apoptotic agent but also has antibacterial functions, as it is able to bind to bacterial siderophores and prevents bacteria from receiving the iron necessary for cellular processes. In view of this, the most widely accepted hypothesis of the efficacy observed upon Isotretinoin treatment is that this drug causes an increase in NGAL protein which leads to both sebum reduction and antibacterial activity, reducing the proliferation of P. acnes in the skin of affected people.

In order to use NGAL as an alternative to Isotretinoin in the treatment of acne, the production of the recombinant protein is needed. Proteins can be produced in eukaryotic or prokaryotic cells. However, production systems based on eukaryotic cells are more expensive and less industrially scalable, while prokaryotic protein synthesis can be fairly faster and cheaper because bacteria cells are easier to produce, grow and maintain. Importantly, when a condition affects millions of people worldwide, such as acne or acne-related diseases, the scalability and cost-effectiveness of a treatment represent important factors to be considered.

Some reports show that human recombinant NGAL (rhNGAL) produced in eukaryotic cells can induce apoptosis in SEB-1 sebocytes (Nelson et al. Neutrophil gelatinase-associated lipocalin mediates 13-cis retinoic acid-induced apoptosis of human sebaceous gland cells. J Clin Invest. 2008 April; 118(4):1468-78. doi: 10.1172/JCI33869). At the same time, other reports have shown that recombinant NGAL from prokaryotic origin (produced in E. coli) does not induce apoptosis in TSS-1 sebocytes (Kimberly Ruth Lumsden. The innate immune protein neutrophil gelatinase-associated lipocalin is involved in the early therapeutic response to 13-cis retinoic acid in acne patients. Doctor of Philosophy, May 2011), indicating that posttranslational modifications of NGAL intrinsic to the methods of production (such as glycosylation pattern) may have an impact in the functionality of the protein. Further, due to the antibacterial activity that NGAL has shown in prokaryotic cells, such as E. coli, P. acnes or Mycobacterium tuberculosis (Goetz, Holmes, Borregaard et al. 2002; Martineau, Newton, Wilkinson et al. 2007), strategies aimed at producing this protein using prokaryotic cells have been found not to be possible. Overall, the state of the art indicates that recombinant NGAL produced in bacteria does not have the same function or expression pattern as endogenous NGAL generated in eukaryotic cells, and thus recombinant NGAL produced in eucaryote cells, despite of being more expensive and less scalable, represents the most promising NGAL-based treatment for acne.

The present invention aims at providing an industrially scalable and cost-effective system of producing NGAL protein that is based on the use of prokaryotic cells without hampering the effectiveness of the NGAL protein in reducing sebum production.

On the other hand, a second aim of the present invention is to provide an efficient method to introduce a nucleic acid into C. acnes, which has a very efficient restriction-modification system (R-M system) that functions as a form of bacterial immune system that is able to protect the host bacterium from foreign DNA invasion. Said R-M system consists of two enzyme components, a DNA methyltransferase (MTase) and a restriction endonuclease (REase). While the MTase methylates a specific site in the DNA sequence, the REase cleaves the DNA in a sequence specific manner. Because of the presence of both restriction and modification components of the R-M system, C. acnes is inherently capable of discriminating between the “self” and the “non-self” genetic material. However, this R-M methylation system represents a hurdle to efficiently produce recombinant C. acne, as it efficiently degrades any foreign nucleic acid introduced in this bacterium.

The present invention solved the above problem and provides an improved method to overcome the R-M system of C. acnes and to generate recombinant C. acnes.

### BRIEF DESCRIPTION OF THE INVENTION

In one aspect, the invention provides a method for introducing a nucleic acid into C. acnes, the method comprising the steps of:
a) providing an unmethylated nucleic acid with the proviso that if it comprises a C. acnes methylation motif, said methylated motif is methylated, and
b) introducing said nucleic acid from step a) into C. acnes.

Preferably, the C. acnes methylation motif that is methylated in step a) is AGC(m)AGY and the unmethylated C. acnes methylation motif is AGCAGY.

Preferably, the C. acnes of step b) is obtained by i) having been previously cultured in the presence of at least one permeabilizing agent of bacterial cell wall, and/or ii) having been previously frozen and thawed.

Preferably, the nucleic acid sequence of step a) comprises one or more methylated C. acnes methylation motifs in its sequence, wherein said nucleic acid is obtained by introducing a nucleic acid comprising one or more C. acnes methylation motifs in its sequence into a bacterium that is not C. acnes, wherein the bacterium that is not C. acnes is characterized by comprising or expressing a C. acnes methylase and by not comprising or expressing any other methylase, and subsequently collecting said nucleic acid from said bacteria to proceed with step b). Preferably, the C. acnes methylase is a C. acnes IIIB methylase.

Preferably, the nucleic acid sequence of step a) is unmethylated and does not comprise any C. acnes methylation motifs in its sequence, wherein said nucleic acid sequence is obtained by introducing a nucleic acid that does not comprise any C. acnes methylation motifs in its sequence into a bacterium that is not C. acnes, wherein the bacterium that is not C. acnes is characterized by not expressing any other methylase, and subsequently collecting said nucleic acid from said bacterium to proceed with step b).

Preferably, the bacterium that is not C. acnes is a dam− dcm− hsdMS− E. coli strain, most preferably the E. coli strain deposited in the Spanish Collection of Type Cultures with CECT No. 30749.

Preferably, the nucleic acid comprises C. acnes integration elements that lead the integration of said target nucleic acid or at least a portion thereof in the genome of C. acnes. Preferably, the nucleic acid encodes for the human neutrophil gelatinase-associated lipocalin (NGAL) gene and wherein the recombinant C. acnes is characterized in that it expresses, and preferably secretes, NGAL protein.

Preferably, the C. acnes that have been transformed are selected, and wherein the selection is based on at least two bacterial selection markers.

In another aspect, the invention relates to a recombinant c. acnes obtained by the method of the previous aspect. Preferably, said recombinant C. acnes expresses NGAL protein.

In another aspect, the invention relates to the uses in therapy, diagnosis and in cosmetics of the recombinant C. acnes of the previous aspect. Preferably, the use comprises the topic administration of the recombinant C. acnes into the skin.

In another aspect, the present invention refers to a recombinant Cutibacterium acnes characterized in that it expresses the human neutrophil gelatinase-associated lipocalin (NGAL) protein. Preferably, the NGAL protein has at least 85% amino acid sequence identity over the full length to SEQ ID NO: 1. Preferably, said NGAL is operably linked to a promoter selected from the list consisting of camp2 promoter, camp1 promoter or roxP promoter. In one preferred aspect, the C. acnes expressing NGAL is used in therapy or as a medicament or in medicine, preferably for the treatment of acne vulgaris, urticaria, eczema, rosacea, Hidradenitis suppurativa and/or psoriasis. Another possible use is in cosmetics, preferably to prevent, reduce and/or ameliorate skin aging. Preferably, the use comprises the topic administration of the recombinant C. acnes into the skin.

In a further aspect, the present invention relates to a method for producing the active ingredient of a pharmaceutical composition, wherein said active ingredient is a human neutrophil gelatinase-associated lipocalin (NGAL) protein, wherein the method comprises culturing the recombinant Cutibacterium acnes of the previous aspect.

### DESCRIPTION OF THE FIGURES

FIG. 1. Cytotoxicity assay of rhNGAL on human SZ95 sebocytes incubated with different concentrations from 0, 50, 100, 500, 1000 ng/mL. After 24 h cell viability was measured by MTT assay. Displayed is the average of 4 independent experiments. Cells without treatment were used as a negative control.

FIG. 2. NGAL toxicity study on C. acnes culture. Data represents experimental duplicates treated with 0, 100, 500 or 1000 ng/mL of rhNGAL protein. The used protein was expressed and purified from E. coli BL21 cells.

FIG. 3. Western blot of cytosolic (pellet) and secreted (supernatant) fraction of C. acnes pBR13, pBR14 and pBR16 compared to 40 ug (pBR13), 10 ug (pBR14) and 25 ug (pBR16) of purified protein.

FIG. 4. Sebocytes PCi-SEB Cau were treated with arachidonic acid 5 uM (AA5) to induce sebum production and treated with either 10 uM isotretinoin, rhNGAL produced in (E. coli) or NGAL pBR13 (produced in C. acnes) for 48 h. Lipids were stained by using BODIPY dye and visualized by confocal microscopy. Data is presented as normalized BODIPY intensity after treatment with NGAL from C. acnes pBR13 compared to Isotretinoin (10 uM) treatment and recombinant NGAL protein (50 ng/mL). Results are presented as mean standard deviation (SD) of triplicate measurements (p<0.05) versus vehicle control.

FIG. 5. TDK test (see Shao X, et al. A markerless gene deletion and integration system for Thermoanaerobacter ethanolicus. Biotechnol Biofuels. 2016; 9:100. Published 2016 May 4. doi:10.1186/s13068-016-0514-1).

FIG. 6. Western blot of NGAL protein produced in either HEK293, E. coli or C. acnes. HEK293 produced NGAL is glycosylated and has therefore a higher molecular weight than the E. coli produced protein.

FIG. 7. Functional testing of KO strain on selective agar plates containing no antibiotic/inhibitor, 50 μg/mL FUDR (inhibitor) or 10 μg/mL erythromycin.

FIG. 8. Viability of C. acnes incubated with HEK293 produced NGAL.

FIG. 9. Viability of C. acnes incubated with E. coli produced NGAL.

FIG. 10. Viability of PCi-SEB_Cau sebocytes incubated for 24 h with HEK293 produced human NGAL.

FIG. 11. Viability of SZ95 sebocytes incubated for 24 h with HEK293 produced human NGAL.

FIG. 12. TUNEL assay of PCi-SEB_Cau incubated with NGAL produced in HEK293 (human), E. coli (rh) or C. acnes (pBR13,14,16) at concentrations ranging from 0 μg/mL to 1 μg/mL. Percent of TUNEL positive cells were determined by fluorescence microscopy.

FIG. 13. TUNEL assay of SZ95 incubated with NGAL produced in HEK293 (human), E. coli (rh) or C. acnes (pBR13,14,16) at concentrations ranging from 0 μg/mL to 1 μg/mL. Percent of TUNEL positive cells were determined by fluorescence microscopy.

FIG. 14. Fluorescence measurement of competent cells prepared with different electroporation buffers (EPB) and transformed with fluorescently labelled Cy3 oligonucleotide. DNA internalization is measured by fluorescence intensity. EPB-1: 0.5 M sucrose, 1 mM KOAc, pH 5.8, EPB-2: 30% PEB6000, 6.6% sucrose, EPB-3: 272 mM sucrose, 7 mM NaPi, 1 mM MgCl2, EPB-4: 272 mM sucrose, EPB-5: H20.

FIG. 15. Fluorescence measurement of cells after transformation with Cy3 oligonucleotide compared to 1 h recovery.

FIG. 16. Comparison of fresh cells vs. frozen cells in their transformation efficiency measured in transformants obtained.

FIG. 17. Transformants obtained with DNA mimicking the C. acnes methylation pattern (extracted from E. coli CECT No. 30749) compared to DNA containing the hsdMS methylation pattern (extracted from E. coli dam−dcm− cells).

FIG. 18. Fold change in transformation efficiency comparing various cell-wall-weakening agents in comparison to no pre-treatment. Results represent three independent experiments.

FIG. 19. Fold change in transformation efficiency using MC strains (ZYCY, JMC2, JMC3) with (ermE) or without (ermE RM) AGCAGY motif compared to original pMW535 suicide vector.

FIG. 20. Number of transformants depending on optical density at time of competent cells preparation.

FIG. 21. Number of transformants depending on different DNA amounts transformed.

FIG. 22. Agarose gel of skin samples taken by swabbing followed by DNA extraction and PCR. Junction PCR is shown and compared to control (vehicle) or wild type KPA171202. Only modified strain shows amplification.

FIG. 23. Gram-staining of cryosection of control mice vs. C. acnes colonized mice.

FIG. 24. qPCR of LCN2 gene of skin biopsies compared to control and wild type treated skin (control and wild type samples were taken together in plot).

FIG. 25. SLST and 16S PCR of mice flora at Day 0 and Day 1 after C. acnes application. SLST primers are C. acnes specific and only amplify if C. acnes is present.

FIG. 26. qPCR targeting inflammatory markers TNFα, IL-1β and IL6 of skin biopsies compared to control, wild type or engineered C. acnes treated skin.

FIG. 27. A) Junction PCR of successful replacement of tdk gene by genes coding for LCN2 and ermE. B) Representative graphics of tdk gene replacement by genes encoding NGAL and an erythromycin resistance cassette.

### DESCRIPTION OF THE INVENTION

### General Definitions

It must be noted that, as used herein, the singular forms “a”, “an”, and “the”, include plural references unless the context clearly indicates otherwise. Further, unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the present invention.

The term “about” when referred to a given amount or quantity is meant to include deviations of plus or minus five percent.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. Any of the aforementioned terms (comprising, containing, including, having), whenever used herein in the context of an aspect or embodiment of the present invention may be substituted with the term “consisting of”, though less preferred.

When used herein “consisting of” excludes any element, step, or ingredient not specified in the claim element. When used herein, “consisting essentially of does not exclude materials or steps that do not materially affect the basic and novel characteristics of the claim.

As used herein, the term “NGAL” has its general meaning in the art and refers to the Neutrophil Gelatinase-Associated Lipocalin as described in Schmidt-Ott K M. et al. (2007) (Schmidt-Ott K M, Mori K, Li J Y, Kalandadze A, Cohen D J, Devarajan P, Barasch J. Dual action of neutrophil gelatinase-associated lipocalin. J Am Soc Nephrol. 2007 February; 18(2):407-13. Epub 2007 Jan. 17. Review.). NGAL was shown to exist both as a 25-kDa monomer and a 45-kDa disulfide-linked homodimer, and it may also be covalently complexed with neutrophil gelatinase (also known as matrix metalloproteinase 9, MMP-9) via an intermolecular disulphide bridge as a 135-kDa heterodimeric form.

As used herein, the term “derivative” or “analogue” refers to any substance which is sufficiently structurally similar to the material of which it is identified as a derivative so as to have substantially similar functionality or activity, for example, therapeutic effectiveness, as the material when the substance is used in place of the material.

As used herein, “endogenous gene” or “endogenous promoter” refers to native nucleic acid sequences that are originating from within an organism, that is, that have not external cause. Opposite to “endogenous gene” or “endogenous promoter” is a “exogenous gene” or “exogenous promoter” or “heterologous promoter” or “heterologous gene”, which refer to nucleotide sequences that have an external origin, i.e., that are not native or naturally found in said organism. An example of an exogenous gene, in the context of the present invention, is the nucleic acid encoding for the NGAL protein.

As used herein, the term “retinoid-responsive conditions” refers to a number of pathological conditions that are currently treated using retinoids or to those pathological conditions for which administration of one or more retinoids has a beneficial effect, including those characterized by an excessive production of sebum by the skin, such as acne vulgaris. Other retinoid-responsive conditions rosacea, urticaria, eczema and psoriasis. Retinoids are well-known in the art and include retinol, retinal, tretinoin, isotretinoin, alitretinoin, etretinate, acitretin, tazarotene, bexarotene and adapalene. Preferably, the term “skin’ includes facial or body skin as well as lips.

As used herein, the expression “non-therapeutic use in the cosmetic field” refers to a method used to improve a person's appearance, i.e., to beautify appearance. Preferably, the use in cosmetic includes arresting, reversing, ameliorating, diminishing, and/or reducing defects, imperfections, or aesthetically unpleasant conditions of the skin, which include, but are not limited to: age spots, sunburn, sun spots, lines, fine lines, wrinkles, crow's feet, spider veins, stretch marks, dark eye circles, hyperpigmentation, hypopigmentation, discoloration, uneven skin tone, dullness, freckles, skin breakout, blemishes, skin fragility, dryness, patchiness, tactile roughness, chapping, sagginess, thinning, enlarged pores, cellulite formation, acne formation, rosacea, psoriasis, and eczema. The use in cosmetics does not involve a treatment by surgery or therapy.

The term “isolated” as used herein refers to a substance that has been separated from contaminating cellular components associated with the substance in nature not intended to be associated with the substance and that would interfere with use of the substance in therapeutic, prophylactic, diagnostic or other uses. Generally, an isolated substance described herein is at least about 80% pure, at least about 90% pure, at least about 95% pure, or greater than about 99% pure. Purification is achieved using well-known standard methodology such as fractionation and/or chromatography.

The term “recombinant” refers to protein or bacteria that are not wildtype, that is, that is not found in nature are thus formed by laboratory methods. By “recombinant bacterium” is referred to a bacterium altered, modified or engineered (e.g., genetically engineered) such that it exhibits an altered, modified or different genotype or phenotype (e.g., when the genetic modification affects coding nucleic acid sequences of the microorganism) as compared to the naturally-occurring or starting microorganism from which it was derived.

The terms “treating” and “treatment” used to refer to treatment of a retinoid-responsive condition in a subject includes: preventing, inhibiting or ameliorating the retinoid-responsive condition in a subject, such as slowing progression of the condition and/or reducing or ameliorating a sign or symptom of the condition.

The term “therapeutically effective amount” refers to an amount which produces a desired physiologic or pharmacologic effect in a subject, prevents or ameliorates a condition being treated in the subject. For example, a therapeutically effective amount is an amount which reduces or eliminates a sign or symptom of a retinoid-responsive condition being treated in the subject.

The term “subject” as used herein is a living multi-cellular vertebrate organisms, including, for example, humans, non-human mammals and (non-human) primates.

### Description

As stated above, there is scientific evidence supporting the use of NGAL as an alternative to Isotretinoin in the treatment of acne. However, up to date, the production of NGAL protein can only be based on eukaryotic systems, as NGAL produced in bacteria did not induce apoptosis in the TSS-1 sebocyte model. One possible reason that NGAL produced by bacteria does not function as expected may be because of the specific glycosylation pattern provided by eukaryotic cells. Klausen et al. showed that recombinant NGAL produced in E. coli failed to induce apoptosis in human myeloid bone marrow cells (Klausen, Niemann, Cowland et al. 2005), and NGAL has been reported to be an N-glycosylated protein when expressed in eukaryotic cells. However, prokaryotic cells do not glycosylate proteins in the same manner as mammalian cells, and it may be that the glycosylation of NGAL in eukaryotic cells aides in the recognition of the protein by its receptor.

Bacteria have long been the favourite expression system for recombinant protein production. However, in the case of NGAL production, it is also known that NGAL has antibacterial activity, which means that its production in a bacterial-based system would probably not be workable.

### NGAL Production

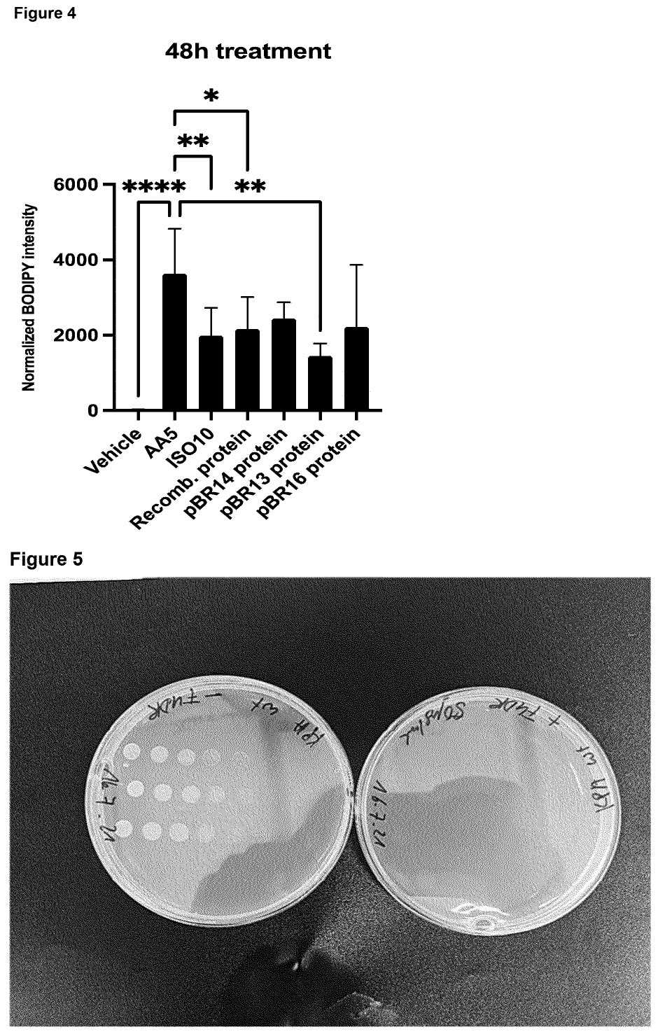

The authors of the present invention have surprisingly found that, although it is reported that NGAL has antibacterial activity against C. acnes, recombinant bacteria (such as P. acnes or E. coli) can indeed be grown and used to produce NGAL. Further, and most importantly, the NGAL produced by C. acnes and E. coli and secreted to the supernatant of the bacterial culture was found effective in reducing sebum in Sebocytes PCi-SEB Cau (see FIG. 4). These results provide evidence that functional NGAL can be recombinantly produced in a bacterial system and that it maintains its inhibitory effect, opening the path for the production of NGAL at an industrially scalable levels and the use of this protein to treat acne worldwide.

In view of this, a first aspect of the present invention relates to a neutrophil gelatinase-associated lipocalin (NGAL) protein derived from the culture of a bacterium, for use in therapy or as a medicament. In an embodiment of the first aspect, the NGAL protein derived from the culture of a bacterium is used in inhibiting sebum production in an epithelial cell. In a preferred embodiment, the neutrophil gelatinase-associated lipocalin (NGAL) protein derived from the culture of a bacterium, is used in the treatment of diseases characterized by an excess of sebum production, preferably acne vulgaris. By “acne vulgaris” is referred herein as the skin condition that occurs when the hair follicles become plugged with sebum and dead skin cells, usually leading the overgrow of bacteria. Acne vulgaris includes the presence of whiteheads, blackheads or pimples, or a combination thereof. More particularly, acne vulgaris include the presence of small red, tender bumps, papules, closed plugged pores, open plugged pores, pustules, nodules, and cystic lesions, or any combination thereof. The acne vulgaris condition may be present in the face, forehead, chest, upper back, and shoulders, or in a combination thereof, of the subject to be treated. The acne vulgaris may be severe or mild acne vulgaris.

Retinoids, a huge family of compounds derived from vitamin A, are known to regulate epithelial cell growth, where they inhibit differentiation of sebaceous glands. As shown in the Examples below, it was found that NGAL produced by recombinant bacteria is able to inhibit sebum production in sebocytes, rendering more than plausible that NGAL according to the first aspect may also be useful in the treatment of other retinoid-responsive diseases, such as urticaria, eczema, dermatitis, rosacea and/or psoriasis.

Preferably, NGAL derived from a culture of a recombinant bacterium is used in inhibiting sebum production in an epithelial cell. Preferably, the epithelial cell is a human skin cell, more preferably a human sebocyte. Sebocytes are highly specialized, sebum-producing epithelial cells that release their content by rupture of the cell membrane and cellular degradation. Sebocytes are terminally differentiated epithelial cells that produce and accumulate lipids (sebum). These cells are most commonly found in the skin in association with hair follicles (forming the pilosebaceous unit), where they arise from hair follicle keratinocytes, but there are also sebaceous glands (SGs) not associated with a hair follicle. Sebum synthesis by sebocytes is strongly regulated by hormones, in particular by androgens. Excessive sebum production is seen in acne vulgaris, one of the most common skin diseases. Histologically, sebocytes can be commonly identified by lipophilic dyes such as Oil Red O, Nile Red and Sudan IV, or by immunostaining against lipid droplet associated proteins such as perilipin and adipophilin, the enzyme fatty acid synthase, keratin 7 and other specific differentiation markers. Methods to measure the sebum inhibiting effect of NGAL provided herein are known in the art and are also illustrated in the examples below. In an embodiment, the NGAL derived from the culture of a recombinant bacterium is capable of cause a reduction in sebum production in a NGAL-treated cell, preferably in a sebocyte, wherein said reduction is in about 1.1, 1.2, 1.3, 1.4, 1.5, 1.6, 1.7, 1.8, 1.9, 2, 2.1, 2.2, 2.3, 2.4, 2.5, 3, 4, 6, 8, 10-fold or more than 10-fold reduction as compared to the sebum production in an untreated cell. In an embodiment, the reduction in sebum production caused by the NGAL provided herein is a statistically significant reduction as compared to sebum production of an untreated cell, preferably an untreated sebocyte.

In a second aspect, the present invention provides non-therapeutic indications or uses of a neutrophil gelatinase-associated lipocalin (NGAL) protein derived from the culture of a bacterium in the cosmetic field. Preferably, the NGAL protein obtained or obtainable from the culture of a bacterium is used with the non-therapeutic purposes of cleaning, beautifying, adding to the attractiveness, altering the appearance, or keeping or promoting the skin or hair in good condition. Said purposes may also be whitening, minimizing the appearance of lines in the face and body, protecting from the sun and sun tanning. In an embodiment, the non-therapeutic use is to prevent, reduce and/or ameliorate skin aging.

In some embodiments, the bacterium according to the first or the second aspect is a skin bacterium that is part of the skin flora or skin microbiota. Skin flora is usually non-pathogenic, and either commensal (are not harmful to their host) or mutualistic (offer a benefit) bacterium that, under normal circumstances, do not cause any skin disease. In an embodiment, the bacterium is a member of the microbiota (communities of microorganisms) that reside on the skin, typically human skin. Preferably, the bacterium is selected from the list consisting of Cutibacterium, Escherichia, Staphylococcus, Corynebacterium, or Micrococcus, or any combination thereof.

In some embodiments, the bacterium according to the first or the second aspect is from the genus Cutibacterium or Escherichia. More preferably, the bacterium is Cutibacterium acnes (C. acnes) or Escherichia coli. Please note that Cutibacterium acnes was formerly known as Propionibacterium acnes (P. acnes), and thus, for the purpose of the present invention, both names are considered synonymous and interchangeable. Cutibacterium acnes is a gram-positive, anaerobic bacteria that normally occupies the hair follicles and sebaceous glands. The Cutibacterium acnes to be used in the present invention can be any, as long as it enables the introduction of a nucleic acid encoding for the NGAL protein and its expression. Preferably, the C. acnes is KPA171202 strain. Escherichia coli, on the other hand, also known as E. coli, is a Gram-negative, facultative anaerobic, rod-shaped, coliform bacterium of the genus Escherichia that is commonly found in the environment, foods, and intestines of people and animals. The E. coli to be used in the present invention to produce the NGAL protein can be any, as long as it enables the introduction of a nucleic acid encoding for the NGAL protein and its expression. Preferably, the E. coli is BL21 strain.

The recombinant bacterium, more preferably C. acnes or E. coli, is characterized in that it comprises a nucleic acid molecule encoding for the NGAL protein. Thus, the recombinant bacterium described herein is thus able to express the NGAL protein. In an embodiment, the nucleic acid molecule encoding for the NGAL protein comprises or consists of a nucleotide sequence with at least 50%, 60%, 70%, 75%, 80%, 85%, 90%, 91%, 92%, 93%, 94%, 95%, 96%, 97%, 98%, 99%, or 100% identity over the full length to SEQ ID NO: 2. Preferably, the NGAL protein expressed by the recombinant bacterium, preferably recombinant C. acnes or E. coli, according to the present invention is encoded by a nucleic acid that consists of SEQ ID NO: 2. In an embodiment, the nucleic acid that encodes for the NGAL protein is codon optimized, preferably codon optimized for the bacterium in which it will be produced, such as codon optimized for E. coli or C. acnes.

In an embodiment, the NGAL protein derived from the culture of a bacterium, for use according to the first or second aspects is from human origin. Preferably, the NGAL protein expressed by the recombinant bacterium, preferably recombinant C. acnes or E. coli, comprises or consists of an amino acid sequence with at least 50%, 60%, 70%, 75%, 80%, 85%, 90%, 91%, 92%, 93%, 94%, 95%, 96%, 97%, 98%, 99%, or 100% identity over the full length with SEQ ID NO: 1. Preferably, the NGAL protein expressed by the recombinant bacterium, preferably recombinant C. acnes or E. coli, consists of SEQ ID NO: 1.

Techniques for determining sequence identity between nucleic acids and amino acids are known in the art. Two or more sequences can be compared by determining their “percent identity.” The percent identity of two sequences, whether nucleic acid or amino acid sequences, is the number of exact matches between two aligned sequences divided by the length of the shorter sequences and multiplied by 100. “Percent (%) amino acid sequence identity” with respect to proteins, polypeptides, antigenic protein fragments, antigens and epitopes described herein is defined as the percentage of amino acid residues in a candidate sequence that are identical with the amino acid residues in the reference sequence (i.e., the protein, polypeptide, antigenic protein fragment, antigen or epitope from which it is derived), after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for example, using publicly available computer software such as BLAST, ALIGN, or Megalign (DNASTAR) software. Those skilled in the art can determine appropriate parameters for measuring alignment, including any algorithms needed to achieve maximum alignment over the full-length of the sequences being compared. The same applies to “percent (%) nucleotide sequence identity”, mutatis mutandis.

Genes encoding NGAL protein are preferably designed to either facilitate secretion of the protein from the bacterium or to facilitate expression of the NGAL protein directly on the surface of the bacterium. In some embodiments, the NGAL protein produced by the bacterium defined herein is released to the extracellular media when the bacterium is growing and thus is metabolic active. Thus, in an embodiment, the NGAL protein derived from the culture of a recombinant bacterium, for use according to the first or the second aspect is obtained from the supernatant media collected from the culture of said recombinant bacterium, preferably the recombinant C. acnes or E. coli. Preferably, the NGAL protein is obtained from the supernatant collected after a period of growth under suitable conditions. Said period may be 6, 8, 10, 12, 14, 16, 18, 20, 22, 24, 48, 72 hours or more. For the protein to be recovered from the supernatant, it should comprise in its amino acid sequence a signal peptide that leads its secretion to the extracellular medium. A signal peptide is a small amino acid sequence whose has function is to direct the protein to the outside of the cytoplasm. Some signal peptides such as PelB, TorA, OmpA represent secretion pathway that is commonly used for extracellular expression in bacteria. Preferably, the signal peptide is placed at the N-terminal of the NGAL protein. In a preferred embodiment, the signal peptide has an amino acid sequence which comprises or consists of SEQ ID NO: 6, 7, or 8, or an amino acid sequence with at least 70%, 75%, 80%, 85%, 90%, 91%, 92%, 93%, 94%, 95%, 96%, 97%, 98%, 99%, or 100% identity over the full length of SEQ ID NO: 6, 7 or 8. In a preferred embodiment, the signal peptide has an amino acid sequence which comprises or consists of SEQ ID NO: 18-33, or an amino acid sequence with at least 70%, 75%, 80%, 85%, 90%, 91%, 92%, 93%, 94%, 95%, 96%, 97%, 98%, 99%, or 100% identity over the full length of SEQ ID NO: 18-33.

Thus, in a preferred embodiment, the NGAL protein obtained or obtainable from the culture of a bacterium, preferably recombinant C. acnes or E. coli, for use according to the first or the second aspects is secreted to the extracellular media, and it comprises or consists of an amino acid sequence with at least 50%, 60%, 70%, 75%, 80%, 85%, 90%, 91%, 92%, 93%, 94%, 95%, 96%, 97%, 98%, 99%, or 100% identity over the full length to SEQ ID NO: 9, 10 or 11. Preferably, the NGAL protein expressed by the recombinant bacterium, preferably recombinant C. acnes or E. coli, consists of SEQ ID NO: 9, 10 or 11.

In addition, for working the present invention, any recombinant NGAL protein such as derivatives or analogues can be used, as long as they are obtained from bacteria. “Derivatives” or “analogues” of NGAL include truncated version of the protein where the function of the NGAL is not altered by said truncation. “Derivatives” or “analogues” also include fusion proteins that are longer than the wild-type NGAL protein due to the addition of extra sequences, such as signal peptides, leader peptides, etc.

In order to obtain a recombinant bacterium that expresses the NGAL protein as defined herein, a nucleotide sequence encoding for said protein should be introduced in a bacterium to provide a recombinant bacterium expressing NGAL. Different means and methods to introduce exogenous nucleotide sequences and thus produce recombinant bacteria are known in the art, such as conventional transformation methods, electroporation, conjugation or protoplast transformation.

The introduction of DNA encoding a heterologous antigen into a bacterium, to produce a recombinant bacterium may be accomplished, for example, by the creation of a recombinant bacterium in which DNA encoding the NGAL protein is harboured on a vector, such as a plasmid. Alternatively, DNA encoding the NGAL protein may be stably integrated into the bacterium chromosome by employing, for example, transposon mutagenesis or by homologous recombination. A preferred method for producing recombinant bacteria comprising a nucleotide encoding for the NGAL protein integrated into the chromosome thereof, is the induction of homologous recombination between a plasmid transfer vector comprising DNA encoding the NGAL protein and bacterium chromosomal DNA. In some embodiments, the nucleotide sequence encoding the NGAL protein, preferably human NGAL protein, is located in a plasmid transfer vector that is transformed into the bacterium, preferably C. acnes or E. coli. Integration of the nucleic acid encoding the human NGAL protein is performed preferentially by homologous recombination. Homologous recombination events are exchanges between DNA molecules (in this case, between bacterial genome and the plasmid transfer vector) that share enough sequence identity to trigger a homologous recombination mechanism between them. Techniques to promote homologous recombination are known in the art, and they lead to the introduction of a desired portion of the plasmid transfer vector into the bacterial genome. To produce by homologous recombination the recombinant bacterium expressing NGAL, preferably the C. acnes or E. coli, a plasmid transfer vector comprising the NGAL-encoding nucleic acid flanked by two regions (one upstream and another downstream of the NGAL coding sequence) of at least 50 base pairs that are homologous to a region in the genome of the bacterium needs to be construed. Once said plasmid transfer vector is provided, it is transformed into the bacterium, and the homologous recombination event between the bacterial genome and the plasmid transfer vector will result in the recombinant bacterium comprising NGAL gene in its genome and thus expressing the NGAL protein. Methods to produce a recombinant bacterium are known in the art, see, e.g., Sörensen et al. Mutagenesis of Propionibacterium acnes and analysis of two CAMP factor knock-out mutants, Journal of Microbiological Methods, Volume 83, Issue 2, 2010, Pages 211-216, ISSN 0167-7012, https://doi.org/10.1016/j.mimet.2010.09.008. Examples of a transfer vector to lead the homologous recombination is provided in the Example section below.

In a preferred embodiment, the nucleic acid encoding the NGAL protein is incorporated into a non-essential region of the genome of the bacterium. Preferably, the nucleic acid sequence encoding for the human NGAL protein is inserted into a gene selected from the list consisting of camp2 (PPA0687), camp1 (PPA1340), roxP (PPA1939), thymidine kinase (tdk) (PPA1049), or Restriction modification IIIB locus (PPA1610, 1611, 1612). Said genes encode for the camp2, camp1, RoxP, Tdk, Restriction modification IIIB proteins, respectively, and thus the insertion of NGAL encoding nucleic acid leads to the interruption or even the replacement of said genes. As shown in FIG. 5, the interruption or replacement of tdk locus represents a promising tool for a knock-out C. acnes expressing NGAL that is able to grow in Brucella plates with FUDR as a negative selection.

For the homologous recombination event to occur specifically in said genes of the bacterial genome, the plasmid transfer vector used should comprise flanking regions that present homology to said genes, wherein said flanking regions are located upstream and downstream of the NGAL-encoding nucleic acid. Thus, in a preferred embodiment, the plasmid transfer vector that will guide the insertion of the nucleic acid sequence encoding the NGAL protein into the bacterial genome comprises a region comprising, in the following order
i) a nucleotide region of at least 50, 100 or 150 base pairs, preferably 200, 250, 300, 350, 400, 452, 500, or more than 500 base pairs, wherein said nucleotide region is at least 70%, 80%, 85%, 90%, 95%, or 100% identical to a nucleotide region of the same length comprised in the bacterial genes camp2 (PPA0687), camp1 (PPA1340), roxP (PPA1939), tdk (PPA1049), or Restriction modification IIIB locus (PPA1610, 1611, 1612),
ii) a NGAL nucleic acid encoding for the human NGAL protein, preferably the NGAL nucleic acid of SEQ ID NO: 2, or a nucleic acid with at least 85%, 90%, 91%, 92%, 93%, 94%, 95%, 96%, 97%, 98%, 99%, or 100% identity over the full length to SEQ ID NO: 2, and
iii) a nucleotide region of least 50, 100 or 150, preferably 200, 250, 300, 350, 400, 452, 500, or more than 500 base pairs, wherein said nucleotide region is at least 70%, 80%, 85%, 90%, 95%, or 100% identical to a nucleotide region of the same length comprised in the bacterial genes camp2 (PPA0687), camp1 (PPA1340) roxP (PPA1939), tdk (PPA1049), or Restriction modification IIIB locus (PPA1610, 1611, 1612,
   so that the NGAL nucleic acid is inserted by homologous recombination into the genome of the bacterium, interrupting the genes camp2 (PPA0687), camp1 (PPA1340) roxP (PPA1939), tdk (PPA1049), or Restriction modification IIIB locus (PPA1610, 1611, 1612) from the bacterial genome, preferably C. acnes or E. coli genomes. An example of said vector for targeting the tdk locus is defined in SEQ ID NO: 15 and in the Examples section below.

Other methods known in the field of gene engineering useful for generating recombinant bacteria can be used, such as, CRISPR, phage integration, or transposon insertion. In one embodiment, the nucleic acid encoding for the NGAL protein is comprised in a plasmid that is transformed into the bacterium. In another embodiment, the nucleic acid construct is a shuttle plasmid. In another embodiment, the nucleic acid construct is an integration vector comprising an integration site. In another embodiment, the nucleic acid construct is a site-specific integration vector. In another embodiment, the nucleic acid construct is any other type of nucleic acid construct known in the art.

It is noted that the nucleotide sequence introduced in the recombinant bacterium may comprise not only the gene encoding for the NGAL protein, but also other elements such as regulatory sequences or selectable markers. In one embodiment, a regulatory sequence is a promoter, while in another embodiment, a regulatory sequence is an enhancer.

In some embodiments, the nucleic acid comprised in the recombinant bacterium comprises an expression cassette comprising the nucleic acid encoding for the NGAL protein, operably linked to a promoter. The promoter driving the expression of NGAL protein may be an endogenous promoter, i.e., a promoter that is naturally found in the bacterium where the NGAL protein is being expressed, preferably C. acnes or E. coli. In this case, it is not necessary to include the promoter in the plasmid transfer vector, as it is already present in the bacterial genome. In some embodiments, the insertion of the nucleic acid encoding for the NGAL protein into the camp2 (PPA0687), camp1 (PPA1340), roxP (PPA1939), tdk (PPA1049), or Restriction modification IIIB locus (PPA1610, 1611, 1612) genes results in said nucleic acid being operably linked to the endogenous camp2, camp1, roxP, Tdk, or Restriction modification IIIB promoters, respectively, so that said endogenous promoters control the expression of the NGAL protein.

In some other embodiments, the promoter is an artificial promoter or an exogenous promoter. In this case, the promoter needs to be supplied by, e.g., the transfer plasmid vector, together with the NGAL encoding gene. In some other embodiments, the camp2, camp1, roxP, Tdk, or Restriction modification IIIB promoters, or any other suitable promoter, is also included in the transfer vector and is flanked by the regions that lead homologous recombination, so that the NGAL nucleotide sequence is not operably linked to an endogenous promoter, but its expression will be driven by the promoter included in the transfer plasmid vector, preferably by camp2, camp1, roxP, Tdk, or Restriction modification IIIB promoters, and which is also inserted into the bacterial genome when the homologous recombination event takes place.

In an embodiment, the NGAL protein obtained or obtainable from the culture of a bacterium, and for use according to the first or second aspects of the present invention, is encoded by a nucleic acid which is operably linked to a promoter selected from the list of camp2, camp1, roxP, Tdk, or Restriction modification IIIB promoters. Preferably the NGAL protein obtained or obtainable from the culture of a bacterium, and for use according to the first or second aspects of the present invention, is encoded by a nucleic acid that is operably linked to a promoter comprising or consisting of a nucleotide sequence with at least 50%, 60%, 70%, 75%, 80%, 85%, 90%, 91%, 92%, 93%, 94%, 95%, 96%, 97%, 98%, 99%, or 100% identity over the full length with SEQ ID NO: 12 (camp1 promoter), 13 (RoxP promoter), or 14 (camp2 promoter).

As described in the examples, several nucleotides were generated comprising NGAL nucleic acid operably linked to one of the promoters camp2, camp1, or roxP, wherein the NGAL proteins produced further comprised a signal peptide (named PPA1939, PPA0687, PPA1340) at their N-terminal (see table 1). Thus, in an embodiment, the NGAL protein obtained or obtainable from the culture of a bacterium, and for use according to the first or second aspects of the present invention, comprises or consists of an amino acid sequence with at least 50%, 60%, 70%, 75%, 80%, 85%, 90%, 91%, 92%, 93%, 94%, 95%, 96%, 97%, 98%, 99%, or 100% identity over the full length to SEQ ID NO: 1, 9, 10, or 11 and said NGAL protein is encoded by a nucleic acid that is operably linked to a promoter comprising or consisting of a nucleotide sequence with at least 50%, 60%, 70%, 75%, 80%, 85%, 90%, 91%, 92%, 93%, 94%, 95%, 96%, 97%, 98%, 99%, or 100% identity over the full length to SEQ ID NO: 12 (camp1 promoter), 13 (RoxP promoter), or 14 (camp2 promoter). Preferably, the NGAL protein obtained or obtainable from the culture of a bacterium, and for use according to the first or second aspects of the present invention, comprises or consists of an amino acid sequence with at least 50%, 60%, 70%, 75%, 80%, 85%, 90%, 91%, 92%, 93%, 94%, 95%, 96%, 97%, 98%, 99%, or 100% identity over the full length to SEQ ID NO: 10 and said NGAL protein is encoded by a nucleic acid that is operably linked to a promoter comprising or consisting a nucleotide sequence with at least 50%, 60%, 70%, 75%, 80%, 85%, 90%, 91%, 92%, 93%, 94%, 95%, 96%, 97%, 98%, 99%, or 100% identity over the full length to SEQ ID NO: 12. Preferably, the NGAL protein obtained or obtainable from the culture of a bacterium, and for use according to the first or second aspects of the present invention, comprises or consists of an amino acid sequence with at least 50%, 60%, 70%, 75%, 80%, 85%, 90%, 91%, 92%, 93%, 94%, 95%, 96%, 97%, 98%, 99%, or 100% identity over the full length to SEQ ID NO: 11 and said NGAL protein is encoded by a nucleic acid that is operably linked to a promoter comprising or consisting a nucleotide sequence with at least 50%, 60%, 70%, 75%, 80%, 85%, 90%, 91%, 92%, 93%, 94%, 95%, 96%, 97%, 98%, 99%, or 100% identity over the full length to SEQ ID NO: 12. Most preferably, the NGAL protein obtained or obtainable from the culture of a bacterium, and for use according to the first or second aspects of the present invention, comprises or consists of an amino acid sequence with at least 50%, 60%, 70%, 75%, 80%, 85%, 90%, 91%, 92%, 93%, 94%, 95%, 96%, 97%, 98%, 99%, or 100% identity over the full length to SEQ ID NO: 9 and said NGAL protein is encoded by a nucleic acid that is operably linked to a promoter comprising or consisting a nucleotide sequence with at least 50%, 60%, 70%, 75%, 80%, 85%, 90%, 91%, 92%, 93%, 94%, 95%, 96%, 97%, 98%, 99%, or 100% identity over the full length to SEQ ID NO: 12.

As stated above, the nucleic acid transformed in the bacterium may comprise the nucleic acid sequence encoding for a selectable marker. The selectable marker may impart resistance to one or more antibiotic agents. For example, selectable markers may include genes for ampicillin resistance, streptomycin resistance, kanamycin resistance, tetracycline resistance, chloramphenicol resistance, sulphonamide resistance, erythromycin resistance, lincomycin resistance or combinations of these markers. Typically, the selectable marker is operationally linked to a promoter that facilitates expression of the marker. Plasmids and other cloning vectors that include selectable markers are known in the art.

Once the step of introducing a nucleic acid encoding for a NGAL protein into a bacterium to obtain a recombinant bacterium expressing NGAL protein, as defined above, is performed, the NGAL protein used according to the first and second aspects is obtained or obtainable from a method comprising the steps of:
a) culturing a recombinant bacterium in a suitable media and under suitable conditions for protein expression, wherein said recombinant bacterium is characterized in that it expresses a human neutrophil gelatinase-associated lipocalin (NGAL) protein, preferably a secretory NGAL protein as defined above,
b) collecting the NGAL protein from the culture of a), preferably from the supernatant of the culture of a),
c) optionally, isolating the NGAL collected in b).

Suitable culture media include any growth medium in a solid, liquid, or semisolid state that allow the growth of the recombinant bacterium and the expression of the NGAL protein. Suitable culture media thus comprise all the elements needed for the bacterium to grow and to be metabolic active, including at least one carbon source, water, salts, and at least one source of amino acid and nitrogen. Several approaches may be employed to express the heterologous antigen in bacteria as will be understood by one skilled in the art once armed with the present disclosure.

The produced NGAL protein may be obtained from the interior of the bacterium, by for instance cell lysis and optionally purification or it may be obtained from the extracellular media if the NGAL protein has been designed to be a secretory protein. In the latter case, the NGAL protein is obtained from the extracellular media directly or after centrifuging said media to discard bacterial cells. Optionally, said NGAL is purified before being used. Protein purification comprises a series of processes intended to isolate one or a few proteins from a complex mixture, such as the culture media where the producer recombinant bacterium is growing. The purification process separates the NGAL protein from all other proteins and compounds present in the media. Separation steps usually exploit differences in protein size, physico-chemical properties, binding affinity and biological activity, which are known by the skilled person. Optionally, the purified protein is further concentrated prior use. Stable transformants of which express NGAL may be isolated and characterized as described herein in the experimental examples.

The NGAL protein derived from the culture of a bacterium, for use according to the first or second aspects of the present invention may be administered to the subject in a single dose, or in multiple (i.e., 2, 3, 4, etc.) doses. A suitable schedule for administration of doses depends on several factors including age, weight, gender, medical history and health status of the subject, type of composition used and route of administration. One of skill in the art is able to readily determine a dose and schedule of administration for a particular subject. Suitable dosages ranges depend on various factors such as the age of the subject, the severity and type of condition being treated in the subject, the general condition of the subject, the route and form of administration of the composition being administered and the particular composition administered. In the case of the therapeutic use according to the first aspect, one of ordinary skill in the art will be able to ascertain a therapeutically effective amount without undue experimentation in view of the art. In some embodiments, the administration may be at the onset of the symptoms of the acne vulgaris condition, for instance, once the first hair follicles become plugged.

In certain embodiments, the subject in need thereof is a human. In certain embodiments, the subject is a teenager, preferably between the ages of 10 and 19 years. In other embodiments, the subject to be treated or in need thereof is an adult. In some embodiments, the subject has acne.

In a particular embodiment of the present invention a method of treating and/or preventing acne in a subject is provided, which includes administering a therapeutically effective amount of NGAL, an NGAL analogue, a supernatant comprising NGAL, or a C. acnes or E. coli expressing NGAL, as defined above.

The subject treated according to the uses of the present invention can be mammalian or non-mammalian. Humans are preferred subjects, although and a mammalian subject can be any mammal including, but not limited to, a non-human primate; a rodent such as a mouse, rat, or guinea pig; a domesticated pet such as a cat or dog; a horse, cow, pig, sheep, goat, or rabbit.

Since the human NGAL protein derived from the recombinant bacterium according to the first or second aspects may be secreted to the extracellular media, in a third aspect, the present invention relates to the supernatant media obtained or obtainable from a culture of a recombinant bacterium, preferably C. acnes or E. coli, wherein the bacterium is characterized by expressing the NGAL protein, wherein said NGAL protein comprises a signal peptide that leads its excretion to the extracellular medium. In some embodiments, the supernatant further comprises bacteria in suspension, preferably recombinant C. acnes or E. coli. In some embodiments, the supernatant has been treated, e.g. by centrifugation or filtration to remove any remaining bacteria. Optionally, the NGAL obtained from the supernatant is purified before being used. The uses defined in the first and second aspect also apply for the supernatant of the third aspect. Thus, the therapeutic use and the non-therapeutic cosmetic use defined above are also uses of the supernatant according to the third aspect.

It is important to note that, although the NGAL protein (purified or comprised in the supernatant) can be used as defined in the above aspects, it is of great interest to also provide a recombinant vector that is able to efficiently produce and deliver said protein preferably in its niche of action, i.e., in the hair follicle, which is surrounded by sebocytes. As shown in Paetzhold et al 2019, C. acnes can engraft and stay on human skin, being a promiser producer and shuttle for the therapeutic NGAL protein directly on human skin. Thus, in a fourth aspect, the present invention relates to a recombinant bacterium, preferably C. acnes or E. coli, characterized in that it comprises a NGAL encoding nucleic acid and characterized in that said recombinant bacterium expresses a neutrophil gelatinase-associated lipocalin (NGAL) protein, preferably a human NGAL. Preferably, the recombinant bacterium, preferably C. acnes or E. coli, is characterized in that it expresses a NGAL protein that comprises or consists of an amino acid sequence with at least 50%, 60%, 70%, 75%, 80%, 85%, 90%, 91%, 92%, 93%, 94%, 95%, 96%, 97%, 98%, 99%, or 100% identity over the full length to SEQ ID NO: 1, 9, 10, or 11. Preferably, the NGAL protein expressed by the recombinant bacterium, preferably recombinant C. acnes or E. coli, consists of SEQ ID NOs: 1, 9, 10, or 11. Preferably, the recombinant bacterium, preferably C. acnes or E. coli, is characterized in that it expresses an NGAL protein that is encoded by a nucleic acid that comprises or consists of a nucleotide sequence with at least 50%, 60%, 70%, 75%, 80%, 85%, 90%, 91%, 92%, 93%, 94%, 95%, 96%, 97%, 98%, 99%, or 100% identity over the full length to SEQ ID NO: 2. Preferably, the recombinant bacterium has been transformed with a nucleic acid molecule that comprises a nucleic acid encoding for the NGAL protein operably linked to a transcription promoter, preferably camp1, camp2 or roxP promoters. Optionally, said NGAL protein is encoded by a nucleic acid sequence that is operably linked and under the control of a promoter comprising or consisting of a nucleotide sequence with at least 50%, 60%, 70%, 75%, 80%, 85%, 90%, 91%, 92%, 93%, 94%, 95%, 96%, 97%, 98%, 99%, or 100% identity over the full length to SEQ ID NO: 12 (camp1 promoter), 13 (RoxP promoter), or 14 (camp2 promoter).

Embodiments regarding the recombinant bacteria, preferably recombinant C. acnes or E. coli, and methods to produce said recombinant bacteria, are explained above and also apply here. The embodiments related to the NGAL protein and its sequence as described in the previous aspects also apply here.

Further, in a fifth aspect, the present invention provides a recombinant bacterium as defined in the fourth aspect, preferably C. acnes or E. coli, characterized in that it expresses a NGAL protein, preferably a human NGAL, said NGAL as defined in any of the previous aspects, for use in therapy or as a medicament. Preferably, the recombinant bacterium is used in inhibiting sebum production in a human skin cell or in the treatment of diseases characterized by an excess of sebum production, such as acne vulgaris. Other uses of the recombinant bacterium according to the fifth aspect include the treatment of other retinoid-responsive diseases, such as urticaria, eczema, dermatitis, rosacea and/or psoriasis. Preferably, the recombinant bacterium for use in therapy or as a medicament is C. acnes, as it provides the benefit of targeted delivery and increased protein penetration since this bacteria is a natural commensal in the hair follicle, being thus in direct contact to human cells like sebocytes.

In a sixth aspect, the present invention provides a recombinant bacterium, preferably C. acnes or E. coli, characterized in that it expresses a NGAL, preferably a human NGAL, as defined in any of the previous aspects, for the non-therapeutic use in the cosmetic field or as a cosmetic composition. In a preferred embodiment, the recombinant bacterium for the non-therapeutic cosmetic use is C. acnes expressing NGAL, preferably human NGAL. Preferably, the recombinant bacterium, preferably C. acnes or E. coli, characterized in that it expresses a NGAL protein, is used with the non-therapeutic purposes of cleaning, beautifying, adding to the attractiveness, altering the appearance, or keeping or promoting the skin or hair in good condition. Said purposes may also be whitening, minimizing the appearance of lines in the face and body, protecting from the sun and sun tanning. In an embodiment, the non-therapeutic cosmetic use is to prevent, reduce and/or ameliorate skin aging. Preferably, the recombinant bacterium is administered alive when used according to the fifth and sixth aspects.

In a seventh aspect, the present invention relates to a composition, preferably a pharmaceutical composition or a cosmetic composition, comprising the human NGAL derived from the culture of the recombinant bacterium as defined above, preferably by C. acnes or E. coli, or a supernatant obtained from the culture of said bacterium, and a pharmaceutically or cosmetically acceptable carriers. Since C. acnes is a common commensal of skin microbiota and can thus be administered, at least topically, to a subject, also included in the seventh aspect of the present invention is a composition, preferably a pharmaceutical or cosmetic composition, comprising the recombinant C. acnes according to any of the previous aspects characterized in that it expresses NGAL protein, wherein the pharmaceutical or cosmetical composition further comprises a pharmaceutically or cosmetically acceptable carrier. Also included in the seventh aspect of the present invention is a composition, preferably a pharmaceutical or cosmetic composition, comprising the recombinant E. coli according to any of the previous aspects characterized in that it expresses NGAL protein, wherein the pharmaceutical or cosmetical composition further comprises a pharmaceutically or cosmetically acceptable carrier.

In some embodiments, the composition comprises a preparation of substantially pure, preferably of up to 80%, 85%, 90%, 95% or 100% pure, isolated NGAL protein derived from the culture of a recombinant bacterium expressing NGAL. In some other embodiments, the composition, preferably a pharmaceutical composition or a cosmetic composition, comprises a preparation of substantially pure, preferably of up to 80%, 85%, 90%, 95% or 100% pure, recombinant C. acnes or E. coli expressing NGAL protein.

Preferably, the pharmaceutical composition comprises a therapeutically effective amount of NGAL protein, preferably isolated NGAL protein, or a therapeutically effective amount of C. acnes, or a combination thereof.

The term “pharmaceutically acceptable carrier” refers to a carrier which is suitable for use in a subject without undue toxicity or irritation to the subject and which is compatible with other ingredients included in a pharmaceutical composition. Pharmaceutically acceptable carriers, methods for making pharmaceutical compositions and various dosage forms, as well as modes of administration are well-known in the art. Other substances that may be comprised in the composition include approved additives, antibiotics, preservatives, adjuvants, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycolic acids, polymeric amino acids, amino acid copolymers, lipid aggregates, or the like.

In some embodiments, the pharmaceutical composition is formulated for topical, local and/or systemic administration to the subject. Formulations to administer the pharmaceutical composition according to the seventh aspect include those suitable for oral, rectal, nasal, pulmonary, ocular, otic, intraarterial, intradermal, intravenous, intramuscular, intraosseous, subcutaneous, topical, transdermal, and transmucosal, such as by sublingual, buccal, vaginal, and inhalational routes of administration.

The pharmaceutical or cosmetic composition according to embodiments of the present invention is in any dosage form suitable for administration to a subject, illustratively including solid, semi-solid and liquid dosage forms such as tablets, capsules, powders, granules, suppositories, pills, solutions, suspensions, ointments, lotions, creams, gels, pastes, sprays and aerosols. For topical administration, a composition can be formulated for administration to the skin such as for local effect, and/or as a “patch” formulation for transdermal delivery. Pharmaceutical formulations suitable for topical administration include, for example, ointments, lotions, creams, gels, pastes, sprays and powders. Ointments, lotions, creams, gels and pastes can include, in addition to one or more active agents, a base such as an absorption base, water-removable base, water-soluble base or oleaginous base and excipients such as a thickening agent, a gelling agent, a colorant, a stabilizer, an emulsifying agent, a suspending agent, a sweetener, a flavouring, or a perfuming agent. Powders and sprays for topical administration of one or more active agents can include excipients such as talc, lactose and one or more silicic acids. Sprays can include a pharmaceutical propellant such as a fluorinated hydrocarbon propellant, carbon dioxide, or a suitable gas. Alternatively, a spray can be delivered from a pump-style spray device which does not require a propellant. A spray device delivers a metered dose of a composition contained therein, for example, using a valve for regulation of a delivered amount. Transdermal formulations can include percutaneous absorption enhancers such as acetone, azone, dimethyl acetamide, dimethyl formamide, dimethyl sulfoxide, ethanol, oleic acid, polyethylene glycol, propylene glycol and sodium lauryl sulfate. Iontophoresis and/or sonophoresis can be used to enhance transdermal delivery.

Liposomes and emulsions are well-known types of pharmaceutical formulations that can be used to deliver a pharmaceutical agent, particularly a hydrophobic pharmaceutical agent Liposomes can include any type of amphipathic materials compatible with a composition to be delivered, illustratively including naturally-occurring lipids, synthetic lipids and combinations thereof.

The term “cosmetically acceptable carrier” refers to a carrier which is suitable for the non-therapeutic use in a subject without undue toxicity or irritation. The cosmetically acceptable carrier may comprise or consist of a cream, gel, serum, balm, sun cream, after sun cream, foundation, tinted cream, tinted sun cream, soothing anti redness cream with green tint, scalp serum, solution, suspension, emulsion, ointment, foam, paste, lotion, powder, soap, surfactant-containing cleansing oil or spray. Dosage forms, as well as modes of administration are well-known in the art. The step of applying the cosmetic composition to the skin may be repeated once or twice daily for up to 8 days. The step of applying the cosmetic composition to the area of skin may be repeated for at least two days, at least three days, at least four days, at least six days, at least seven days, at least 10 days, at least two weeks, at least three weeks, at least four weeks, at least six weeks or at least 8 weeks.

In some embodiments, the composition may be provided in kits for one or more than one administration. In some embodiment, the composition may be applied topically on the skin.

In an eighth aspect, the present invention relates to a method for producing the active ingredient of a pharmaceutical composition, wherein said active ingredient is a NGAL protein that is capable of inhibiting sebum production in an epithelial cell, preferably a sebocyte, wherein the method comprises the steps of:
a) introducing a nucleic acid encoding for a NGAL protein into a bacterium to obtain a recombinant bacteria expressing NGAL protein, preferably a secretory NGAL protein,
b) culturing in a suitable media said recombinant bacterium and collecting the NGAL protein from the culture of a), preferably from the supernatant of the culture of a),
c) optionally, isolating the NGAL collected in b).

In some embodiments, the recombinant bacterium according to any of the aspects above is a skin bacterium that is part of the skin flora or skin microbiota. Skin flora is usually non-pathogenic, and either commensal (are not harmful to their host) or mutualistic (offer a benefit) bacterium that, under normal circumstances, do not cause any skin disease. In an embodiment, the bacterium is a member of the microbiota (communities of microorganisms) that reside on the skin, typically human skin. Preferably, the bacterium is selected from the list consisting of Cutibacterium, preferably C. acnes, Escherichia, preferably E. coli, Staphylococcus, Corynebacterium, or Micrococcus, or any combination thereof.

Since the eighth aspect refers to a NGAL protein and a bacterium producing said protein, all the embodiments disclosed above relating to NGAL protein and recombinant bacteria expressing said NGAL protein are also included in the eighth aspect.

Method to Generate Recombinant C. acnes.

It is known that C. acnes has a highly efficient restriction-modification system (R-M) capable of eliminating any foreign methylated DNA introduced in the bacterium, thereby hampering DNA delivery. This R-M system is the reason why, for C. acnes, an efficient transformation method has not yet been established. Genome manipulation is believed to be a basic tool for functional studies in C. acnes, and therefore there is an urgent need to develop an efficient transformation method that allows the introduction of DNA into this bacterium. The present invention also aims at overcoming this hurdle, and provides, in a ninth aspect, an efficient method to produce recombinant C. acnes.

The authors of the present invention found that a way to increase the transformation efficiency of C. acnes is to provide a nucleic acid capable of completely evading the restriction-modification systems (R-M) of C. acnes. This is possible if the nucleic acid either mimics the methylation pattern of C. acnes so that it is not recognised as foreign nucleic acid, or if the nucleic acid is not methylated at all and does not contain any C. acnes methylation motifs, so that the C. acnes R-M system does not recognise and degrade it. As used herein, “methylation” refers to the process of adding a methyl group to specific regions of the nucleic acid, preferably to specific nucleobases.

Both options were tested in the Examples provided below: First, a Δdam Δdcm ΔhsdMS E. coli strain harboring the C. acnes IIIB methylase was generated. This strain, dubbed EC-24, produces DNA that lacks E. coli-specific methylation but instead mimics the methylation pattern of C. acnes. Thus, shuttling plasmids through EC-24 before delivery into C. acnes should contribute to R-M evasion. While there were very few transformants in the absence of C. acnes-specific methylation, transformation efficiency increased up to 200-fold when plasmid methylation mimicked that of the host bacterium (FIG. 17).

Further, a DNA lacking any methylation was also generated using an E. coli JMC3 strain lacking endogenous R-M systems (Δdam, Δdcm, ΔhsdMS) and that produces minicircles (suicide vectors of reduced size) that are not methylated at all. It was observed that vectors from strains without any R-M system (JMC3 strain) increased the number of transformants over those containing one or more endogenous R-M systems (JMC2 strain: dam+, Δdcm, ΔhsdMS; ZYCY strain: dam+, dcm+, hsdMs+), indicating that DNA delivery into C. acnes is also improved if the DNA does not comprise any methylation pattern or motif that C. acnes may recognize as exogenous.

In view of the above results, the ninth aspect provides a method for introducing a nucleic acid in C. acnes. As used herein, “introducing a nucleic acid” refers to any method to deliver a nucleic acid into a bacterium, such as electroporation, transduction (e.g., injection of a nucleic acid by a bacteriophage), microinjection, by inducing chemical competence (e.g., by addition of alkali cations, cesium, lithium, polyethylene glycol or by osmotic shock), heat shock, conjugation, etc. It is to be understood that “introducing a nucleic acid” is not only the mere introduction but refers to a stable incorporation of the nucleic acid into the bacterium, i.e., that the nucleic acid is not immediately degraded inside the bacterium. In particular, “introducing a nucleic acid in C. acnes” is referred herein to any method to deliver a nucleic acid into a C. acnes, wherein the nucleic acid introduced in C. acnes is not recognised as exogenous from the C. acnes R-M system, preferably by C. acnes REase, and thus it is not degraded upon delivery.

A first embodiment of this aspect comprises a method for introducing a nucleic acid in C. acnes, the method comprising the steps of:
a) providing a nucleic acid sequence suitable for being introduced in C. acnes, and
b) introducing the nucleic acid sequence of a) in C. acnes.
   Step a) Comprises or Consists of Providing a Nucleic Acid Sequence Suitable for being Introduced in C. acnes.

A nucleic acid sequence that is suitable for being introduced in C. acnes is a polynucleotide that is not recognized as exogenous or “non-self” by C. acnes R-M system, and therefore it is not degraded by C. acnes REase(s). In an embodiment, the nucleic acid sequence of step a) is suitable for being introduced in C. acnes when it does not contain a motif which would be recognized by a C. acnes methylase and it is therefore not methylated (i.e., it is an unmethylated nucleic acid sequence) and when, if it comprises one or more C. acnes methylation motifs, they are methylated, being said motifs the only methylated motifs present in the sequence of the nucleic acid. Hence, a nucleic acid sequence that is suitable for being introduced in C. acnes is an unmethylated nucleic acid sequence, with the proviso that if it comprises a C. acnes methylation motif, said methylated motif is methylated. Said nucleic acid will not be degraded upon delivery into C. acnes as it will be recognized as “self” by the C. acnes R-M system. However, if the nucleic acid comprises methylation motifs from other bacterium or organism, or if it comprises unmethylated C. acnes methylation motifs, it will be recognized by the C. acnes R-M system, particularly by the REase, and then it will be degraded. Preferably, the nucleic acid suitable for being introduced in C. acnes either
is not methylated and does not comprise C. acnes methylation motifs, or
it comprises one or more C. acnes methylation motifs that are methylated and does not comprise any other methylated nucleobase other than said methylated motifs.

As used herein, in the context of nucleic acids, a “methylation motif” refers a short nucleotide sequence of about 3-15, preferably 5-10 nucleotides, that is comprised in a longer nucleic acid in the form of a recurring pattern, and which is associated to nucleic acid methylation events. “C. acnes methylation motifs” refer to those methylation motifs of a nucleic acid that are recognized by C. acnes methylase (MTase) and that are methylated by it. The most important methylation pattern in bacteria takes place on adenines, particularly in position N6. Other methylations may occur in the C5 carbon or the N4 amino group of the cytosine residues. C. acnes methylation motifs are known in the art and can be retrieved from open data bases, such as REBASE. In an embodiment, the C. acnes methylation motif is BNNDCNNNNNNGTCCCC (SEQ ID NO: 40), wherein B stands for C or G or T; D stands for A or G or T; and N stands for any A, G, C, T, U.

In a preferred embodiment, the C. acnes methylation motif is AGCAGY (SEQ ID NO: 38), wherein this motif is methylated by C. acnes MTase in the second adenine, in the 5′ to 3′ direction, thereby becoming the methylated C. acnes methylation motif AGC(m)AGY (SEQ ID NO: 39). Thus, preferably, the nucleic acid suitable for being introduced in C. acnes either
is not methylated and does not comprise C. acnes methylation motifs AGCAGY, or
it comprises one or more C. acnes methylation motifs AGCAGY that are methylated in the second adenine (AGC(m)AGY), wherein said nucleic acid sequence does not comprise any other methylated nucleobases other than said C. acnes methylation motifs AGC(m)AGY.

In an embodiment, the nucleic acid sequence provided in step a) comprises one or more methylated C. acnes methylation motifs, wherein at least 50%, 60%, 70%, 75%, 80%, 85%, 90%, 95% or 100% of the C. acnes methylation motifs are methylated, and wherein, preferably, said methylation motifs are AGC(m)AGY. In an embodiment, the nucleic acid sequence provided in step a) comprises less than 10, less than 9, less than 8, less than 7, less than 6, less than 5, less than 4, less than 3, less than 2, preferably less than 1 or 0 unmethylated C. acnes methylation motifs, and wherein preferably, said unmethylated C. acnes methylation motifs are AGCAGY.

As explained above, the nucleic acid sequence of step a) is either not methylated at all and does not comprise C. acnes methylation motifs, or it is not methylated and comprises one or more C. acnes methylation motifs that are methylated. In a preferred embodiment, the nucleic acid sequence of step a) is characterized by comprising one or more C. acnes methylation motifs AGCAGY, wherein said methylation motifs are methylated, wherein the methylation is placed in the second adenine (or the fourth nucleobase in the 5′ to 3′ direction), and wherein the rest of the sequence of the nucleic acid is not methylated. The acid nucleic sequence of step a) thus does not comprise unmethylated AGCAGY motifs, as they are degraded by the REase of C. acnes. Methods to determine whether a nucleic acid sequence comprises methylated C. acnes methylation motifs are known in the art and include Oxford Nanopore Sequencing Technologies, SMRT Sequencing, immunoprecipitation, or bisulfite sequencing (in case of methylated C).

The nucleic acid sequence of step a) can be obtained by introducing it into a bacterium that is not C. acnes, also called herein a shuttle bacterium, and subsequently collecting the nucleic acid sequence from said bacterium to proceed with step b). Methods to collect a nucleic acid from a bacterium or a culture of bacteria, such as a culture of E. coli, are known in the art, and include standard protocols to purify DNA or purify plasmids.

The shuttle bacterium may be a bacterium that has been engineered or modified not to express or comprise any functional methylases, or it may be a bacterium that has been engineered to only comprise or express a functional C. acnes methylase, or a methylase that is capable of reproducing the methylation patterns of the C. acnes methylase. By “functional methylase” is referred herein that the methylase is capable of methylating nucleobases, i.e., is an active methylase. A functional C. acnes methylase is capable of methylating C. acnes methylation motifs, such as AGCAGY.

A nucleic acid sequence lacking methylated nucleobases can be obtained by, for instance, introducing said nucleic acid into a bacterium that is not C. acnes and that is characterized by not comprising or expressing any methylase. Said bacterium not expressing any methylase will not have the ability to methylate the nucleic acid, and thus, when the nucleic acid sequence is collected, it will lack methylated nucleobases or motif. A bacterium that is not C. acnes and that does not comprise or express any methylase can be generated departing from a wild type bacterium that is not C. acnes, such as E. coli, and silencing or inactivating its endogenous methylases or endogenous genes encoding said methylases. If this shuttle bacterium is used, it is important that the nucleic acid sequence is designed so that it does not comprises any C. acnes methylation motifs, such as AGCAGY, since the shuttle bacterium will not be able to methylate them and, if they are not methylated, they will be degraded by C. acnes R-M system, as explained above. The sequence of the nucleic acid can be designed to lack the unmethylated AGCAGY motif by, for instance, substituting the second A by either G or C or T, or by removing or re-coding the whole motif if possible. Once the nucleic acid sequence is collected from this shuttle bacterium, for instance by purifying it after growing the shuttle bacterium for 24 hours, it will lack methylated nucleobases and unmethylated C. acnes methylation motifs, and thus it will be suitable for being introduced in C. acnes, as explained above. Preferably, the shuttle bacterium that is not C. acnes and that has been modified not to express any methylase is E. coli, most preferably dam− dcm− hsdMS− E. coli strain, wherein the E. coli endogenous genes dam, dcm, and hsdMS has been inactivated, or their encoded protein inactivated or silenced.

The nucleic acid of step a) may also be obtained by introducing it into a bacterium that is not C. acnes and that has been modified so that the only methylase that it expresses is a functional C. acnes methylase or a methylase that is capable of reproducing the methylation patterns of C. acnes methylases. This is especially important if the nucleic acid sequence comprises C. acnes methylation motifs that need to be methylated prior introducing said nucleic acid sequence in C. acnes. The shuttle bacterium can be generated departing from a shuttle bacterium whose methylation system has been silenced or inactivated, such as a dam− dcm− hsdMS− E. coli as explained in the paragraph above, and further modifying said bacterium to express or comprise a functional C. acnes methylase. Hence, the shuttle bacterium that is not C. acnes not only comprises a functional C. acnes methylase, but it also does not comprise any other active methylase other than that of C. acnes. By “not comprising any other active methylase other than that of C. acnes” is referred herein that the only methylase that is functional in said bacterium that is not C. acnes is a C. acnes methylase. In other words, it is preferred that the shuttle bacterium used to produce and provide the nucleic acid of step a) is deficient of their endogenous R-M systems but proficient for a C. acnes methylase. Preferably, the shuttle bacterium has silenced or inactivated the genes or proteins of its endogenous adenine methyltransferase (dam), cytosine methyltransferase (dcm), and/or methylase subunit of the EcoKI restriction enzyme (hsdMS), or any combination thereof. If a shuttle bacterium that comprises a functional C. acnes methylase is used, it does not matter that the nucleic acid comprises in its sequence one or more C. acnes methylation motifs, such as AGCAGY, since the shuttle bacteria comprising a functional C. acnes methylase that will methylate them, thereby providing methylated C. acnes methylation motifs, preferably AGC(m)AGY, which are not degraded by C. acnes R-M system. Once the nucleic acid is collected from this shuttle bacterium, for instance by purifying it after growing the shuttle bacterium for 24 hours, it will only be methylated in the C. acnes methylation motif present in its sequence, if any, and thus it will be suitable for being introduced in C. acnes, as explained above.

Preferably, the functional C. acnes methylase expressed by the shuttle bacterium is the C. acnes IIIB methylase (MIIIB). Preferably, the functional C. acnes IIIB methylase protein comprises or consists of an amino acid sequence with at least 50%, 60%, 70%, 75%, 80%, 85%, 90%, 91%, 92%, 93%, 94%, 95%, 96%, 97%, 98%, 99%, or 100% identity over the full length with SEQ ID NO: 17. Preferably, the C. acnes IIIB methylase protein comprised or expressed in the bacterium that is not C. acnes consists of SEQ ID NO: 17.

Preferably, the bacterium used as a shuttle that is not C. acnes is E. coli, preferably a recombinant E. coli that has been modified to express a C. acnes methylase and not to express E. coli endogenous methylases. Preferably, said E. coli is a dam−dcm−hsdMS− E. coli strain, wherein the E. coli endogenous genes dam (DNA cytosine methyltransferase), dcm (DNA cytosine methyltransferase), and hsdMS (methylase subunit of the EcoKI restriction enzyme) has been inactivated, or their encoded protein inactivated or silenced, and wherein said E. coli comprises at least a functional C. acnes methylase, preferably a MIIIB. Preferably, the dam− dcm− hsdMS− E. coli strain expressing C. acnes methylase is the strain deposited by Universitat Pompeu Fabra (Universitat Pompeu Fabra, C. de la Mercé, 12, 08002, Barcelona, Spain) on 11.11.2022, in the Spanish Collection of Type Cultures (Colección Española de Cultivos Tipo, Edificio 3 CUE. Parc Cientific Universitat de Valencia, Catedrático Augustin Escardino 9, 46980 Paterna, Valencia, Spain) under accession number CECT No. 30749, according to the Budapest Treaty on the International Recognition of the Deposit of Microorganisms for the Purposes of Patent. To create CECT No. 30749, a nucleic acid cassette containing the C. acnes methylase plus a gene encoding resistance to kanamycin (KanR) was generated and introduced in a dam− dcm− E. coli strain genome, particularly in the hsdMS locus, thereby replacing the E. coli endogenous R-M system and obtaining a dam− dcm− hsdMS− E. coli strain expressing C. acnes methylase.

As shown in the Examples (FIG. 17 and FIG. 19), the efficiency of the method was improved when a short nucleic acid, i.e., a minicircle, was transformed into C. acnes. Therefore, it is preferable that the nucleic acid of step a) is obtained by introducing it into a shuttle bacterium that is a minicircle production bacterial strain, most preferably a dam− dcm− hsdMS− E. coli strain that produces minicircles. By “minicircle production bacterial strain” is referred herein as a bacterium that expresses a set of inducible minicircle-assembly enzymes, PhiC31 integrase, and I-Scel homing endonuclease, thereby producing minicircles. Minicircles are small circular plasmids that are formed by a site-specific recombination of a parental plasmid causing the removal of sequences responsible for plasmid replication in the production strain. Preferably, the minicircle producer strain is CY10P3S2T (Kay et al. 2010) (System Biosciences), JMC2 and JMC3 (Johnston et al. 2019) E. coli strains.

The nucleic acid sequence of step a) is thus preferably a short nucleic acid, as a minicircle (MC) nucleic acid was delivered into C. acnes with more efficiency than larger nucleic acids. Minicircles are plasmids with a reduced size, and may range between 3.000 base pair or less, 2000 base pairs or less, 1000 base pair or less. Thus, minicircles are plasmid derived from original plasmid that have been reduced to the minimum size. In a preferred embodiment, the nucleic acid has a length of maximum 10000, 8000, 7000, 6000, 5000, or 4000 nucleotides, preferably maximum 3000 nucleotides in length. Preferably the nucleic acid is produced by collecting it from a minicircle production bacterial strain.

Alternatively, the nucleic acid lacking methylated nucleobases of step a) may be obtained by in vitro synthesis of said nucleic acid or PCR or its assembly using classical molecular biology techniques such as restriction digestion and ligation. Both nucleotide synthesis methods and molecular biology techniques are known in the art.

Alternatively, the nucleic acid sequence of step a) may be generated by incubating a non-methylated nucleic acid with a C. acnes methylase. Said C. acnes methylase is preferably MIIIB and can be a purified isolated methylase or it may be comprised in a C. acnes cell lysate.

The nucleic acid sequence of step a) may be any nucleotide sequence or any polynucleotide, preferably DNA. Preferably, the nucleic acid sequence of step a) is a plasmid or is comprised in a vector, such as in a plasmid vector. Preferably, the nucleic acid sequence or polynucleotide is a nucleic acid sequence comprising a gene that encodes for a protein of interest, such as NGAL, so that the recombinant C. acnes obtained from the method of the ninth aspect expresses said protein of interest. In an embodiment, the nucleic acid sequence comprises a replication origin of C. acnes, so that is stays in the cytoplasm of C. acnes as a plasmid. In some embodiments, the nucleic acid is obtained by collecting it from a bacterium that is not C. acnes, wherein said nucleic acid also comprises the replication origin of said bacterium that is not C. acnes.

In an embodiment, the nucleic acid sequence is a nucleic acid capable of integrating into the C. acnes genome, preferably is a suicide vector. In an embodiment, the integration is carried out by CRISPR methodology. In a further embodiment, the integration is carried out by using transposases, phages, recombinases, integrases, or any combination thereof. Preferably, the integration is carried out by homologous recombination between the nucleic acid, or a part thereof, and a non-essential region of the genome of C. acnes. A non-essential region of the genome of C. acnes is a region that is not necessary for the growth and/or viability of a C. acnes. In an embodiment, integration of the nucleic acid sequence or a portion thereof in the genome of C. acnes is performed by homologous recombination, wherein the nucleic acid to be introduced into C. acnes genome is flanked by sequences X and Y, that are identical to sequences X′ and Y′ comprised in the genome of C. acnes.

Methods to design a nucleic acid that is capable of homologous recombination with the genome of a bacterium of interest are known, and they were also explained above. Briefly, for the homologous recombination event to occur specifically in a target region of the bacterial genome, the nucleic acid sequence should comprise flanking regions that present homology to said target region. If the nucleic acid sequence comprises a gene of interest to be introduced in the genome of C. acnes by homologous recombination, said gene should be placed in the nucleic acid between the flanking regions that present homology to the target region of the genome of C. acnes. The length of the flaking regions for homologous recombination has been defined above and applies herein.

The nucleic acid sequence of step a) may comprise a gene that encodes a specific protein, which after expression via transcription and translation fulfils a specific function within C. acnes. A gene sometimes includes segments of DNA involved in producing a polypeptide chain and sometimes includes regions preceding and following a coding region (e.g., an open reading frame) involved in the transcription/translation of a gene product and the regulation of the transcription/translation.

In an embodiment, the nucleic acid sequence of step a) is designed to create a gene knock-out in C. acnes as it is inserted into an endogenous gene of C. acnes. A “gene knock-out” as used herein, refers to a combination of genetic techniques that can render a specific gene inoperable or inactive. In some embodiments a gene knock-out reduces or eliminates expression of a polypeptide from a gene. In certain embodiments the expression of gene is substantially reduced or eliminated. Substantially reduced means that the expression of a gene is reduced by at least 80%, at least 90%, at least 95% or at least 98% when compared to an endogenous level of expression of a gene.

In an embodiment, the nucleic acid sequence of step a) is designed to create a knock-in in C. acnes by inserting into C. acnes an exogenous gene or nucleotide sequence. A “knock-in” as used herein, refers to a combination of genetic techniques that result in the introduction of a nucleic acid, preferably a gene, into C. acnes genome. In some embodiments a gene knock-in produces expression of a polypeptide from the gene integrated into C. acnes genome. In some embodiments the nucleic acid comprises a gene of interest, and a promoter configured to direct the expression of the gene of interest. Preferably, the gene of interest encodes for NGAL, as explained above.

In some embodiments, the nucleic acid introduced in C. acnes is designed to create a C. acnes nutritional auxotrophs. C. acnes nutritional auxotrophs need additional supplements/nutrients in order to grow. It needs these nutrients, like amino acids, because it is unable to synthesize them on its own as a result of a mutation generated by the insertion of the nucleic acid following the method of the ninth aspect. One can control the amount of C. acnes that are in a microenvironment, or one can eliminate a population of C. acnes by depleting the environment of a necessary nutrient. In several embodiments, the C. acnes comprises a genome with a mutation or knock out in a gene that codes for glutamine synthetase. In some embodiments, the C. acnes comprises a genome with a mutation or knock out in a gene that codes for asparagine synthetase, diaminopimelic acid (DAP), alanine racemase, d-alanine aminotransferase, thymidylate synthase, diaminopimelate decarboxylase, thiamine-monophosphate kinase, superoxidase dismutase, pyridoxamine 5′-phosphate oxidase or any combination thereof.

As set out above, the nucleic acid introduced, preferably transformed, in the bacterium may comprise the nucleic acid sequence encoding for a selectable marker. The selectable marker may impart resistance to one or more antibiotic agents. For example, selectable markers may include genes for ampicillin resistance, streptomycin resistance, kanamycin resistance, tetracycline resistance, chloramphenicol resistance, sulphonamide resistance, erythromycin resistance, lincomycin resistance, clindamycin resistance or combinations of these markers. Typically, the selectable marker is operationally linked to a promoter that facilitates expression of the marker. Plasmids and other cloning vectors that include selectable markers are known in the art.

Preferably, the nucleic acid is designed to be inserted into a gene selected from the list consisting of camp2 (PPA0687), camp1 (PPA1340), roxP (PPA1939), thymidine kinase (tdk) (PPA1049), or Restriction modification IIIB locus (PPA1610, 1611, 1612).

Step b) Comprises or Consists of Introducing the Nucleic Acid Sequence of Step a) into C. acnes.

As explained above “introducing the nucleic acid into C. acnes” refers to any method to deliver a nucleic acid into a C. acnes, wherein said nucleic acid is not degraded by C. acnes R-M system as it is not recognised by C. acnes R-M system. A nucleic acid can be introduced into C. acnes in the form of a linear or circular plasmid, for example. Once the nucleic acid sequence of step a) has been introduced in C. acnes, said C. acnes becomes a recombinant C. acnes.

Preferably, the C. acnes is electroporated to introduce the nucleic acid sequence of step a). In the context of the present invention, it is understood that step b) is a stable incorporation of the nucleic acid into the recipient C. acnes, either in its genome, or as a replication-independent or self-replicating plasmid. Hence, step b) could also be defined as introducing the nucleic acid from step a) into C. acnes, wherein the introduction results in the nucleic acid sequence of step a) to be incorporated into C. acnes without being degraded. Methods to know whether a nucleic acid sequence has been introduced in C. acnes without being degraded include growth under selective conditions, colony PCR, sequencing, or the use of a reporter encoded in the nucleic acid that has been introduced, such as a fluorescent or a luminescent reporter.

In an embodiment, the C. acnes is a wild type C. acnes. Preferably, the C. acnes is C. acnes KPA171202 strain or KPA171202 R-M knockout strain lacking a functional IIIB methylase. As it is readily apparent in the context of the present invention, the C. acnes used herein are competent C. acnes, i.e, bacterial cells that has been treated to be able to accept extra-chromosomal DNA or plasmids (naked DNA) from the environment. Methods to prepare competent C. acnes are known by the skilled person.

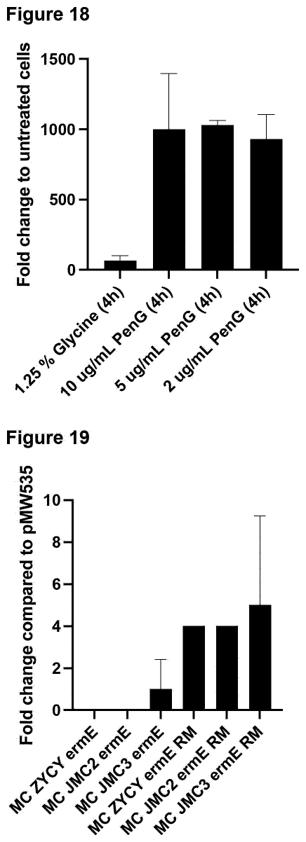

A further optimization developed herein consisted in culturing C. acnes in the presence of at least one cell-wall-permeabilizing agent, such as L-glycine and penicillinG, to facilitate the entry of the nucleic acid into the cell. L-glycine pre-treatment increased efficiency 60-fold, while penicillinG pre-treatment between 2-10 ug/mL increased transformation efficiency up to 1200-fold compared to non-pre-treated controls (FIG. 18). Thus, in an embodiment, the method of the eighth aspect comprises a step of culturing C. acnes previous to the introduction of the nucleic acid in the presence of at least one permeabilizing agent of bacterial cell wall. By “permeabilizing agent of bacterial cell wall” is referred herein as agents that increases the permeability of the outer membrane, preferably by altering the molecular interactions of the lipopolysaccharide constituent of the outer membrane. Thus, the method of the ninth aspect comprises:
a) providing an unmethylated nucleic acid sequence with the proviso that if it comprises a C. acnes methylation motif, said methylated motif is methylated as defined above, and
b) culturing C. acnes in the presence of at least one permeabilizing agent of bacterial cell wall, and subsequently introducing the nucleic acid sequence of step a) into said C. acnes.

In an embodiment, the bacterial cell wall permeabilizing agent is selected from the list consisting of antibiotics (beta lactam and non-beta lactam,), enzymes (mutanolysin, lysozyme, lysostaphin, endolysin, etc.), chemicals (SDS, Triton X-100, Tween-20, EDTA). Most preferably, at least one permeabilizing agent of bacterial cell wall is glycine or penicillin, preferably L-glycine or penicillin G. In an embodiment, the permeabilizing agent of bacterial cell wall is glycine and is used at a concentration range of 0.01%-50%, preferably 1-10%, 1-5%, most preferably 1.25% (v/v %). In an embodiment, the permeabilizing agent of bacterial cell wall is penicillin and is used at a concentration range of 1 ng/mL-1 mg/mL, preferably 1 ng/ml-0.5 mg/ml, 1 μg/ml-10 μg/ml most preferably 5 μg/mL.

In an embodiment, the step of culturing C. acnes in the presence of said permeabilizing agent of bacterial cell wall prior to introducing the nucleic acid sequence is performed by culturing C. acnes for at least 1-48 hours, preferably 1-24 hours or 1-6 hours, more preferably 4 hours.

In an embodiment, the step of culturing C. acnes in the presence of said permeabilizing agent of bacterial cell wall prior to introducing the nucleic acid sequence is performed by culturing C. acnes at a temperature of 37° C., and in a suitable medium for C. acnes culture, in anaerobic conditions and preferably in presence of sucrose for osmotic stabilization. Suitable culture media include any growth medium in a solid, liquid, or semisolid state that allow the growth of C. acnes.

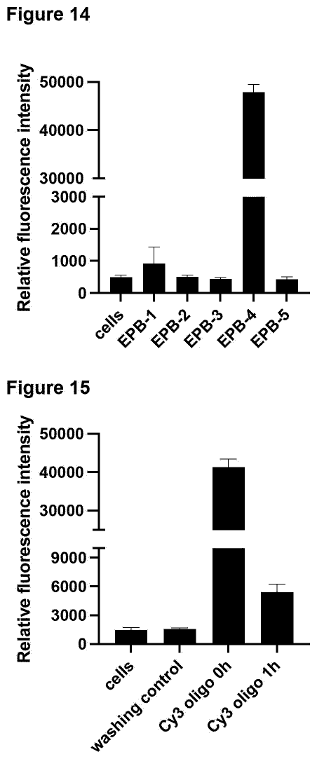

Additionally, as shown in FIG. 14, DNA entry of the fluorescent oligo increased around 50-fold using a buffer containing 272 mM sucrose compared to other electroporation buffers tested (FIG. 14). Thus, in a preferred embodiment, the nucleic acid sequence provided in step a) is introduced in C. acnes by electroporation, wherein the electroporation buffer comprises between 100 μM-1 M of sucrose, preferably between 100-500 mM of sucrose, most preferably 272 mM of sucrose.

Hence, in an embodiment, the method of the ninth aspect comprises:
a) providing an unmethylated nucleic acid sequence with the proviso that if it comprises a C. acnes methylation motif, said methylated motif is methylated, and
b) introducing the nucleic acid sequence of step a) into a C. acnes by electroporation, wherein the electroporation buffer comprises between 100 μM-1 M of sucrose, preferably between 100-500 mM of sucrose, most preferably 272 mM of sucrose.

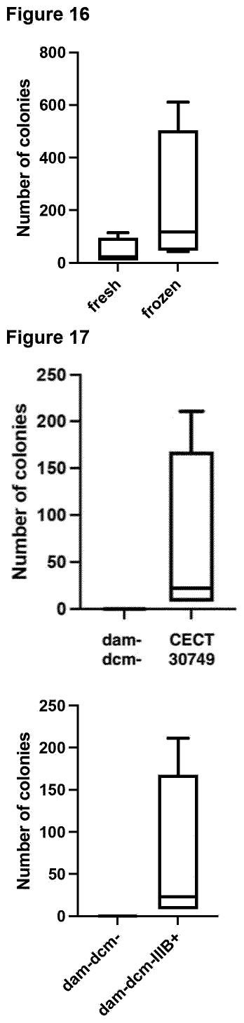

Additionally, transformation efficiency can be also increased by use of a C. acnes that was previously frozen. Freezing the competent cells improved transformation efficiency up to 12-fold over the freshly prepared ones (FIG. 16). Thus, in an embodiment, the method of the eighth aspect comprises freezing C. acnes previous to the introduction of the nucleic acid sequence. Hence, in an embodiment, the method of the ninth aspect comprises:
a) providing an unmethylated nucleic acid sequence with the proviso that if it comprises a C. acnes methylation motif, said methylated motif is methylated, and
b) freezing and thawing C. acnes cells and subsequently introducing said nucleic acid sequence of step a) into said C. acnes, preferably by electroporation.

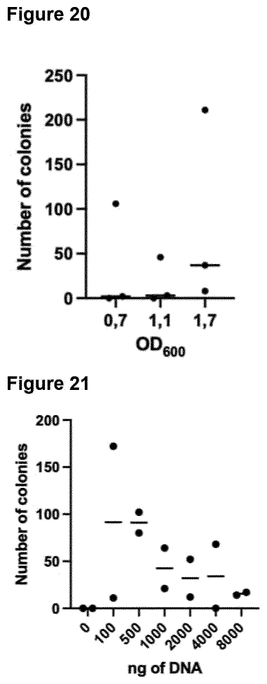

Other factors affecting transformation efficiency were the optical density (OD) of cells at the point of competent cell preparation and the transformed DNA amounts. Lower amounts of DNA and interestingly higher OD were favorable for the outcome (FIGS. 20 and 21). Hence, in a preferred embodiment of the method of the ninth aspect, C. acnes is cultured previous to introduction of the nucleic acid sequence until an optical density (OD) of about 0.1-2.5, preferably 1.7, is reached. Methods to measure OD while culturing the bacteria are known, such as spectrophotometry.

In a further embodiment, C. acnes is cultured previous to introduction of the nucleic acid sequence until an optical density (OD) of about 0.1-2.5, preferably 1.7, is reached, wherein the nucleic acid sequence is prepared at a concentration of between 50 ng-8 ug, preferably 500 ng, to be introduced in C. acnes.

A further step after step b) comprises selecting the recombinant C. acnes in which the nucleic acid sequence has been successfully introduced. Preferably, the recombinant C. acnes are selected by using a suitable selection method, preferably at least one selectable marker. Selection or selectable markers may be positive or negative. Positive selection markers are selectable markers that confer selective advantage to the host organism. An example would be antibiotic resistance, which allows the host organism to survive antibiotic selection. Positive selection markers include 23S rRNA (adenine(2058)-N(6))-methyltransferase (ermE), beta-lactamase (bla), neomycin phosphotransferase (npt) or chloramphenicol acetyltransferase (cat), chloramphenicol resistance gene (cml). Negative or counter selectable markers are selectable markers that eliminate or inhibit growth of the host organism upon selection. An example would be thymidine kinase, which makes the host sensitive to ganciclovir or FUDR (5-Fluoro-2′-deoxyuridine) selection. Negative selection markers include thymidine synthase (thyA), uracil phosphoribosyltransferase (upp), orotidine 5′-phosphate decarboxylase (pyrF), orotate phosphoribosyltransferase (pyrE), uracil phosphoribosyltransferase (pyrR).

In a prefer embodiment, the selection of recombinant C. acnes is performed by using at least two selectable markers, preferably at least one positive and one negative. Combining positive with negative selectable markers is advantageous because it provides a stronger and more efficient selection for the engineered bacterial clones. Preferably, the selection markers are 23S rRNA (adenine(2058)-N(6))-methyltransferase (ermE) and thymidine kinase (tdk).

Preferred embodiments of the eight aspect include:

In an embodiment, the method of the ninth aspect comprises:
a) providing an unmethylated nucleic acid sequence with the proviso that if it comprises a C. acnes methylation motif, said C. acnes methylation motif is methylated, and
b) introducing the nucleic acid sequence of step a) into C. acnes,
   wherein the nucleic acid sequence of a) is obtained by introducing a nucleic acid sequence in a bacterium that is not C. acnes, wherein the bacterium that is not C. acnes comprises or expresses a functional C. acnes methylase and does not comprise or expresses any other functional methylase, and wherein the nucleic acid sequence is collected from said bacterium to provide the nucleic acid sequence of step a), and wherein, preferably, the C. acnes methylation motif is AGCAGY.

In an embodiment, the method of the ninth aspect comprises:
a) providing an unmethylated nucleic acid sequence with the proviso that if it comprises a C. acnes methylation motif, said methylated motif is methylated, and
b) introducing said nucleic acid sequence of step a) into C. acnes,
   wherein the nucleic acid sequence of a) is obtained by introducing a nucleic acid sequence in a bacterium that is not C. acnes, preferably in a dam− dcm− hsdMS− E. coli modified to express a C. acnes methylase, most preferably the bacterium of CECT No. 30749, and wherein the nucleic acid sequence is collected from said bacterium to provide the nucleic acid sequence of step a).

In an embodiment, the method of the ninth aspect comprises:
a) providing a nucleic acid sequence, wherein said nucleic acid sequence:
   i. is not methylated and does not comprise any C. acnes methylation motif, or
   ii. comprises one or more C. acnes methylation motifs, wherein the nucleic acid sequence is methylated only in said C. acnes methylation motifs,
b) introducing said nucleic acid from step a) into C. acnes,
   wherein the nucleic acid sequence of a) is obtained by introducing a nucleic acid sequence in a bacterium that is not C. acnes, preferably in a dam− dcm− hsdMS− E. coli modified to express a C. acnes methylase, most preferably the bacterium of CECT No. 30749, and wherein the nucleic acid sequence is collected from said bacterium to provide the nucleic acid sequence of step a).

In an embodiment, the method of the ninth aspect comprises:
a) providing a nucleic acid sequence comprising one or more C. acnes methylation motifs, wherein said one or more methylated motif are methylated, and wherein the remaining sequence of the nucleic acid sequence is not methylated, and
b) introducing the nucleic acid sequence of step a) in C. acnes, wherein C. acnes has been previously cultured in the presence of at least one permeabilizing agent of bacterial cell wall, preferably L-glycine or penicillin G,
   wherein the nucleic acid sequence of step a) is obtained by introducing a nucleic acid sequence comprising one or more C. acnes methylation motifs into a bacterium that is not C. acnes, wherein said bacterium is characterized by comprising or expressing a functional C. acnes methylase, preferably a C. acnes IIIB methylase, and by not comprising or expressing any other functional methylase, and wherein the nucleic acid sequence is collected from said bacterium to provide the nucleic acid sequence of step a).

In an embodiment, the method of the ninth aspect comprises:
a) providing an unmethylated nucleic acid sequence lacking any C. acnes methylation motif in its sequence, and
b) introducing the nucleic acid sequence of step a) in C. acnes, wherein the C. acnes has been previously cultured in the presence of at least one permeabilizing agent of bacterial cell wall, preferably L-glycine or penicillin G,
   wherein the nucleic acid sequence of a) is obtained by designing a nucleic acid lacking any C. acnes methylation motifs, preferably lacking AGCAGY, in its sequence, and introducing it into a bacterium that is not C. acnes, wherein said bacterium is characterized by not comprising or expressing any functional methylase and thus not being capable of methylating said nucleic acid, and wherein the nucleic acid sequence is collected from said bacterium to provide the nucleic acid sequence of step a).

In an embodiment, the method of the ninth aspect comprises:
a) providing a nucleic acid sequence comprising one or more C. acnes methylation motifs defined by the sequence AGCAGY, wherein said one or more methylated motif are methylated in the second adenine of the sequence (AGC(m)AGY), and wherein the remaining sequence (i.e., the remaining nucleobases) of the nucleic acid sequence is not methylated, and
b) introducing the nucleic acid sequence of step a) in C. acnes, wherein the C. acnes has been previously cultured in the presence of at least one permeabilizing agent of bacterial cell wall, preferably L-glycine or penicillin G,
   wherein the nucleic acid sequence of step a) is obtained by introducing a nucleic acid sequence comprising one or more C. acnes methylation motifs defined by the sequence AGCAGY into a bacterium that is not C. acnes, wherein said bacterium is characterized by comprising or expressing a functional C. acnes methylase, preferably a C. acnes IIIB methylase, and by not comprising or expressing any other functional methylase, and wherein the nucleic acid sequence is collected from said bacterium to provide the nucleic acid sequence of step a).

In a tenth aspect, the present invention provides a recombinant C. acnes obtained or obtainable from the method of the ninth aspect or any of its embodiments. Said C. acnes is characterized by comprising the nucleic acid sequence defined in step a) of the ninth aspect or any of its embodiments. Preferably, the nucleic acid encodes for the NGAL, and the recombinant C. acnes is the recombinant C. acnes of the fourth aspect, which expresses NGAL and which has been preferably obtained with the method of the ninth aspect.

A preferred embodiment of the tenth aspect refers to a composition, preferably a cosmetic or pharmaceutical composition, comprising the recombinant C. acnes obtained or obtainable from the method of the ninth aspect or any of its embodiment. Said compositions may further comprise carriers or additives, or auxiliary substances to formulate the compositions or to administrate the composition in the desired way as explained above in the seventh aspect or any of its embodiments. In some embodiments, a composition is formulated, for example, as a topical (e.g., dermal) formulation. In some embodiments, a composition is formulated, for example, for topical administration to a mammal. A topical formulation may include, for example, a formulation such as a gel formulation, a cream formulation, a lotion formulation, a paste formulation, an ointment formulation, an oil formulation, and a foam formulation. The composition further may include, for example, an absorption emollient. Additional examples of a composition can optionally be formulated to be delivered to the mucosum, or by inhalation, respiration, intranasal, oral, buccal, or sublingual. Chelating agents, buffering agents, cosolvents, preservatives, antioxidants and antimicrobial agents may be added. Supplementary compounds (e.g., biocides and biostats such as antibacterial, antiviral and antifungal agents) can also be added. Compositions comprising recombinant C. acnes have been explained in the seventh aspect and thus said embodiments also apply herein. C. acnes is a common commensal of skin microbiota and can thus be administered, at least topically, to a subject. Also included in the tenth aspect of the present invention is a composition, preferably a pharmaceutical or cosmetic composition, comprising the recombinant C. acnes obtained from the method of the ninth aspect or any of its embodiments.

In an eleventh aspect, the present invention relates to the different uses of the recombinant C. acnes, or compositions comprising said C. acnes, as defined in the of the tenth aspect. The uses of said recombinant C. acnes may vary depending on the features of the nucleic acid introduced in said recombinant C. acnes. The recombinant C. acnes carrying nucleic acid may be used for the controlled expression of peptides as they can move on the skin and propagate deep into the pores and hair follicles allowing absorption of secreted biomolecules. All uses comprise the topical application of said recombinant C. acnes, including when the recombinant C. acnes is alive.

In an embodiment, the recombinant C. acnes may be used in therapy or as pharmaceutical composition. Said recombinant C. acnes can be used to secrete biomolecules (e.g., proteins) to treat disorders in a subject in need. Preferably, the recombinant C. acnes obtained or obtainable with the method of the ninth aspect or any of its embodiments is used in the treatment of skin diseases, preferably diseases related to the excess of seborrheic secretion. Preferably, the use is in the treatment of acne vulgaris, urticaria, eczema, rosacea, Hidradenitis suppurativa and/or psoriasis, as explained above. Other uses of the recombinant C. acnes include in diagnostic methods.

The recombinant C. acnes of the tenth aspect may also be used in cosmetics or as a cosmetic composition. Preferably, said use in cosmetics include arresting, reversing, ameliorating, diminishing, and/or reducing defects, imperfections, or aesthetically unpleasant conditions of the skin, which include, but are not limited to: age spots, sunburn, sun spots, lines, fine lines, wrinkles, crow's feet, spider veins, stretch marks, dark eye circles, hyperpigmentation, hypopigmentation, discoloration, uneven skin tone, dullness, freckles, skin breakout, blemishes, skin fragility, dryness, patchiness, tactile roughness, chapping, sagginess, thinning, enlarged pores, cellulite formation, acne formation, rosacea, psoriasis, and eczema.

In a twelfth aspect, the present invention provides kits including the recombinant C. acnes of the tenth aspect or any of its embodiments. The kit may comprise compositions and pharmaceutical formulations thereof, packaged into suitable packaging material. Kits can be used in various in vitro, ex vivo and in vivo methods and uses, for example a treatment method or use as disclosed herein.

A kit typically includes a label or packaging insert including a description of the components or instructions for use in vitro, in vivo, or ex vivo, of the components therein. A kit can contain a collection of such components, e.g., a C. acnes in combination with a bacterium that is not C. acnes, etc. Kits can include labels or inserts. Labels can include identifying information of one or more components therein, dose amounts, clinical pharmacology of the active ingredient(s) including mechanism of action, pharmacokinetics and pharmacodynamics. Labels or inserts can include information identifying manufacturer information, lot numbers, manufacturer location and date.

Labels or inserts can include information on a condition, disorder, disease or symptom for which a kit component may be used. Labels or inserts can include instructions for the clinician or for a subject for using one or more of the kit components in a method, use, treatment protocol or therapeutic regimen. Instructions can include dosage amounts, frequency or duration, and instructions for practicing any of the methods and uses, treatment protocols or therapeutic regimes set forth herein.

In a thirteenth aspect, the present invention also related to the nucleic acid sequence provided in step a), which is an unmethylated nucleic acid with the proviso that if it comprises a C. acnes methylation motif, said methylated motif is methylated, as defined under the eighth aspect. Thus, all embodiments of the eight aspect in connection to the nucleic acid sequence of step a), and especially on how to obtain it, are included in the thirteenth aspect.

The following clauses are also comprised in the present invention:

1. A human neutrophil gelatinase-associated lipocalin (NGAL) protein derived from a culture of a recombinant bacterium, wherein said NGAL protein is obtained or obtainable from a method comprising the steps of:
a) culturing in a suitable media a recombinant bacterium characterized in that it expresses a human neutrophil gelatinase-associated lipocalin (NGAL) protein, preferably a secretory NGAL protein,
b) collecting the NGAL protein from the culture of a), preferably from the supernatant of the culture of a),
c) optionally, isolating the NGAL collected in b), for use in inhibiting sebum production in a human epithelial cell, preferably in a sebocyte.

2. A human neutrophil gelatinase-associated lipocalin (NGAL) protein derived from a culture of a recombinant bacterium, wherein said NGAL protein is obtained or obtainable from a method comprising the steps of:
a) culturing in a suitable media a recombinant bacterium characterized in that it expresses a human neutrophil gelatinase-associated lipocalin (NGAL) protein, preferably a secretory NGAL protein,
b) collecting the NGAL protein from the culture of a), preferably from the supernatant of the culture of a),
c) optionally, isolating the NGAL collected in b), for use in the treatment of acne vulgaris, urticaria, eczema, rosacea and/or psoriasis.

3. A non-therapeutical cosmetic use of a human neutrophil gelatinase-associated lipocalin (NGAL) protein derived from a culture of a recombinant bacterium, wherein said NGAL protein is obtained or obtainable from a method comprising the steps of:
a) culturing in a suitable media a recombinant bacterium characterized in that it expresses a human neutrophil gelatinase-associated lipocalin (NGAL) protein, preferably a secretory NGAL protein,
b) collecting the NGAL protein from the culture of a), preferably from the supernatant of the culture of a),
c) optionally, isolating the NGAL collected in b),
   preferably to prevent, reduce and/or ameliorate skin aging.

4. The use according to any of clauses 1 or 2, or the method according to clause 3, wherein the recombinant bacterium is Cutibacterium acnes or Escherichia coli.

5. The use according to any of clauses 1 or 2, or the method according to clause 3, wherein the human neutrophil gelatinase-associated lipocalin (NGAL) protein has at least 85% amino acid sequence identity over the full length to SEQ ID NO: 1.

6. The use according to any of clauses 1 or 2, or the method according to clause 3, wherein the recombinant bacterium is Cutibacterium acnes and wherein the human neutrophil gelatinase-associated lipocalin (NGAL) protein has at least 85% amino acid sequence identity over the full length to SEQ ID NO: 1.

7. A method for producing the active ingredient of a pharmaceutical composition, wherein said active ingredient is a human neutrophil gelatinase-associated lipocalin (NGAL) protein that is capable of inhibiting sebum production in a human epithelial cell, preferably in a sebocyte, wherein the method comprises the steps of:
a) culturing in a suitable media a recombinant bacterium characterized in that it expresses a human neutrophil gelatinase-associated lipocalin (NGAL) protein, preferably a secretory NGAL protein,
b) collecting the NGAL protein from the culture of a), preferably from the supernatant of the culture of a),
c) optionally, isolating the NGAL collected in b).

8. The method according to clause 7, wherein the recombinant bacterium is Cutibacterium acnes or Escherichia coli.

9. The method according to clauses 7 or 8, wherein the human neutrophil gelatinase-associated lipocalin (NGAL) protein has at least 85% amino acid sequence identity over the full length to SEQ ID NO: 1.

10. The method according to any of clauses 7 to 9, wherein the recombinant bacterium is Cutibacterium acnes and wherein the human neutrophil gelatinase-associated lipocalin (NGAL) protein has at least 85% amino acid sequence identity over the full length to SEQ ID NO: 1.

11. A recombinant Cutibacterium acnes characterized in that it expresses the human neutrophil gelatinase-associated lipocalin (NGAL) protein.

12. The recombinant Cutibacterium acnes according to clause 11, wherein the human neutrophil gelatinase-associated lipocalin (NGAL) protein has at least 85% amino acid sequence identity over the full length to SEQ ID NO: 1.

13. The recombinant Cutibacterium acnes according to any of the clauses 11 or 12, wherein the nucleic acid encoding for the human neutrophil gelatinase-associated lipocalin (NGAL) is operably linked to a promoter selected from the list consisting of camp2 promoter, camp1 promoter or roxP promoter.

SEQUENCE LISTING
NGAL amino acid sequence without signalling peptide (SEQ ID NO: 1):
QDSTSDLIPAPPLSKVPLQQNFQDNQFQGKWYVVGLAGNAILREDKDPQKMYATIYELKED
KSYNVTSVLFRKKKCDYWIRTFVPGCQPGEFTLGNIKSYPGLTSYLVRVVSTNYNQHAMVF
FKKVSQNREYFKITLYGRTKELTSELKENFIRFSKSLGLPENHIVFPVPIDQCIDG*
NGAL nucleotide sequence without signalling peptide (SEQ ID NO: 2):
CAGGACTCCACCTCCGACCTCATCCCGGCCCCGCCGCTCTCCAAGGTCCCGCTCCAGC
AGAACTTCCAGGACAACCAGTTCCAGGGCAAGTGGTACGTCGTCGGCCTCGCCGGCAA
CGCCATCCTCCGCGAGGACAAGGACCCGCAGAAGATGTACGCCACCATCTACGAGCTC
AAGGAGGACAAGTCCTACAACGTCACCTCCGTCCTCTTCCGCAAGAAGAAGTGCGACTA
CTGGATCCGCACCTTCGTCCCGGGCTGCCAGCCGGGCGAGTTCACCCTCGGCAACATC
AAGTCCTACCCGGGCCTCACCTCCTACCTCGTCCGCGTCGTCTCCACCAACTACAACCA
GCACGCCATGGTCTTCTTCAAGAAGGTCTCCCAGAACCGCGAGTACTTCAAGATCACCC
TCTACGGCCGCACCAAGGAGCTCACCTCCGAGCTCAAGGAGAACTTCATCCGCTTCTC
CAAGTCCCTCGGCCTCCCGGAGAACCACATCGTCTTCCCGGTCCCGATCGACCAGTGC
ATCGACGGC*
PPA1939 secretion peptide nucleotide sequence: (roxP) (SEQ ID NO: 3):
atgttcgtccaaatcgctgccagcctggcagccgcatcgtccattgcactcggcataccaggagctgcc
PPA0687 secretion peptide nucleotide sequence: (Camp2) (SEQ ID NO: 4):
atgaagaagacccatcttgtagctcccctccttgtcggcgcaatgctcgtaccagcggcgctgtcagctcccagtgctcatgct
PPA1340 secretion peptide nucleotide sequence: (camp1) (SEQ ID NO: 5):
atgaaggttaagttcttagcagcgccgctggttgttggtgccttgatggcgccggcagctttctctggagcgacagctcatgct
PPA1939 secretion peptide amino acid sequence: (roxP) (SEQ ID NO: 6):
MFVQIAASLAAASSIALGIPGAA
PPA0687 secretion peptide amino acid sequence: (Camp2) (SEQ ID NO: 7):
MKKTHLVAPLLVGAMLVPAALSAPSAHA
PPA1340 secretion peptide amino acid sequence: (camp1) (SEQ ID NO: 8):
MKVKFLAAPLVVGALMAPAAFSGATAHA
PPA0532 secretion peptide amino acid sequence: (SEQ ID NO: 18):
MWPIRTVRAYCAGTVATAVALAAFVTSGAHA
PPA0533 secretion peptide amino acid sequence: (SEQ ID NO: 19):
MIRSLVATLAVGAIVTAGAQMPAQA
PPA0534 secretion peptide amino acid sequence: (SEQ ID NO: 20):
MLRRAVVGAVTAAVTSTTVLSVVPAGA
PPA0598 secretion peptide amino acid sequence: (SEQ ID NO: 21):
MALRRTLTLMLCAITGASLVATAVPSSA
PPA0644 secretion peptide amino acid sequence: (SEQ ID NO: 22):
MYHHSWHSPDARRRGVTRWATTFIAALTAA
PPA0721 secretion peptide amino acid sequence: (SEQ ID NO: 23):
MEHRYGASQVSGSAPRRGRGVAFAAITGAILLGTVASVDPGAQA
PPA1498 secretion peptide amino acid sequence : (SEQ ID NO: 24):
MMPENEDELTRSVDKAARNTLGRRTFLGGVTLVAAAAVTGTTPALAHA
PPA1662 secretion peptide amino acid sequence: (SEQ ID NO: 25):
MNCSFNRRQRSWLAAMVSGMTVITLVPAAVAHA
PPA1715 secretion peptide amino acid sequence: (SEQ ID NO: 26):
MKQRFKSPTTAVLTGVLIAGSALLLPPLPAKA
PPA2097 secretion peptide amino acid sequence: (SEQ ID NO: 27):
MNHRRRARIAGLSVLCMAAGSFVAKPAFA
PPA2105 secretion peptide amino acid sequence: (SEQ ID NO: 28):
MKINARFAVMAASVAVLMAAAPIAQA
PPA2106 secretion peptide amino acid sequence: (SEQ ID NO: 29):
MRRKSALGFVALSLFATGMGVAAATPATA
PPA2142 secretion peptide amino acid sequence: (SEQ ID NO: 30):
MTFTKKLSALAIAGTMAITGASIATVPAVA
PPA2175 secretion peptide amino acid sequence: (SEQ ID NO: 31):
MTNTSTNAGTTLKRTVALAAAASLAVMGTIAEEAHA
PPA2239 secretion peptide amino acid sequence: (SEQ ID NO: 32):
MSKVVASAIAGALSLTSAGGLTMVQA
PPA2164 secretion peptide amino acid sequence (SEQ ID NO: 33):
MRTDLLRLVSLSRWHNSRREHNMRRSTVKRRTVMAAAAGAFAASALPLSRAAA
NGAL amino acid sequence with signal peptide PPA1939 (SEQ ID NO: 9):
MFVQIAASLAAASSIALGIPGAAQDSTSDLIPAPPLSKVPLQQNFQDNQFQGKWYVVGLAG
NAILREDKDPQKMYATIYELKEDKSYNVTSVLFRKKKCDYWIRTFVPGCQPGEFTLGNIKSY
PGLTSYLVRVVSTNYNQHAMVFFKKVSQNREYFKITLYGRTKELTSELKENFIRFSKSLGLPE
NHIVFPVPIDQCIDG*
NGAL amino acid sequence with signal peptide PPA0687 (SEQ ID NO: 10):
MKKTHLVAPLLVGAMLVPAALSAPSAHAQDSTSDLIPAPPLSKVPLQQNFQDNQFQGKWY
VVGLAGNAILREDKDPQKMYATIYELKEDKSYNVTSVLFRKKKCDYWIRTFVPGCQPGEFTL
GNIKSYPGLTSYLVRVVSTNYNQHAMVFFKKVSQNREYFKITLYGRTKELTSELKENFIRFSK
SLGLPENHIVFPVPIDQCIDG*
NGAL amino acid sequence with signal peptide PPA1340 (SEQ ID NO: 11):
MKVKFLAAPLVVGALMAPAAFSGATAHAQDSTSDLIPAPPLSKVPLQQNFQDNQFQGKW
YVVGLAGNAILREDKDPQKMYATIYELKEDKSYNVTSVLFRKKKCDYWIRTFVPGCQPGEFT
LGNIKSYPGLTSYLVRVVSTNYNQHAMVFFKKVSQNREYFKITLYGRTKELTSELKENFIRFS
KSLGLPENHIVFPVPIDQCIDG*
PPA1340 promoter sequence: (Camp1) (SEQ ID NO: 12):
TAGGTTGACTGGAAAGGGGATATGACACCCATCGTCCTGGGTCTGACAGGGGTAGACA
ATATAGCTTAGAAATGTGTATAAGTAAGATGTTTCTCGCAATGAAAGACGATATGTCACG
AGTTTTCTGGTGCTGTTTCCCGACACGGAACCGTTAGTTTGAATCAATTTTGAACTGAGG
ACACCAAGTTGACCCCCTGGTGTGCAACGATGCTTCTTGCTTGCAGTTGCGAGCAATTG
TTCCCGATGAAAGGAACCCACAA
PPA1939 promoter sequence: (RoxP) (SEQ ID NO: 13):
ACACCGACGATCTGCACGGGCGAGTCTAGGGGTTATCGGCTACATGCGCGTCGCCGCA
TCAGGTTCACGAAACCGAAAGGCAGACCCACCCCCTACCGGGCATACCGTTGCCGCCG
ACACTTCGATGACACTATGCAGTGCTACACTTCAACTGAAAATCTATATGATTTTCGTCA
ATTCCCATACGTACGACACATCGGGAGATCGTC
PPA0687 promoter sequence: (Camp2): (SEQ ID NO: 14):
gcaacaagcctagacgggtctatgcaccgtcgtagccatacaccacacgggcccctgtcggaatgacgcacgcctgatcacag
ctccaacaaatgttggcggcgtacacgccgttcaccacaccgacccccatccaaaatatccgatacacttcgcttgcctcgaccg
gggcatccgcaaccaataaacggagaaccttt
pBR13 full plasmid sequence: suicide vector for targeting tdk locus (5139 bp): (SEQ ID
NO: 15):
UPPERCASE BOLD: PPA1340 promoter sequence: (Camp1) (SEQ ID NO: 12)
lowercase bold: roxP secretion peptide (SEQ ID NO: 3)
underlined: LCN2 gene encoding for NGAL (SEQ ID NO: 2)
ttgccgggaagctagagtaagtagttcgccagttaatagtttgcgcaacgttgttgccattgctacaggcatcgtggtgtcacgctcgt
cgtttggtatggcttcattcagctccggttcccaacgatcaaggcgagttacatgatcccccatgttgtgcaaaaaagcggttagctc
cttcggtcctccgatcgttgtcagaagtaagttggccgcagtgttatcactcatggttatggcagcactgcataattctcttactgtc
atgccatccgtaagatgcttttctgtgactggtgagtactcaaccaagtcattctgagaatagtgtatgcggcgaccgagttgctctt
gcccggcgtcaatacgggataataccgcgccacatagcagaactttaaaagtgctcatcattggaaaacgttcttcggggcgaaaact
ctcaaggatcttaccgctgttgagatccagttcgatgtaacccactcgtgcacccaactgatcttcagcatcttttactttcaccagc
gtttctgggtgagcaaaaacaggaaggcaaaatgccgcaaaaaagggaataagggcgacacggaaatgttgaatactcatactctt
cctttttcaatattattgaagcatttatcagggttattgtctcatgagcggatacatatttgaatgtatttagaaaaataaacaaata
ggggttccgcgcacatttccccgaaaagtgccacctgatgcggtgtgaaataccgcacagatgcgtaaggagaaaataccgcatca
ggaaattgtaagcgttaatattttgttaaaattcgcgttaaatttttgttaaatcagctcattttttaaccaataggccgaaatcggc
aaaatcccttataaatcaaaagaatagaccgagatagggttgagtgttgttccagtttggaacaagagtccactattaaagaacgtgg
actccaacgtcaaagggcgaaaaaccgtctatcagggcgatggcccactacgtgaaccatcaccctaatcaagttttttggggtcg
aggtgccgtaaagcactaaatcggaaccctaaagggagcccccgatttagagcttgacggggaaagccggcgaacgtggcg
agaaaggaagggaagaaagcgaaaggagcgggcgctagggcgctggcaagtgtagcggtcacgctgcgcgtaaccacca
cacccgccgcgcttaatgcgccgctacagggcgcgtccattcgccattcaggctgcgcaactgttgggaagggcgatcggtgcg
ggcctcttcgctattacgccagctggcgaaagggggatgtgctgcaaggcgattaagttgggtaacgccagggttttcccagtcac
gacgttgtaaaacgacggccagtgaattgtaatacgactcactatagggcgaattgggcccgacgtcgcatgctcccggccgcc
atggcggccgcgggAATTCGCCCTTccggccgccatggcgaaggatggcagtcggcagcatggagatcgcgcctcat
gtgggttgacgagctcgttggtgctcttctcgatgagctcgatcctggaaccgccctggtcgtcactgctgatcacggcatggttgac
atccccgacgaccaccgcgtcatggtggaggacgagggtggactgctcgctgacgttgccttgttggctggtgagcctcggttccg
ccacatttacactcgtcgtcccgatgaggtcgcgcaacgatggcgacgcgtactcggggatcgtgctcaggttgtcattggcgacg
atgctgttgcggctgacctgtttggaccagttgacgtgtctacccgttcccgtttcggtgacgtactagcgattactcgtggtgattggg
ccatcatgagccgcgctacccccaaggaactcaccctcgtgggcatgcacggatcgctcactccgtgtgaaatgcggatcccatt
gctggcccagatctgtgaaggatgagggGTACCTAGGTTGACTGGAAAGGGGATATGACACCCATC
GTCCTGGGTCTGACAGGGGTAGACAATATAGCTTAGAAATGTGTATAAGTAAGATGTT
TCTCGCAATGAAAGACGATATGTCACGAGTTTTCTGGTGCTGTTTCCCGACACGGAAC
CGTTAGTTTGAATCAATTTTGAACTGAGGACACCAAGTTGACCCCCTGGTGTGCAACG
ATGCTTCTTGCTTGCAGTTGCGAGCAATTGTTCCCGATGAAAGGAACCCACAAATGGC

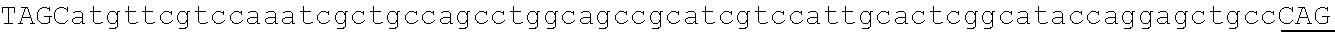

GACTCCACCTCCGACCTCATCCCGGCCCCGCCGCTCTCCAAGGTCCCGCTCCAGCAGA
ACTTCCAGGACAACCAGTTCCAGGGCAAGTGGTACGTCGTCGGCCTCGCCGGCAACGC
CATCCTCCGCGAGGACAAGGACCCGCAGAAGATGTACGCCACCATCTACGAGCTCAAG
GAGGACAAGTCCTACAACGTCACCTCCGTCCTCTTCCGCAAGAAGAAGTGCGACTACTG
GATCCGCACCTTCGTCCCGGGCTGCCAGCCGGGCGAGTTCACCCTCGGCAACATCAAG
TCCTACCCGGGCCTCACCTCCTACCTCGTCCGCGTCGTCTCCACCAACTACAACCAGCA
CGCCATGGTCTTCTTCAAGAAGGTCTCCCAGAACCGCGAGTACTTCAAGATCACCCTCT
ACGGCCGCACCAAGGAGCTCACCTCCGAGCTCAAGGAGAACTTCATCCGCTTCTCCAA
GTCCCTCGGCCTCCCGGAGAACCACATCGTCTTCCCGGTCCCGATCGACCAGTGCATC
GACGGCTCTAGAATCGAAGGTCGTGGCAAGCCGATCCCGAACCCGCTCCTCGGCCTCG
ACTCCACCCGCACCGGCCACCACCATCACCACCATTAGTCGAGCCCGGAGGAGAAGAG
ACTGTGAACACGAACATGGGCCCACCCCTCAAGAGGAATGGGCCCATGCCGTAAATGA
TTTCGATGCAAGTGTCAGGACAACAGCAGGgtaccatggctccaggtcctgacgagcgtggccccagggtt
cgcgtgcggtcaaggattttcccgcggtgggtggtatatcaggtatgcggggggggccgccgaacatgatctcatcccaagaag
ggatcgacgcccgcttcgatttacgcggtttgcgcttcttgcgggggcggctaccctcctcgacgagggcgtcctgctggggagtct
catcggcagcttgagctgacgcttgtgagcgagaagtcgaagtcgaggtcttcttcgtcgatgtgcgtagcttcgacgccgcagtat
cctgcgcggaggtgtcgtcatggggggctgggcctcgcttttccccccagcctgatctggttgggatgtctcgatggcatgcggag
ccccggctggctggggagcctggggcgtagctggctcaggatcctgattcacctccggatgagacgcagcgtcaaggactggg
gtggcgagaccttcatagatgttgaccgagtcctcttgaaacccggcaagcacactagtgaattAAGGGCGaattcgcggcc
gcctgcaggtcgaccatatgggagagctcccaacgcgttggatgcatagcttgagtattctatagtgtcacctaaatagcttggcgt
aatcatggtcatagctgtttcctgtgtgaaattgttatccgctcacaattccacacaacatacgagccggaagcataaagtgtaaag
cctggggtgcctaatgagtgagctaactcacattaattgcgttgcgctcactgcccgctttccagtcgggaaacctgtcgtgccagct
gcattaatgaatcggccaacgcgcggggagaggcggtttgcgtattgggcgctcttccgcttcctcgctcactgactcgctgcgctc
ggtcgttcggctgcggcgagcggtatcagctcactcaaaggcggtaatacggttatccacagaatcaggggataacgcaggaa
agaacatgtgagcaaaaggccagcaaaaggccaggaaccgtaaaaaggccgcgttgctggcgtttttccataggctccgccc
ccctgacgagcatcacaaaaatcgacgctcaagtcagaggtggcgaaacccgacaggactataaagataccaggcgtttccc
cctggaagctccctcgtgcgctctcctgttccgaccctgccgcttaccggatacctgtccgcctttctcccttcgggaagcgtggcgct
ttctcatagctcacgctgtaggtatctcagttcggtgtaggtcgttcgctccaagctgggctgtgtgcacgaaccccccgttcagccc
gaccgctgcgccttatccggtaactatcgtcttgagtccaacccggtaagacacgacttatcgccactggcagcagccactggta
acaggattagcagagcgaggtatgtaggcggtgctacagagttcttgaagtggtggcctaactacggctacactagaagaacag
tatttggtatctgcgctctgctgaagccagttaccttcggaaaaagagttggtagctcttgatccggcaaacaaaccaccgctggta
gcggtggtttttttgtttgcaagcagcagattacgcgcagaaaaaaaggatctcaagaagatcctttgatcttttctacggggtctgac
gctcagtggaacgaaaactcacgttaagggattttggtcatgagattatcaaaaaggatcttcacctagatccttttaaattaaaaat
gaagttttaaatcaatctaaagtatatatgagtaaacttggtctgacagttaccaatgcttaatcagtgaggcacctatctcagcgatc
tgtctatttcgttcatccatagttgcctgactccccgtcgtgtagataactacgatacgggagggcttaccatctggccccagtgctgc
aatgataccgcgagacccacgctcaccggctccagatttatcagcaataaaccagccagccggaagggccgagcgcagaag
tggtcctgcaactttatccgcctccatccagtctattaattg
pBR13 full plasmid sequence: replicative vector derivative of pBRESP36A (9215 bp):
(SEQ ID NO: 16)
UPPERCASE BOLD: PPA1340 promoter sequence: (Camp1) (SEQ ID NO: 12)
lowercase bold: roxP secretion peptide (SEQ ID NO: 3)
underlined: LCN2 gene encoding for NGAL (SEQ ID NO: 2)
GAATTCAAGCTTGTCGACGTTAACCTGCAGGCATGCGGATCCGGTACCAGCCCGACCC
GAGCACGCGCCGGCACGCCTGGTCGATGTCGGACCGGAGTTCGAGGTACGCGGCTTG
CAGGTCCAGGAAGGGGACGTCCATGCGAGTGTCCGTTCGAGTGGCGGCTTGCGCCCG
ATGCTAGTCGCGGTTGATCGGCGATCGCAGGTGCACGCGGTCGATCTTGACGGCTGGC
GAGAGGTGCGGGGAGGATCTGACCGACGCGGTCCACACGTGGCACCGCGATGCTGTT
GTGGGCTGGACAATCGTGCCGGTTGGTAGGATCCAGCGGTGAGCAGTTCGGACGAGC
AGCCGCGCCCGCGTCGCCGCAACCAGGATCGGCAGCACCCCAACCAGAACCGGCCGG
TGCTGGGCCGTACCGAGCGGGACCGCAACCGGCGCCAGTTCGGGCAGAACTTCCTCC
GCGACCGCAAGACCATCGCGCGCATCGCCGAGACAGCCGAGCTGCGGCCCGATCTGC
CGGTGCTGGAAGCCGGCCCCGTCGAAGGGCTGCTCACCAGGGAACTCGCCGACCGCG
CGCGTCAGGTGACGTCGTACGAGATCGACCCCCGGCTGGCGAAGTCGTTGCGGGAGA
AGCTTTCCGGCCACCCGAACATCGAAGTCGTCAACGCCGACTTCCTCACCGCCGAACC
GCCGCCCGAGCCGTTCGCCTTCGTCGGCGCGATCCCCTACGGCATCACCTCGGCGAT
CGTGGACTGGTGCCTGGAGGCGCCGACGATCGAGACGGCGACGATGGTCACGCAGCT
GGAGTTCGCCCGGAAGCGGACCGGCGATTACGGCCGCTGGAGCCGCCTCACGGTGAT
GACCTGGCCGCTGTTCGAGTGGGAGTTCGTCGAGAAGGTCGACCGCCGGCTGTTCAA
GCCGGTGCCCAAGGTCGACTCGGCGATCATGCGGCTGCGCAGGCGCGCCGAACCGCT
GCTGGAAGGCGCGGCGCTCGAACGCTACGAGTCGATGGTCGAGCTGTGCTTCACCGG
CGTCGGCGGCAACATCCAGGCGTCGCTTCTGCGCAAGTACCCGAGGCGCCGCGTCGA
GGCGGCGTTCGACCACGCGGGGGTCGGGGGGGGCGCCGTGGTCGCCTACGTCCGGC
CGGAGCAGTGGCTCCGGCTGTTCGAGCGGCTGGATCAGAAGAACGAACCGAGGGGTG
GGCAGCCCCAGCGGGGCAGGCGAACCGGCGGACGGGACCACGGGGACCGGCGAACC
GGCGGGCAGGATCGCGGCGATCGGCGAACCGGCGGCCGCGACCACAGGGACCGGCA
AGCCAGCGGCCACGGCGATCGTCGCAGCAGCGGACGCAATCGCGACGACGGACGAAC
CGGCGAGCGCGAGCAGGGGGACCAAGGCGGGGGGGGGGGGCCGTCCGGGGGTGGA
CGGACCGGCGGACGTCCAGGGCGACGCGGCGGACCCGGGCAGCGGTAGTCCCCGGC
ACGCGGAACGCGGCAGGCCGTCGAGCGGCCTGCCCCGTTCTGTCGAGAGGAATCAGA
GGTTGATGTCGGCCCGGAGGTCGATGTCGCGCGACGACGAGCCGATCTCCACCGCTC
GCTTGCCGCCCCCGAGCTTCCAGCCGCCCGCGGCTTCGTCCCAGTGCTGGAGGGCCG
CTCCGCGACGTGCACCCGGACGCGCTTGGTCTCGCCCGGTGCGAGTTCGACCTTCTG
GTACCTAGGTTGACTGGAAAGGGGATATGACACCCATCGTCCTGGGTCTGACAGGGG
TAGACAATATAGCTTAGAAATGTGTATAAGTAAGATGTTTCTCGCAATGAAAGACGAT
ATGTCACGAGTTTTCTGGTGCTGTTTCCCGACACGGAACCGTTAGTTTGAATCAATTTT
GAACTGAGGACACCAAGTTGACCCCCTGGTGTGCAACGATGCTTCTTGCTTGCAGTTG
CGAGCAATTGTTCCCGATGAAAGGAACCCACAAATGGCTAGCatgttcgtccaaatcgctgcca
gcctggcagccgcatcgtccattgcactcggcataccaggagctgccCAGGACTCCACCTCCGACCTCAT
CCCGGCCCCGCCGCTCTCCAAGGTCCCGCTCCAGCAGAACTTCCAGGACAACCAGTTC
CAGGGCAAGTGGTACGTCGTCGGCCTCGCCGGCAACGCCATCCTCCGCGAGGACAAG
GACCCGCAGAAGATGTACGCCACCATCTACGAGCTCAAGGAGGACAAGTCCTACAACG
TCACCTCCGTCCTCTTCCGCAAGAAGAAGTGCGACTACTGGATCCGCACCTTCGTCCCG
GGCTGCCAGCCGGGCGAGTTCACCCTCGGCAACATCAAGTCCTACCCGGGCCTCACCT
CCTACCTCGTCCGCGTCGTCTCCACCAACTACAACCAGCACGCCATGGTCTTCTTCAAG
AAGGTCTCCCAGAACCGCGAGTACTTCAAGATCACCCTCTACGGCCGCACCAAGGAGC
TCACCTCCGAGCTCAAGGAGAACTTCATCCGCTTCTCCAAGTCCCTCGGCCTCCCGGA
GAACCACATCGTCTTCCCGGTCCCGATCGACCAGTGCATCGACGGCTCTAGAATCGAA
GGTCGTGGCAAGCCGATCCCGAACCCGCTCCTCGGCCTCGACTCCACCCGCACCGGC
CACCACCATCACCACCATTAGTCGAGCCCGGAGGAGAAGAGACTGTGAACACGAACAT
GGGCCCACCCCTCAAGAGGAATGGGCCCATGCCGTAAATGATTTCGATGCAAGTGTCA
GGACAACAGCAGGATCATCCACGCAATGAAAGCCCGCACCCAATACCTCGACCAGCTC
TAACCAAGAAAGGAACCTGATGAGCGACCAGCTAGACAGCGACCGCAACTACGACCCG
ATGATCTTCGACGTGATGCGCGAGACCGCGAACCGCGTCGTCGCCACGTACGTTGCAT
GGGAAGATGAAGCCGCTGATCCCCGCGAGGCTGCGCACTGGCAGGCCGAGCGATTCC
GCACCCGGCACGAGGTGCGCGCCGTCGACCCTGACAGCCGGCGAGCAGTTCAGGCGA
AGATCGCACAGCTGCGCGAGGAACTAGCCGCAATGCCCGAACACGCCCCAGCCATCC
CTTGGAGCAGGTGGCAGCGTCAGGGGAGTCGGGGGATGTTTGGCAGGGGATGTGGAA
AGAGAGTTCGCTTTGCTCACATGGCTCAACCGGGTAACTAACTGATATGGGGTCTTCGT
CGCCCACTTTGAACACGCCGAGGAATGGACCACGCTGAACGTGACTCGCATGCTTCAC
TGCATGTATGGATTCGTTCGAGACGTTGTTCCCTGAGAGCTGGCTGCCACGCAAGCCG
CTGGCGTCAGCCGAGAAGTCTGGGGCGTACCGGCACGTGACTCGGCAGAGGGCGCTG
GAGCTGCCTTACATCGAAGCGAACCCGTTGGTCATGCAGTCCTTGGTCATCACCGATCG
AGATGCTTCGGATGCTGACTGGGCCGCAGACCTCGCTGGGCTGCCTTCACCGTCCTAC
GTGTCCATGAACCGTGTCACGACCACCGGACACATCGTCTATGCCTTGAAGAACCCTGT
GTGTCTGACCGATGCCGCGCGGCGACGGCCTATCAACCTGCTCGCCCGCGTCGAGCA
GGGCCTATGCGACGTTCTCGGCGGCGATGCATCCTACGGGCACCGGATCACAAAGAAC
CCGCTCAGCACCGCCCATGCGACCCTCTGGGGCCCCGCAGACGCGCTCTACGAGCTG
CGCGCCCTCGCACACACCCTCGACGAGATCCACGCACTGCCGGAGGCAGGGAACCCG
CGTCGCAACGTCACCCGATCAACGGTCGGCCGCAACGTCACCCTGTTCGACACCACCC
GCATGTGGGCATACCGGGCCGTCCGGCACTCCTGGGGCGGCCCGGTCGCCGAATGG
GAGCACACCGTATTCGAGCACATCCACCTACTGAACGAGACGATCATCGCCGACGAATT
CGCCACAGGCCCCCTCGGCTTGAACGAACTTAAGCACTTATCTCGATCCATTTCCCGAT
GGGTCTGGCGCAACTTCACCCCCGAAACCTTCCGCGCACGCCAGAAAGCGATCAGCCT
CCGTGGAGCATCCAAAGGCGGCAAAGAAGGCGGCCACAAAGGCGGCATTGCCAGTGG
CGCATCACGGCGCGCCCATACCCGTCAACAGTTCTTGGAGGGTCTCTCATGACCACAC
GTGAACGTCTCCCCCGCAACGGCTACAGCATCGCCGCTGCTGCGAAAAAGCTCGGTGT
CTCCGAGTCCACCGTCAAGCGGTGGACTTCCGAGCCACGCGAGGAGTTCGTGGCCCG
CGTTGCCGCACGCCACGCGCGGATTCGTGAGCTCCGCTCGGAGGGTCAGAGCATGCG
TGCGATTGCTGCCGAGGTCGGGGTTTCCGTGGGCACCGTGCACTACGCGCTGAACAAG
AATCGAACTGACGCATGACCGTAACGCCGCACGATGAGCATTTTCTTGATCGTGCACCG
CTTGGCACTACGTTCGCGTGCGGTTGCACAGTGCGCGCCACGTTCTTATCCTGCGGCC
ATTGTGGCTACAGCCAATGGGGGGCATCAGCAACGGACGTTGAACCCGGTGGGCAAGT
GTTACTCAGGGGGACATGCCCAGTCTGCGGCGCTCGGATTGACGGTATGGCAGTCGTG
CATGCGGCCCCACCGTCAAACTCATTCAGGTATCAGTGAGAACCCTCATGGCACCCCCT
CGTGACACGTTCTCGTTGCGATCAGCTGCGGCCAAGCTTGATCTTCGGGCAGCGTTGG
GTCCTGGCCACGGGTGCGCATGATCGTGCTCCTGTCGTTGAGGACCCGGCTAGGCTG
GCGGGGTTGCCTTACTGGTTAGCAGAATGAATCACCGATACGCGAGCGAACGTGAAGC
GACTGCTGCTGCAAAACGTCTGCGACCTGAGCAACAACATGAATGGTCTTCGGTTTCCG
TGTTTCGTAAAGTCTGGAAACGCGGAAGTCAGCGCCCTGCACCATTATGTTCCGGATCT
GCATCGCAGGATGCTGCTGGCTACCCTGTGGAACACCTACATCTGTATTAACGAAGCGC
TGGCATTGACCCTGAGTGATTTTTCTCTGGTCCCGCCGCATCCATACCGCCAGTTGTTT
ACCCTCACAACGTTCCAGTAACCGGGCATGTTCATCATCAGTAACCCGTATCGTGAGCA
TCCTCTCTCGTTTCATCGGTATCATTACCCCCATGAACAGAAATCCCCCTTACACGGAG
GCATCAGTGACCAAACAGGAAAAAACCGCCCTTAACATGGCCCGCTTTATCAGAAGCCA
GACATTAACGCTTCTGGAGAAACTCAACGAGCTGGACGCGGATGAACAGGCAGACATC
TGTGAATCGCTTCACGACCACGCTGATGAGCTTTACCGCAGCTGCCTCGCGCGTTTCG
GTGATGACGGTGAAAACCTCTGACACATGCAGCTCCCGGAGACGGTCACAGCTTGTCT
GTAAGCGGATGCCGGGAGCAGACAAGCCCGTCAGGGCGCGTCAGCGGGTGTTGGCG
GGTGTCGGGGCGCAGCCATGACCCAGTCACGTAGCGATAGCGGAGTGTATACTGGCTT
AACTATGCGGCATCAGAGCAGATTGTACTGAGAGTGCACCATATGACCCGAACATGTCG
CGTGGGTGCAAAATGAGCTCAACGAAACACCCCGACAAATCCTCGGCGGCGCAACCCC
ACGTGAAATACTTAACGAACTATTCAAGCGTGGCGCATCCACCGCTTGATTCCGCCTTG
AAGGCATGTTAGAGTTTGGGGCATGTCGAGTCCCGAATCAGACAGGTCAGGGGTTGTC
GGTCACACCGCCAGCCCCTTGAACCACTAGTTACGACGCCCACGATCTGTGTGGGCGT
ATGTCTGGCGTACCCGGGGCGCTGGCCGTGGTGACAAGAAGAACCATTTCTTGATCTC
ACACCTCGGAGTACTCGATGCCTTTTGCCCTCTACATGCTTGCCCTGGCGGTCTTCGTC
ATGGGCACTTCAGAATTCATGCTCGCGGGATTGCTCCCCGCGATCGCGACCGAACTTG
ACGTCTCGGTCGGCACTGCGGGCCTGCTGACCTCCGCATTCGCAGTCGGTATGGTCGT
CGGCGCGCCAGTGATGGCGGCATTCGCTCGCCGTTGGCCACCGCGGCTCACATTGAT
CGTTTGCCTTCTCGTGTTCGCGGGAAGCCACGTCATCGGAGCGATGACACCAGTGTTC
TCTCTCCTGCTCATCACCCGGGTGCTCAGCGCTCTCGCAAACGCAGGATTCCTCGCCG
TAGCACTGAGCACGGCCACTACCCTCGTGCCAGCGAACCAGAAGGGGCGTGCACTGTC
GATCCTGCTCTCCGGCACGACGATCGCAACCGTCGTGGGCGTCCCCGCCGGGGCACT
GCTCGGCACAGCGCTGGGCTGGCGAACGACGTTCTGGGCGATCGCCATCCTCTGTATT
CCCGCGGCCGTTGGAGTCATTCGTGGCGTCACGAACAATGTTGGTCGGAGCGAGACTA
GCGCGACCTCACCAAGGCTCCGTGTCGAGCTCAGCCAGTTGGCGACGCCGCGGCTCA
TCCTGGCCATGGCACTCGGAGCGCTGATCAACGGAGGGACCTTTGCGGCATTCACCTT
CCTGGCACCCATCGTGACCGAGACCGCGGGCTTGGCCGAAGCGTGGGTGTCCGTCGC
GCTGGTGATGTTCGGCATCGGATCGTTCCTTGGCGTCACGATCGCAGGACGACTATCA
GATCAACGACCTGGCCTCGTGCTCGCAGTCGGCGGACCGCTATTGCTGACAGGCTGGA
TCGTGTTGGCAGTGGTCGCATCTCATCCCGTTGCGCTTATCGTCCTCGTCCTCGTTCAG
GGATTCCTGTCGTTCGGCGTCGGCAGTACTCTGATCACGCGTGTGCTGTATGCAGCAT
CGGGTGCGCCAACGATGGGCGGTTCGTACGCAACCGCAGCATTGAATATCGGAGCTGC
AGCGGGGCCCGTGCTTGGTGCGCTCGGGCTCGCGACCGGGCTGGGGCTGCTCGCGC
CGGTTTGGGTCGCTTCGGTGCTGACAGCGATCGCTCTCGTCATCATGCTTCTCACCAGA
CGCGCGCTTACGAAGACCGCGGCGGAGGCCAATTGCATATGCGGTGTGAAATACCGCA
CAGATGCGTAAGGAGAAAATACCGCATCAGGCGCTCTTCCGCTTCCTCGCTCACTGACT
CGCTGCGCTCGGTCGTTCGGCTGCGGCGAGCGGTATCAGCTCACTCAAAGGCGGTAAT
ACGGTTATCCACAGAATCAGGGGATAACGCAGGAAAGAACATGTGAGCAAAAGGCCAG
CAAAAGGCCAGGAACCGTAAAAAGGCCGCGTTGCTGGCGTTTTTCCATAGGCTCCGCC
CCCCTGACGAGCATCACAAAAATCGACGCTCAAGTCAGAGGTGGCGAAACCCGACAGG
ACTATAAAGATACCAGGCGTTTCCCCCTGGAAGCTCCCTCGTGCGCTCTCCTGTTCCGA
CCCTGCCGCTTACCGGATACCTGTCCGCCTTTCTCCCTTCGGGAAGCGTGGCGCTTTCT
CATAGCTCACGCTGTAGGTATCTCAGTTCGGTGTAGGTCGTTCGCTCCAAGCTGGGCTG
TGTGCACGAACCCCCCGTTCAGCCCGACCGCTGCGCCTTATCCGGTAACTATCGTCTT
GAGTCCAACCCGGTAAGACACGACTTATCGCCACTGGCAGCAGCCACTGGTAACAGGA
TTAGCAGAGCGAGGTATGTAGGCGGTGCTACAGAGTTCTTGAAGTGGTGGCCTAACTA
CGGCTACACTAGAAGAACAGTATTTGGTATCTGCGCTCTGCTGAAGCCAGTTACCTTCG
GAAAAAGAGTTGGTAGCTCTTGATCCGGCAAACAAACCACCGCTGGTAGCGGTGGTTTT
TTTGTTTGCAAGCAGCAGATTACGCGCAGAAAAAAAGGATCTCAAGAAGATCCTTTGATC
TTTTCTACGGGGTCTGACGCTCAGTGGAACGAAAACTCACGTTAAGGGATTTTGGTCAT
GAGATTATCAAAAAGGATCTTCACCTAGATCCTTTTAAATTAAAAATGAAGTTTTAAATCA
ATCTAAAGTATATATGAGTAAACTTGGTCTGACAGTTACCAATGCTTAATCAGTGAGGCA
CCTATCTCAGCGATCTGTCTATTTCGTTCATCCATAGTTGCCTGACTCCCCGTCGTGTAG
ATAACTACGATACGGGAGGGCTTACCATCTGGCCCCAGTGCTGCAATGATACCGCGAG
ACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGG
AAGCTAGAGTAAGTAGTTCGCCAGTTAATAGTTTGCGCAACGTTGTTGCCATTGCTACA
GGCATCGTGGTGTCACGCTCGTCGTTTGGTATGGCTTCATTCAGCTCCGGTTCCCAACG
ATCAAGGCGAGTTACATGATCCCCCATGTTGTGCAAAAAAGCGGTTAGCTCCTTCGGTC
CTCCGATCGTTGTCAGAAGTAAGTTGGCCGCAGTGTTATCACTCATGGTTATGGCAGCA
CTGCATAATTCTCTTACTGTCATGCCATCCGTAAGATGCTTTTCTGTGACTGGTGAGTAC
TCAACCAAGTCATTCTGAGAATAGTGTATGCGGCGACCGAGTTGCTCTTGCCCGGCGTC
AATACGGGATAATACCGCGCCACATAGCAGAACTTTAAAAGTGCTCATCATTGGAAAAC
GTTCTTCGGGGCGAAAACTCTCAAGGATCTTACCGCTGTTGAGATCCAGTTCGATGTAA
CCCACTCGTGCACCCAACTGATCTTCAGCATCTTTTACTTTCACCAGCGTTTCTGGGTGA
GCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCAT
GAGCGGATACATATTTGAATGTATTTAGAAAAATAAACAAATAGGGGTTCCGCGCACATT
TCCCCGAAAAGTGCCACCTGACGTCTAAGAAACCATTATTATCATGACATTAACCTATAA
AAATAGGCGTATCACGAGGCCCTTTCGTCTTCAA
C. acnes IIIB methylase protein (SEQ ID NO: 17)
MTAQRLQLTWYNKDKALIPTETGKYGYTWVDPSDPRYCETHTLVLDDYVQGSQTPKSDEF
AYSERADLEPQDDNLLILGESGDVLEALTRVPELAEKYVGKVKLIYIDPPFNTAQTFASYEDN
LEHSIWLTMMRDRLHHMKKLLADDGSIWVHLDYAENHRMRLLLDEVFGCSNFIAEFVWQKA
DSPRGDAQRVSVDQDVILCYAASGSTVMNRMERTAADNARFSNPDGDSKGVWFSDNRSA
PTNVMSWQHPSTFAIQHPISGEMIYPAKGGCWRFGRERLLESLNEYAEYASGDVDIAARVA
NTSLRSDQVRSDIPDLVLVDPASAAQCARTRIDDGNWPEFFVTATSFGRKSYPPNEGQPAR
SWWPNDQVGHNREAKSEIKALFSGATPFSTPKPERLLERIIHIGSNPGDIVLDVFAGSGTTAA
VAQKMGRRWVTCELLESTFTTFTRPRLEKVLNDQDPGGITRTKGERVDATEDGLPDGVSP
EDAAKFTSVLNKLIKDDPELKKSIEVKTLKAASKTRRTKEVVNWRGGGGFQVAHLSPACFDY
APELDRVMLTAAATGQTLIESVTANLGFTLLHPDDDYIFDARRGNALLKVVEGVATTEIVDWL
ASQIQPGETIVLAATTVMDGVRQHLRKLVKGSRVVALPDDVFRYSEGGDQ

### EXAMPLES

### Example 1: Methods

### Bacterial Culture

C. acnes KPA171202 was grown on Brucella agar plates from glycerol and incubated for 3 days, 37° C. anaerobically using GasPak EZ anaerobe pouch system (BD). When grown in liquid, C. acnes KPA171202 was inoculated to an OD₆₀₀ of 0.1 and grown in Brain-Heart Infusion broth (BHI) (Sigma) at 37° C., 110 rpm, anaerobically using AnaeroGen system (Thermo Scientific), until reaching the desired optical density. E. coli DH5a (NZYTech) was used for routinely cloning and E. coli dam−dcm− (New England Biolabs) to create a C. acnes methylase-proficient shuttle strain. E. coli strains were grown in LB medium either supplemented with Ampicillin 50 μg/mL or Kanamycin 50 μg/mL and grown at 37° C. at 225 rpm.

### Cloning of Different Variations of Promoter and Signalling Peptides:

Six different constructs with 2 promoters and 3 secretion peptides were cloned in pGEM-Teasy vector together with 500 bp homology upstream and downstream of gene to be replaced (camp2, camp1, roxP, tdk or others) as previously described in Meike Sörensen, et al. (https://doi.org/10.1016/j.mimet.2010.09.008), Nazipi S, et al. (The Skin Bacterium Propionibacterium acnes Employs Two Variants of Hyaluronate Lyase with Distinct Properties. Microorganisms. 2017 Sep. 12; 5(3):57. Doi: 10.3390/microorganisms5030057). And Allhorn, M et al. (A novel enzyme with antioxidant capacity produced by the ubiquitous skin colonizer Propionibacterium acnes. Sci Rep 6, 36412 (2016). https://doi.org/10.1038/srep36412) or cloned into pBRESP36A plasmid. pBRESP36A plasmid is a replicative plasmid in C. acnes, and the transformation efficiency with this vector (or derivatives) is much higher than with the suicide vector for homologous recombination, making it more suitable for protein production studies (see Jore J P, et al. Efficient transformation system for Propionibacterium freudenreichii based on a novel vector. Appl Environ Microbiol. 2001; 67(2):499-503. doi:10.1128/AEM.67.2.499-503.2001). All gene constructs were fused to a C-terminal histidine tag for purification or western blot analysis.

TABLE 1
construct, promoter name and secretion peptide name.

| Construct | Promoter name | Secretion peptide name |
|---|---|---|
| pBR13 | PPA1340 | PPA1939 |
| pBR14 | PPA1340 | PPA0687 |
| pBR16 | PPA1340 | PPA1340 |
| pBR18 | PPA1939 | PPA1939 |
| pBR19 | PPA1939 | PPA0687 |
| pBR20 | PPA1939 | PPA1340 |

Recombinant NGAL Protein Purification of C. acnes Supernatant

Proteins of 100 mL filtered supernatant were purified by a HiTrap Ni²⁺ column followed by desalting and a size exclusion. Total proteins were quantified by Qubit Protein assay kit (Invitrogen), concentrations and final protein yields of pBR13 (0.46 mg), pBR14 (0.14 mg), pBR16 (0.50 mg) obtained.

C. acnes Culture for Protein Production and Secretion

Transformed C. acnes KPA171202 was inoculated in 50 mL BHI medium supplemented with 10 μg/mL erythromycin to a starting OD600 of 0.1 and grown anaerobically for 48 h until reaching an OD of 1.2. Cells were spun down for 10 min, 5000 rpm. Pellet was resuspended in 1 mL S30 buffer, lysed by a FastPrep FP120 for 25 sec, speed 6.5, 2 cycles and centrifuged full speed, 30 min at 4 degrees. The supernatant was filtered through 0.22 um. Both filtered supernatant and lysate were used for TCA precipitation of secreted proteins.

Deoxycholate-Trichloroacetic Acid (TCA) Precipitation of C. acnes Produced NGAL Protein

Proteins from lysate and 50 mL filtered supernatants were TCA precipitated. In summary, protein containing supernatants were treated for 10 min at RT with deoxycholate (DOC) and then precipitated using 10% trichloroacetic acid (TCA) (Sigma Aldrich) on ice for 30 min. Samples were centrifuged for 10000×g, 4° C., 15 min and the protein pellet was washed twice in pure ice-cold acetone, air-dried and resuspended in 100 μl of 25 mM Tris-HCl buffer (pH 8.0).

### Western Blot

20 μl of TCA precipitated protein was mixed with SDS-PAGE sample loading buffer, boiled for 5 min at 95° C. and electrophoresed on a NuPAGE 4-12% Bis-Tris SDS-gel (Invitrogen). Proteins were transferred to a Hybond-P polyvinylidene difluoride membrane using a Invitrogen Mini Transblot apparatus. The membrane was blocked in PBS-0.05% Tween-20 and 5% skimmed milk (blocking solution) for 1 h at room temperature, and incubated with specific antibodies against Histidine tag (BioRad), 1:800 dilution in blocking solution overnight at 4° C., followed by incubation with peroxidase-conjugated anti-mouse IgG antibody (1:10000) for 1 h at room temperature. The antibody-protein complex was visualized using the ECL Plus Western blotting detection system (GeHealthcare).

Recombinant NGAL Protein Production in E. coli BL21

LCN2 gene coding for NGAL protein was cloned in pETM14 N-terminal his tagged vector and transformed in E. coli BL21 cells. The recombinant human (rhNGAL) protein was expressed in auto-induction LB media o/n at 25° C. Cells were lysed by French press and proteins were purified by a HiTrap Ni2+ column followed by desalting and a size exclusion. From 1 L culture, 98 mg of NGAL protein was obtained.

Toxicity Test of Human Recombinant NGAL Produced in E. coli (rhNGAL) or HEK293 Cells on C. acnes Culture

C. acnes KPA171202 was inoculated to a starting OD600 of 0.05. rhNGAL protein (stock 1.35 mg/mL) produced in E. coli BL21 or HEK293 cells was diluted and added to the cultures at final concentrations of 0, 100, 500 and. 1000 ng/mL Bacteria was incubated with rhNGAL or human NGAL protein at 37° C. degrees, 110 rpm for 24 h. After 24 h toxicity to cultures was measured by their OD600.

### Sebocyte Culture

Immortalized human sebocytes, SZ95 were seeded in a density of 2×10\hat{ }5 cells/well and maintained in DMEM/F12 (Gibco) supplemented with 10% heat inactivated Fetal Bovine Serum (FBS), 100 U/mL Pen/Strep, 1 mM CaCl₂ and 5 ng/ml human epidermal growth factor (EGF; Sigma-Aldrich). Cells were incubated at 37° C. in a humidified 5% CO2 incubator. PCi-SEB_Cau (Phenocell) were seeded and maintained following manufacturer protocols in PhenoCULT-SEB basal medium supplemented with Supplement A ( 1/1000) for 3 days prior to exposure and starting the assays.

MTT Viability Assay of Human Recombinant NGAL Produced in E. coli (rhNGAL) on Human SZ95 Sebocytes

Human SZ95 sebocytes were seeded in a 12-well plate to a density of 2×10\hat{ }5 cells/well and incubated in a humidified atmosphere (37° C., 5% CO2) until 80% confluency in Sebomed media supplemented with 10% heat inactivated FBS, 100 U/mL Pen/Strep, 1 mM CaCl2 and 5 ng/ml human epidermal growth factor (EGF; Sigma-Aldrich Co.). PCi-SEB_Cau (Phenocell) were seeds to 2×10\hat{ }5 cells/well incubated in a humidified atmosphere (37° C., 5% CO2) until 80% confluency in PhenoCULT-SEB basal media supplemented with 1/1000 Supplement A for 3 days prior to treatment. Then, SZ95 were incubated with purified rhNGAL protein produced in E. coli BL21 cells in concentration ranging from 0, 50, 100, 500 or 1000 ng/mL for 24 h and then subsequently incubated with fresh medium and 10% MTT (5 mg/mL in phosphate buffered saline) for 2 h at 37° C. Additionally, SZ95 and PCi-SEB_Cau were incubated with human NGAL protein produced in HEK293 cells (LC2-H5222, ACROBiosystems) in concentrations of 0, 100 and 1000 ng/mL for 96 h and then subsequently incubated with fresh medium and 10% MTT (5 mg/mL in phosphate buffered saline) for 2 h at 37° C. Afterwards, the medium was removed carefully and 500 μL of dimethyl sulfoxide (DMSO) 99% purity was added to lysate the cells and dissolve the purple insoluble crystals of MTT. The cell lysate was transferred to a new 96-well plate and then the absorbance was read using a Microplate Autoreader at excitation/emission of 540/630 nm (Tecan). Absorbance values were considered directly proportional to cell viability.

### Tunnel Assay

SZ95 and PCi-SEB_Cau sebocytes were cultured in 96-well plates as described above. Wells were rinsed with PBS and treated in triplicates with vehicle control, 50 ng/ml, 100 ng/ml, 1 μg/ml of NGAL (E. coli, HEK293 or C. acnes) protein for 24 hours. Samples were prepared by manufacturer's instructions for In Situ Cell Death Detection Kit, Fluorescein (Roche Diagnostics). DNase I treatment of positive and negative controls (without terminal transferase) were included as assay controls according to manufacturer's instructions. Digital images from Operetta HCS fluorescence microscopy experiments were processed using the Harmony high content analysis version 4.9 software package (Perkin-Elmer). When performed, image processing was restricted to changes in brightness and/or contrast and was applied equally across the entire image and different images of the same experiment, including controls. For image analysis, at least three fields per biological replicate were randomly chosen. To measure staining intensity, the Harmony high content analysis software package was used with custom algorithms. The DAPI channel was used to detect nuclei. Cytoplasmic area was determined as a ring of constant size around each nucleus and was used to measure the mean fluorescence intensity of the staining of interest.

### Sebum Modulation Assay

Recombinant NGAL incubation with Pci SEB-Cau sebocytes to measure sebum reduction Pci-SEB_Cau human iPSC derived sebocytes (Phenocell) were seeded in a 24 well plate at cell density of 25000 viable cells/cm² in PhenoCULT SEB-medium supplemented with 1/1000 Supplement A. Cells were grown in a humidified incubator (37° C., 5% CO2) for 3 days before being exposed and starting the assay. Experiment was performed in triplicates being treated with either vehicle, 5 μM AA (arachidonic acid), 5 μM AA+10 μM ISO (13-cis retinoic acid), 5 μM AA+50 ng/mL rhNGAL (from E. coli BL21), 5 uM AA+50 ng/mL pBR13 NGAL (from C. acnes KPA171202, see Table 1), 5 μM AA+50 ng/mL pBR14 NGAL (C. acnes KPA171202, see Table 1), 5 μM AA+50 ng/mL pBR16 NGAL (C. acnes KPA171202, see Table 1). Cells were exposed for 48 h and then cell lipid content measured by BODIPY dye 493/503 (Sigma Aldrich) following manufacturer's recommendations. The nucleus was stained with DAPI. Samples were analyzed by confocal fluorescence microscopy and fluorescence measured and normalized.

### Bacteria Engraftment on Murine Skin

Bacteria (WT and KI) was grown in 20 mL to a OD₆₀₀ of 1, spun down at 1700×g, 10 min and washed 3 times with 0.5% of peptone/PBS and resuspended in 0.5% peptone/PBS to a final volume of 500 ul. Mice (C57BL/6J) were shaved on the back at an area of 2 cm×5 cm. Wild type, knock out or vehicle were applied with a cotton swab on the shaved areas and repeated for consecutive 3 days. Every day before application a sample was taken with a moistened cotton swab and dissolved in 0.5 mL QuickExtract™ DNA Extraction Solution (Lucigen) and treated for 6 min at 65° C. followed by 2 min at 95° C. with 15 seconds vortexing between steps. Samples were taken every 24 h during the full assay period of 7 days. 5 μl DNA extract was used for amplification of the SLST region or 16S V3-V4 region linked to illumina adaptors using KAPA HiFi HotStart Readymix (Roche) (Initial denaturation for 5 min at 95° C. followed by 35 cycles of 98° C. for 20 s, 62° C. for 25 s, and 72° C. 30 s; and a final elongation for 1 min at 72° C.).

SLST_illumina_fwd:
SEQ ID NO: 34
5′-TCGTCGGCAGCGTCAGATGTGTATAAGAGACAGTTGCTCGCAACTG
CAAGCA-3′
and
SLST_illumina_rev:
SEQ ID NO: 35
5′-GTCTCGTGGGCTCGGAGATGTGTATAAGAGACAGCCGGCTGGCAAA
TGAGGCAT-3′
16S_illumina_fwd:
SEQ ID NO: 36
5′-TCGTCGGCAGCGTCAGATGTGTATAAGAGACAGCCTACGGGNGGCW
GCAG-3′
and
16S_illumina_rev:
SEQ ID NO: 37
5′-GTCTCGTGGGCTCGGAGATGTGTATAAGAGACAGGACTACHVGGGT
ATCTAATCC-3′.

Junctions were PCR amplified using KAPA HiFi HotStart Readymix (Roche) (Initial denaturation for 3 min at 95° C. followed by 25 cycles of 98° C. for 20 s, 62° C. for 15 s, and 72° C. 30 s; and a final elongation for 1 min at 72° C.) using primer pairs Junction1_fwd and rev and Junction2_fwd and rev (see Supplementary Table 1).

PCR products were loaded in a 1% agarose gel and visualized by a transilluminator.

### Skin Biopsies

Bacteria was applied to shaved back of mice for 3 consecutive days. 24 h after the last application mice were sacrificed and skin biopsies were taken. Samples for RNA extraction were stored in RNAlater solution while samples for cryosections were frozen embedded in OCD solution.

RNA Extraction and qPCR of Skin Biopsies

Total RNA was extracted from skin (NucleoSpin RNA extraction kit, Macherey-Nagel) according to the manufacturer instructions. RNA was then used for cDNA synthesis (High-Capacity cDNA Reverse Transcription Kit, Thermo Fisher Scientific). Quantitative PCR amplification reactions were performed in the QuantStudio 7 Pro Real-Time PCR System (Applied Biosystems) using TaqMan and Sybr Green Gene Expression Master Mix. Values were normalized to Sdha. The following TaqMan assays (Thermo Fisher Scientific) were used to quantify mRNA expression of mouse Sdha (Mm01352366_m1), II-1β (Mm00434228_m1), II-6 (Mm00446190_m1) and Tnfα (Mm00443258_m1) and primers LCN_qPCR_fwd and rev for detection of human NGAL.

### Gram Staining of Skin Sections

Cryosections from frozen tissue embedded in OCT (Optimal Cutting Temperature (OCT) compound Tissue-Tek) were cut at 7 μm thickness with cryotome. Frozen skin sections were air-dried at room temperature for 15 minutes, fixed with 4% paraformaldehyde for 15 minutes and stained with Gram following standard procedures. Crystal violet was applied for 1 minute following water washing, then iodine for 1 minute then water wash, rapid decolorization was achieved with acetone and a final 1 minute incubation with Safranin with subsequent washes were applied. After drying the samples DPX (Sigma Aldrich) mounting media was applied. Conventional images were captured using a Zeiss apotome microscope.

### Cloning of Plasmids

Plasmids for homologous recombination were cloned as previously described (Sörensen et al. 2010). In short, homology arms were amplified from genomic DNA including restriction sites for NcoI, SpeI and Acc65I. Homology arms were digested with Acc65I and ligated and finally cloned into the pGEM-T-easy vector by restriction-enzyme-mediated cloning. In a second step the gene of interest was cloned between the homology arms in a second step. Replicative plasmids are based on the P. freudenreichii vector pBRESP36A (Jore et al. 2001) and were optimized by Lood et al. for protein production in C. acnes (Lood 2011). Selected secretion peptides and LCN2 genes were cloned downstream of the p1340 promoter using NheI and SpeI restriction sites.

### Minicircle Production

Minicircle strains ZYCY10P3S2T (Kay et al. 2010) (System Biosciences), JMC2 and JMC3 (Johnston et al. 2019) were used to produce minicircles being methylated or lacking endogenous methylation. Gene flanking homology arms were cloned into the parental plasmid pMC BESPx MCS1 (Systems Biosciences) and transformed into ZYCY10P3S2T (ZYCY) (Systems Biosciences), JMC2 or JMC3 minicircle Production Strains. MC production was performed according to the manufacturer's protocol.

Creating a MIIIB Proficient Dam− Dcm− hsdMS− E. coli Strain

To create an E. coli strain that produces plasmids with the C. acnes methylation pattern, lambda-Red recombineering was used as described by Datsenko & Wanner (Datsenko et al. 2000). Briefly, a recipient dam−/dcm− strain (NEB) was first transformed with pKD46 plasmid and selected with 100 μg/ml ampicillin at 30° C. Transformants were grown at 30° C. and induced with 0.4% arabinose for 1 h before transformation with the linear DNA fragment to be recombined into the chromosome (see below). Transformants were selected at 37° C. with 25 μg/ml kanamycin, and successful recombineering was verified by colony PCR and Sanger sequencing. The temperature-dependent loss of pKD46 was confirmed through the absence of growth in Amp 100 μg/ml plates.

The linear DNA fragment to be recombined into the E. coli chromosome was prepared as follows. First, the MIIIB methylase gene was amplified from the genome of C. acnes KPA171202, and it was cloned under Bba_J23100 promoter and Bba_B0034 RBS in a pJET1.2 vector, together with the Tn5 neomycin phosphotransferase (KanR) gene amplified from pKD13 (Datsenko et al. 2000). The region containing the MIIIB methylase and the KanR genes was then amplified with primers PP-19 and PP20, which provide 50 bp homology regions to the E. coli genome. In particular, primers were designed so that recombination occurs in the hsdMS locus, thereby inactivating the hsdMS methylation system upon insertion of the C. acnes methylase gene. To ensure that only the linear PCR product (and not the template plasmid) was transformed, the template plasmid was digested with XhoI and AatII for 1 h at 37° C. prior to the PCR, and then the PCR product was treated with DpnI for 1 h at 37° C. and the correct band was purified from a 1% agarose gel. The template-free, purified PCR product was transformed into dam−/dcm− cells carrying pKD46 as described above.

Transformation of C. acnes:

C. acnes competent cells are prepared as previously described (Meike Sörensen, et al. Mutagenesis of Propionibacterium acnes and analysis of two CAMP factor knock-out mutants, Journal of Microbiological Methods, Volume 83, Issue 2, 2010, Pages 211-216, ISSN 0167-7012, https://doi.org/10.1016/j.mimet.2010.09.008.). Briefly, C. acnes cells are inoculated to a starting OD600 of 0.1 and grown for 24 h anaerobically, 110 rpm at 37° C. degrees. Bacterial culture is centrifuged for 10 min, 4200 rpm and washed 7 times in total with ice-cold 272 mM Sucrose buffer. 10 μl of bacterial pellet is diluted in 30 μl of 272 mM Sucrose buffer and mixed with 8 μg methylated plasmid DNA. Methylated plasmid DNA was produced by shuttling the plasmid through a IIIB methylation proficient E. coli dam−dcm− strain (see above). This E. coli strain was engineered to produce the C. acnes IIIB methylase. The bacteria were transformed with the plasmid DNA by electroporation using a Biorad gene pulser apparatus (BioRad) at 1500V, 400 ohm, 25 uF, 1 mm. Cells were recovered for 24 h on a Brucella agar plate anaerobically and afterwards selected on a Brucella plate supplemented with 10 μg/mL erythromycin. Plates were incubated anaerobically for 7 days at 37° C. degrees. Positive clones were checked by junction PCR and WGS for positive gene insertion/plasmid transformation.

To improve electroporation efficiency different pre-treatments were tested. For glycine or penicillinG pre-treatment, cells were grown in presence or absence of 1.25% of glycine or 2, 5, 10 μg/mL PenicillinG, 4 h prior to electroporation. For osmotic stabilization 0.4 M of sucrose was added also 4 h prior.

Inhibiting Thymidine Kinase (Tdk) in Wild Type C. acnes for a Potential Negative Selection

To test if tdk could be used as a negative selection in C. acnes, we tested its functionality with the known inhibitor 5-fluoro-2′-deoxyuridine (FUDR). We plated serial dilutions of C. acnes wild type KPA171202 from 10\hat{ }-1 to 10\hat{ }-8 in 10 μl drops on Brucella agar plates containing 0 or 50 μg/mL FUDR. Plates were incubated for 7 days at 37° C. anaerobically and checked for potential growth. If no growth was seen, the inhibitor FUDR is functional against the tdk gene.

### Thymidine Kinase Knock Out Functional Testing

The tdk knock-out was created as previously described in Sörensen et al. 2010 targeting the thymidine kinase locus and cloning the pMW535 plasmid with homologous arms to the region. For simplicity the antibiotic resistance cassette, coding for erythromycin (ermE) was cloned between the homology arms to replace the tdk gene with an ermE cassette. To evaluate the functionality of the tdk dual selection marker (Shao et al. 2016; Norville et al. 2016), we tested the knock-in in sensitivity to FUDR (50 ug/mL) and erythromycin (10 ug/mL) and compared it to the wild type. Therefore, we plated bacteria on Brucella agar plates either containing ery (10 ug/mL), FUDR (50 ug/mL) or neither of the components. Only the knock-in strain could grow on both ery and FUDR containing plates, while the wild type was sensitive to both.

### Example 2: Results

Viability Test of SZ95 and PCi-Seb_Cau Human Sebocytes and C. acnes Treated with rhNGAL

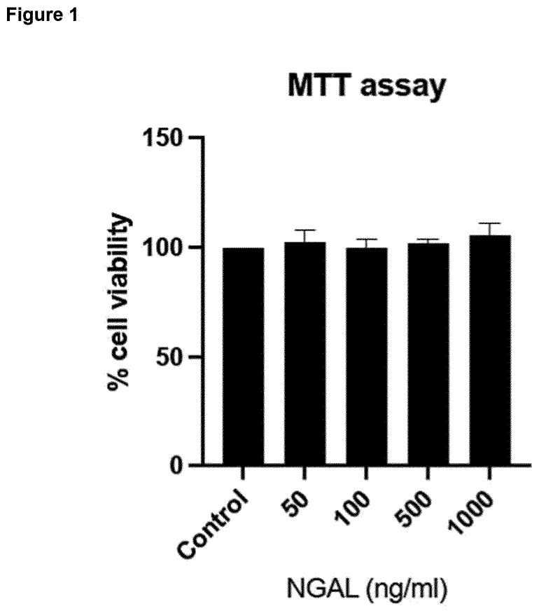

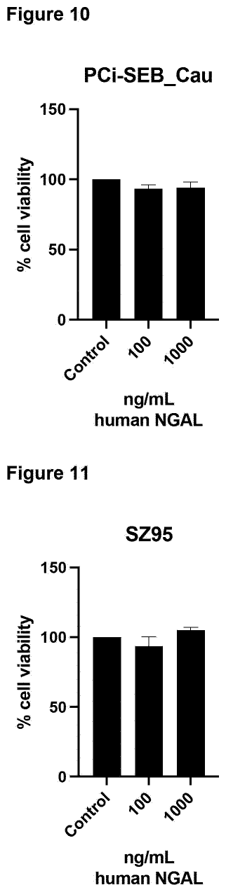

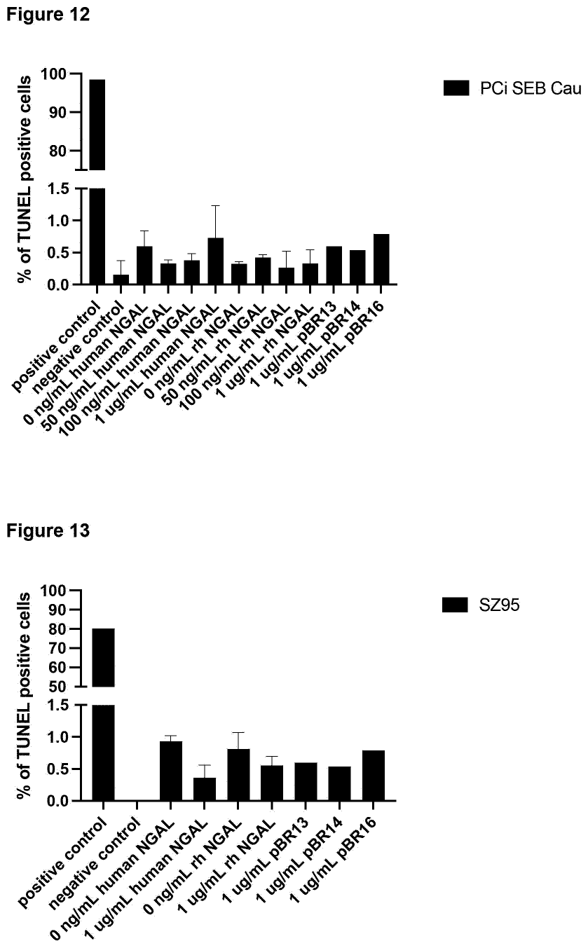

To investigate the effects of rhNGAL protein produced in E. coli or HEK293 cells on the proliferation of human sebocytes, we used the human SZ95 and PCi-Seb_Cau sebocyte cell line. In the present study, the antiproliferative effect of NGAL on sebocytes was determined. Therefore, cells were treated with various concentrations of human NGAL (produced in HEK293 cells) and cell proliferation and viability assessed by a MTT (colorimetric assay for assessing cell metabolic activity) assay. No significant effect was found after 24 h on the viability and proliferation of the SZ95 and PCi-Seb_Cau cells in a dose dependent manner (FIG. 1, FIGS. 10 and 11)

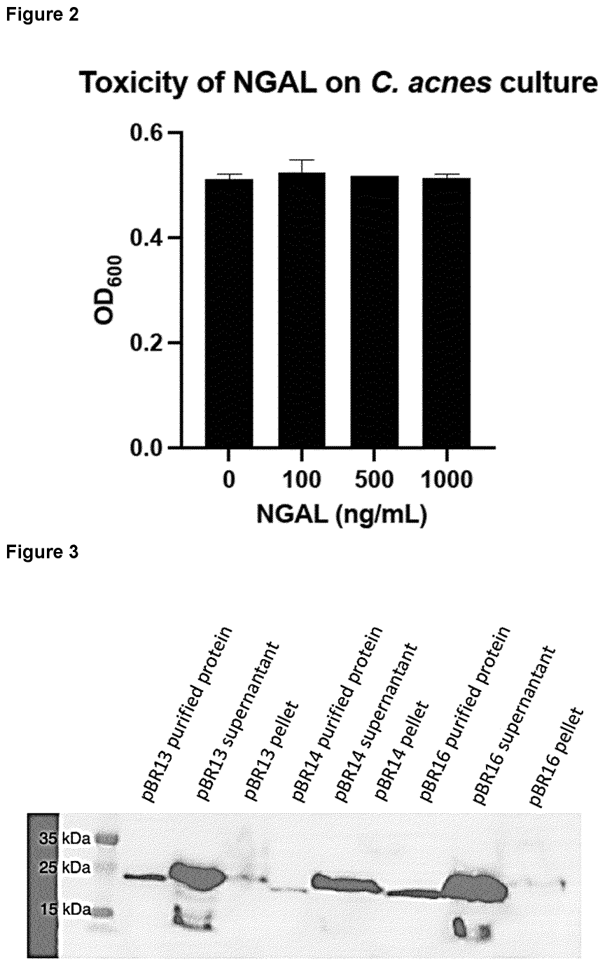

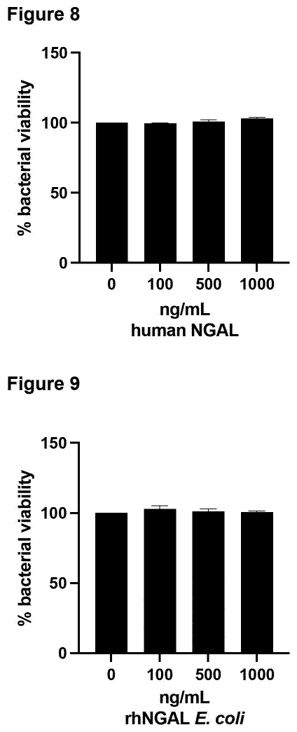

As another test, we assessed rhNGAL (E. coli) and human NGAL (HEK293) toxicity on C. acnes culture using several doses. OD600 was measured after 24 h incubation and no toxic effect observed on C. acnes bacteria measured on its growth comparing to the un-treated control (FIG. 2, FIG. 8, FIG. 9). The used rhNGAL protein was expressed and purified from E. coli BL21 cells (see Example 1 above) and the human NGAL was produced in HEK293 cells and commercially acquired from ACROBiosystems, see FIG. 8 and FIG. 9

Protein Production and Secretion of NGAL in C. acnes

Total protein production of NGAL protein was measured of the three different constructs pBR13, pBR14 and pBR16 (see Table 1) in the secreted fraction vs. cytosolic fraction of the TCA precipitated protein. Secreted and cytosolic fractions were visualized by Western blot and compared to 40 μg (pBR13), 10 μg (pBR14) and 25 μg (pBR16) of purified protein. Western blot showed higher amount of protein in the secreted fraction (FIG. 3).

Western Blot of NGAL Produced in E. coli, C. Acnes or HEK293

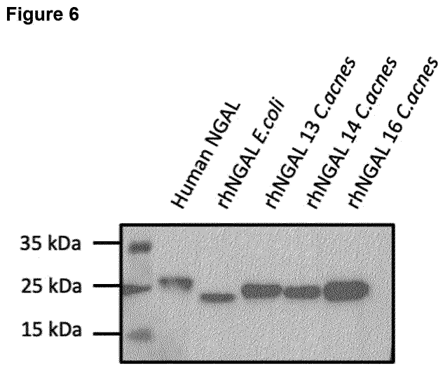

Equal amounts of purified proteins were loaded on a SDS gel followed by Western blotting and detection with an anti-histidine antibody. HEK293 cells show higher molecular weight due to glycosylation of the human protein. E. coli derived NGAL has the lowest molecular weight due to no glycosylation. C. acnes produced NGAL seems to have some posttranslational modification due to the increased molecular weight in comparison to the E. coli produced protein (FIG. 6).

rhNGAL Reduces Sebum Production in PCi-SEB Cau Sebocytes In Vitro

To test the ability of purified rhNGAL produced in E. coli or C. acnes in reducing sebum in PCi-SEB_Cau we induced sebum production with 5 μM Arachidonic acid (AA) and treated them simultaneously with rhNGAL and isotretinoin for 48 h. It could be seen, that all NGAL proteins, i.e., rhNGAL produced in E. coli (1.7-fold), pBR13 (2.5-fold), pBR14 (1.5-fold), pBR16 (1.6-fold) and isotretinoin (1.8-fold) induced a significant decrease in sebum production compared to AA treated cells (FIG. 4). Interestingly, the NGAL protein produced in C. acnes (pBR13) significantly reduced sebum 1.3-fold in comparison to the positive control isotretinoin (FIG. 4).

### TDK Test

We checked the ability of growth of C. acnes KPA171202 wild type strain on Brucella agar plates, supplemented with or without 50 μg/mL FUDR (5-fluoro-2′-deoxyuridine). The results (FIG. 5), show that bacteria could only grow in absence of the thymidilate synthase inhibitor (FUDR), showing that that the inhibitor is functional against the thymidine kinase. These results shown that a thymidine kinase (tdk) knock out could be used to select for our LCN2 insertion by using the inhibitor and therefore not require an antibiotic resistance. The absence of antibiotic resistance is important for the application of the bacteria on the human skin.

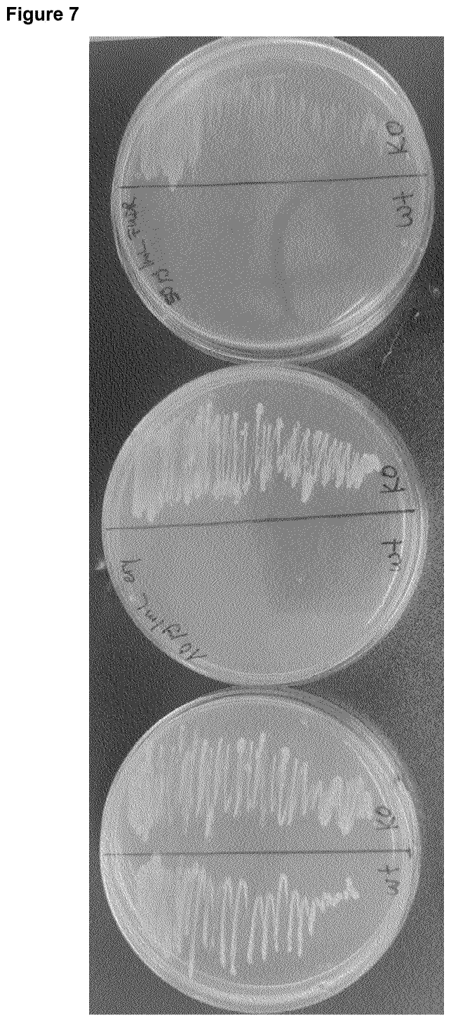

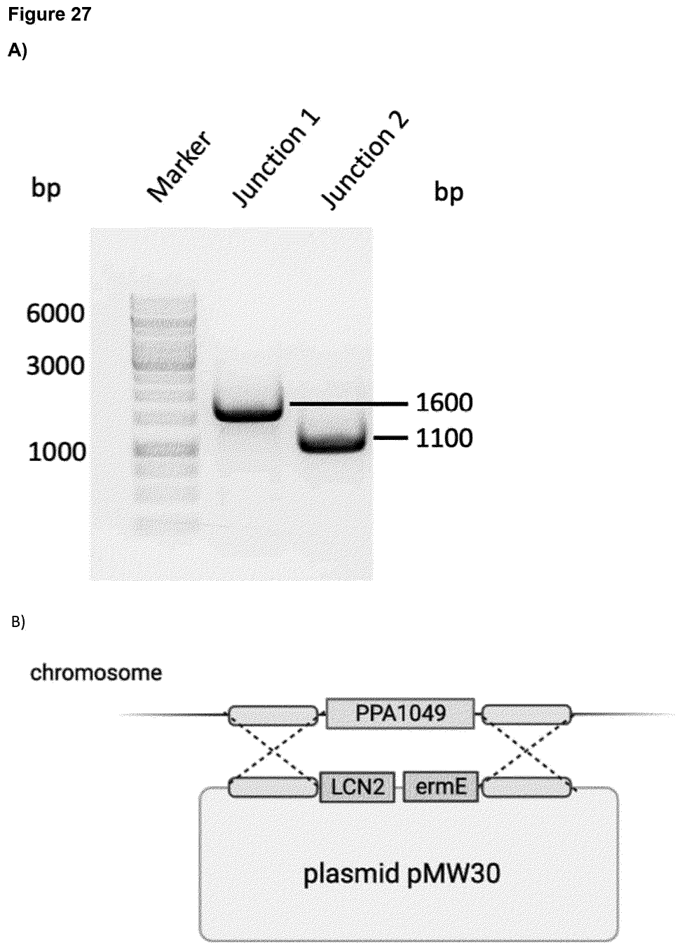

After creating the tdk knock-out by homologous recombination (FIGS. 27a and b), we tested its functionality by measuring its sensitivity or resistance to FUDR. While wild type bacteria could not grow on FUDR (50 ug/mL), the tdk knock-out survived and no growth inhibiting effect was seen (FIG. 7).

### Engraftment of Bacteria on Mice

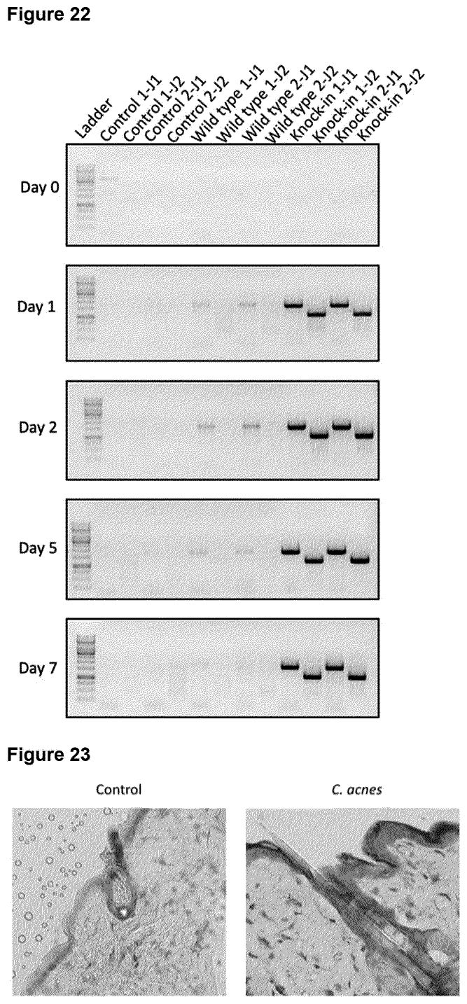

Finally, we wanted to test if our sebum-modulating strain could engraft efficiently on skin. Therefore, we applied both the wild type and the engineered C. acnes on the back of mice for three consecutive days and continued sampling until day 7. We followed engraftment by junction PCR and confirmed the presence of the engineered strain until assay endpoint at day 7 (FIG. 22). We conducted gram-staining of cryosections of the skin and observed an increase in gram-positive bacteria on the skin and in the hair follicles of treated mice. The gram-staining observed in the control mice, which was quantitatively much less abundant than in treated mice, probably reveals the natural flora (FIG. 23).

### Protein Production In Vivo

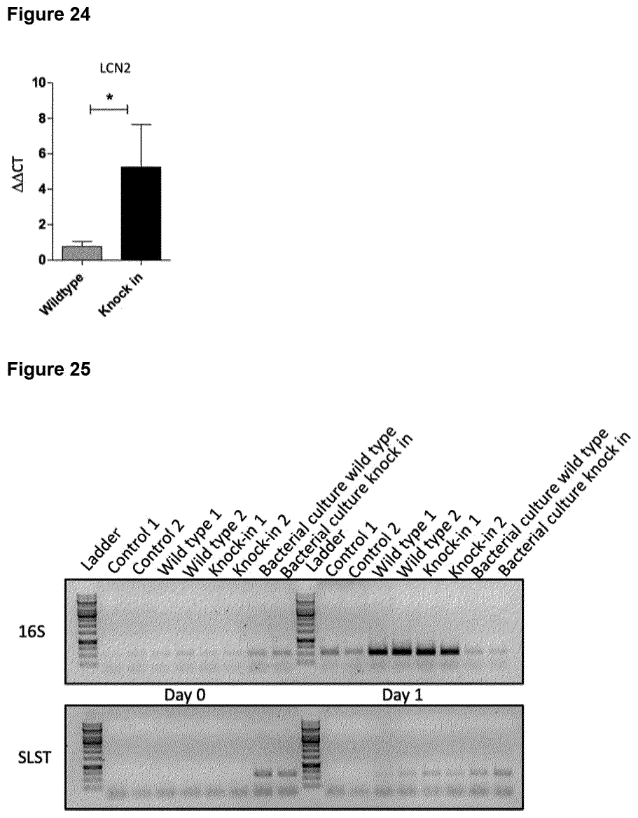

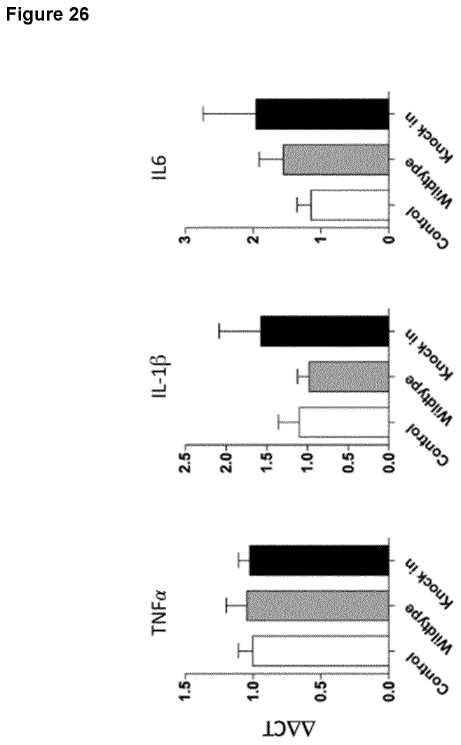

Since bacteria seemed to colonize on murine skin, in vivo protein production was assessed. qPCR analysis revealed increased expression of NGAL in samples treated with the engineered bacteria whereas wild type and vehicle-treated murine skin showed limited expression (FIG. 24). To investigate potential inflammation derived from C. acnes treatment, we assessed expression levels of inflammatory cytokines IL1-β, IL-6 and TNFα. No differential expression of inflammatory cytokines was observed when comparing treated and untreated samples (FIG. 26).

### Improving Transformation Efficiency

We screened various transformation buffer compositions and conditions, which were successfully used in other non-model organisms. Since electro transformation efficiency seemed to be very low in general, we used a fluorescently labeled oligonucleotide to assess the entry and stability of DNA in C. acnes cells. DNA entry of the fluorescent oligo increased around 50-fold using a buffer containing 272 mM sucrose compared to other electroporation buffers tested (FIG. 14). We observed a significant reduction in fluorescence after 1 h of recovery post-electroporation and concluded that the fluorescent oligo must have been partially degraded (FIG. 15).

### Discussion of the Results Regarding a Prokaryote Expressing NGAL

In this study, we assessed the effect of bacterial NGAL on human sebocytes. The protein to be tested was produced in E. coli BL21 or C. acnes KPA171202. Three different plasmid versions of NGAL with combinations of regulatory parts (pBR13, pBR14 and pBR16) were transformed in C. acnes and tested for the ability in producing and secreting NGAL protein. We verified the presence of NGAL protein in C. acnes supernatant by protein purification and western blot and showed that pBR13, pBR14 and pBR16 were able to secrete NGAL, whereas an increased production was observed in pBR13 and pBR16.

Additionally, NGAL protein had no effect on the viability of C. acnes and on human SZ95 sebocytes in vitro, which is contrary to what is has been previously shown (Klausen et al. 2005. On mouse and man: neutrophil gelatinase associated lipocalin is not involved in apoptosis or acute response. Eur J Haematol: 75: 332-340 and Kimberly Ruth Lumsden. 2011. The Innate Immune Protein Neutrophil Gelatinase-Associated Lipocalin Is Involved in The Early Therapeutic Response To 13-Cis Retinoic Acid In Acne Patients. PhD Dissertation).

We also measured ability of bacterial NGAL in reducing sebum from stimulated PCi SEB-Cau sebocytes in vitro. A significant reduction in sebum was observed after 48 h which was comparable to isotretinoin treatment. The C. acnes produced NGAL (pBR13) showed a 1.3-fold decrease in sebum compared to isotretinoin treatment.

In this study we demonstrate that C. acnes can produce therapeutic amounts of NGAL and is able to secrete the protein to the supernatant. Filtered supernatant and purified protein of C. acnes was able to reduce sebum comparably to isotretinoin in an in vitro sebocytes model. This data suggests the beneficial use of NGAL produced in bacteria (E. coli and C. acnes), together with the potential therapeutic use of NGAL producing C. acnes as a shuttle for cosmetic purposes or to reduce sebum and treat acne vulgaris in a subject in need thereof. This treatment could be a safe and efficient alternative to isotretinoin which exhibits many severe side-effects.

Furthermore, upon application on skin, the skin microbe colonizes the hair follicle and shows persistence for the complete study period while producing and secreting a therapeutic which can modulate sebum production while targeting and interacting with the sebaceous gland. This study presents evidence for the controlled use of the skin commensal through a dual selection based on the thymidine kinase gene. The basis of this strain will support future clinical studies and its use in humans while providing the basis for regulatory measures.

### Discussion of the Results Regarding a Transformation Method:

Using live biotherapeutics as a new class of medicine has both great potential and advantage over conventional medicine and can fulfill unmet needs. Multiple engineered live biotherapeutics are currently under development and are entering clinical development which are mainly focused on the gut.

The skin represents another great potential target for the application and development of live probiotics, considering its easy accessibility and its important functions for human health. The skin is the primary barrier to the human body and is exposed to many external harsh conditions like UV irradiation, pollution and pathogens. Therefore the development of a skin probiotic can protect the human from harmful environmental conditions and sense early onset of damage and disease.

We show the development and efficient engineering of the skin commensal Cutibacterium acnes by improving various steps in the electroporation of the bacterium. We show the importance of mimicking the host's own methylation pattern to avoid induced endogenous degradation of transformed nucleic acids into C. acnes. In this method, the nucleic acid to be introduced in C. acnes is characterized by completely lacking methylated nucleobases and by recoding REase recognized patterns, or by only presenting methylated nucleobase that were methylated by a C. acnes methylase, and thus are recognised by “self” upon delivery into C. acnes. Shuttle bacteria, such as E. coli, may be used to provide the nucleic acid before its introduction in C. acnes. However, said E. coli needs to be modified to have its endogenous system fully silenced (dam− dcm− hsdMS− E. coli), and preferably to also express a C. acnes methylase. This differs with other previously described methods where the shuttle E. coli only had one gene silenced, such as dam− E. coli, or did not express C. acnes methylase. The complete removal of the E. coli endogenous methylation system improves the delivery efficiency of the plasmid into C. acnes and the expression of the own C. acnes methylation systems improves the efficiency even further.

Therefore, we developed a E. coli strain that lacks its own endogenous methylation systems but mimics C. acnes methylation by expressing the C. acnes IIIB methylase. This protects foreign nucleic acids from being detected by C. acnes and will be maintained by the cell. Furthermore, treatment of cells during competent cell preparation with cell-wall-permeabilizing agents like PenicillinG or L-Glycine increased transformation efficiency. Also, lower DNA amounts, higher OD and the use of frozen competent cells were favorable for the transformation outcome. Homologous recombination could also be increased by using minicircle vectors the gene of interest recoded to remove C. acnes recognizable pattern or by using minicircle producer strains with silenced endogenous methylation systems.

1. A method for introducing a nucleic acid into Cutibacterium acnes (C. acnes), the method comprising the steps of:
a) providing an unmethylated nucleic acid with the proviso that if it comprises a C. acnes methylation motif, said methylated motif is methylated, and
b) introducing said nucleic acid from step a) into C. acnes.

2. The method according to claim 1, wherein C. acnes methylation motif that is methylated in step a) is AGC(m)AGY.

3. The method according to any one of claims 1 or 2, wherein the C. acnes of step b) is obtained by:
i) having been previously cultured in the presence of at least one permeabilizing agent of bacterial cell wall, and/or
ii) having been previously frozen and thawed.

4. The method according to claim 3, wherein the permeabilizing agent of bacterial cell wall is L-glycine or penicillinG.

5. The method according to any of claims 1 to 4 wherein the nucleic acid sequence of step a) comprises one or more methylated C. acnes methylation motifs in its sequence, wherein said nucleic acid is obtained by introducing a nucleic acid comprising one or more C. acnes methylation motifs in its sequence into a bacterium that is not C. acnes, wherein the bacterium that is not C. acnes is characterized by comprising or expressing a C. acnes methylase and by not comprising or expressing any other methylase, and subsequently collecting said nucleic acid from said bacteria to proceed with step b).

6. The method according to claim 5, wherein the C. acnes methylase is a C. acnes IIIB methylase.

7. The method according to any of claims 1 to 4 wherein the nucleic acid sequence of step a) is unmethylated and does not comprise any C. acnes methylation motifs in its sequence, wherein said nucleic acid sequence is obtained by introducing a nucleic acid that does not comprise any C. acnes methylation motifs in its sequence into a bacterium that is not C. acnes, wherein the bacterium that is not C. acnes is characterized by not expressing any other methylase, and subsequently collecting said nucleic acid from said bacterium to proceed with step b).

8. The method according to any one of the claims 5 to 7, wherein the bacterium that is not C. acnes is a dam− dcm− hsdMS− E. coli strain.

9. The method according to any one of the claims 5 to 8, wherein the bacterium that is not C. acnes is the E. coli strain deposited in the Spanish Collection of Type Cultures with CECT No. 30749.

10. The method according to any one of the previous claims, wherein the nucleic acid comprises C. acnes integration elements that lead the integration of said target nucleic acid or at least a portion thereof in the genome of C. acnes.

11. The method according to any of the previous claims, wherein the C. acnes that have been transformed are selected, and wherein the selection is based on at least two bacterial selection markers.

12. A recombinant C. acnes obtained by the method of claims 1 to 11.

13. The recombinant C. acnes according to claim 12, wherein the nucleic acid encodes for the human neutrophil gelatinase-associated lipocalin (NGAL) gene and wherein the recombinant C. acnes is characterized in that it expresses, and preferably secretes, NGAL protein.

14. A recombinant C. acnes according to claims 12 or 13, for use in therapy.

15. A recombinant C. acnes according to claims 12 or 13, for use in diagnosis.

16. Use of the C. acnes of claims 12 or 13, in cosmetics.

17. A recombinant Cutibacterium acnes characterized in that it expresses the human neutrophil gelatinase-associated lipocalin (NGAL) protein.

18. The recombinant Cutibacterium acnes according to claim 17, wherein the human neutrophil gelatinase-associated lipocalin (NGAL) protein has at least 85% amino acid sequence identity over the full length to SEQ ID NO: 1.

19. The recombinant Cutibacterium acnes according to any one of the claims 17 or 18, wherein the nucleic acid encoding for the human neutrophil gelatinase-associated lipocalin (NGAL) is operably linked to a promoter selected from the list consisting of camp2 promoter, camp1 promoter or roxP promoter.

20. A recombinant Cutibacterium acnes according to any one of the claims 17 to 19, for use in the treatment of acne vulgaris, urticaria, eczema, rosacea, Hidradenitis suppurativa and/or psoriasis.

21. A non-therapeutical cosmetic use of the recombinant Cutibacterium acnes according to any one of claims 17 to 19, wherein the use is preferably to prevent, reduce and/or ameliorate skin aging.

22. A recombinant Cutibacterium acnes for use according to claims 20 or 21, wherein the use comprises the topic administration of the recombinant C. acnes into the skin.

23. A method for producing the active ingredient of a pharmaceutical composition, wherein said active ingredient is a human neutrophil gelatinase-associated lipocalin (NGAL) protein, wherein the method comprises culturing the recombinant Cutibacterium acnes defined in any of claims 17 to 19.

24. The method according to claim 23, wherein the human neutrophil gelatinase-associated lipocalin (NGAL) protein has at least 85% amino acid sequence identity over the full length to SEQ ID NO: 1.

25. The method according to any of claims 23 or 24, wherein the recombinant bacterium is Cutibacterium acnes and wherein the human neutrophil gelatinase-associated lipocalin (NGAL) protein has at least 85% amino acid sequence identity over the full length to SEQ ID NO: 1.


SEQUENCE LISTING (40 sequences)

SEQ ID NO: 1 - protein, 178 aa, synthetic construct
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 2 - DNA, 534 bp, synthetic construct
  source 1..534: mol_type = other DNA
caggactccacctccgacctcatcccggccccgccgctctccaaggtcccgctccagcagaacttccaggacaaccagttccagggcaagtggtacgtcgtcggcctcgccggcaacgccatcctccgcgaggacaaggacccgcagaagatgtacgccaccatctacgagctcaaggaggacaagtcctacaacgtcacctccgtcctcttccgcaagaagaagtgcgactactggatccgcaccttcgtcccgggctgccagccgggcgagttcaccctcggcaacatcaagtcctacccgggcctcacctcctacctcgtccgcgtcgtctccaccaactacaaccagcacgccatggtcttcttcaagaaggtctcccagaaccgcgagtacttcaagatcaccctctacggccgcaccaaggagctcacctccgagctcaaggagaacttcatccgcttctccaagtccctcggcctcccggagaaccacatcgtcttcccggtcccgatcgaccagtgcatcgacggc

SEQ ID NO: 3 - DNA, 69 bp, synthetic construct
  source 1..69: mol_type = other DNA
atgttcgtccaaatcgctgccagcctggcagccgcatcgtccattgcactcggcataccaggagctgcc

SEQ ID NO: 4 - DNA, 84 bp, synthetic construct
  source 1..84: mol_type = other DNA
atgaagaagacccatcttgtagctcccctccttgtcggcgcaatgctcgtaccagcggcgctgtcagctcccagtgctcatgct

SEQ ID NO: 5 - DNA, 84 bp, synthetic construct
  source 1..84: mol_type = other DNA
atgaaggttaagttcttagcagcgccgctggttgttggtgccttgatggcgccggcagctttctctggagcgacagctcatgct

SEQ ID NO: 6 - protein, 23 aa, synthetic construct
MFVQIAASLAAASSIALGIPGAA

SEQ ID NO: 7 - protein, 28 aa, synthetic construct
MKKTHLVAPLLVGAMLVPAALSAPSAHA

SEQ ID NO: 8 - protein, 28 aa, synthetic construct
MKVKFLAAPLVVGALMAPAAFSGATAHA

SEQ ID NO: 9 - protein, 201 aa, synthetic construct
MFVQIAASLAAASSIALGIPGAAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 10 - protein, 206 aa, synthetic construct
MKKTHLVAPLLVGAMLVPAALSAPSAHAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 11 - protein, 206 aa, synthetic construct
MKVKFLAAPLVVGALMAPAAFSGATAHAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 12 - DNA, 260 bp, synthetic construct
  source 1..260: mol_type = other DNA
taggttgactggaaaggggatatgacacccatcgtcctgggtctgacaggggtagacaatatagcttagaaatgtgtataagtaagatgtttctcgcaatgaaagacgatatgtcacgagttttctggtgctgtttcccgacacggaaccgttagtttgaatcaattttgaactgaggacaccaagttgaccccctggtgtgcaacgatgcttcttgcttgcagttgcgagcaattgttcccgatgaaaggaacccacaa

SEQ ID NO: 13 - DNA, 209 bp, synthetic construct
  source 1..209: mol_type = other DNA
acaccgacgatctgcacgggcgagtctaggggttatcggctacatgcgcgtcgccgcatcaggttcacgaaaccgaaaggcagacccaccccctaccgggcataccgttgccgccgacacttcgatgacactatgcagtgctacacttcaactgaaaatctatatgattttcgtcaattcccatacgtacgacacatcgggagatcgtc

SEQ ID NO: 14 - DNA, 200 bp, synthetic construct
  source 1..200: mol_type = other DNA
gcaacaagcctagacgggtctatgcaccgtcgtagccatacaccacacgggcccctgtcggaatgacgcacgcctgatcacagctccaacaaatgttggcggcgtacacgccgttcaccacaccgacccccatccaaaatatccgatacacttcgcttgcctcgaccggggcatccgcaaccaataaacggagaaccttt

SEQ ID NO: 15 - DNA, 5139 bp, synthetic construct
  source 1..5139: mol_type = other DNA
ttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatggcggccgcgggaattcgcccttccggccgccatggcgaaggatggcagtcggcagcatggagatcgcgcctcatgtgggttgacgagctcgttggtgctcttctcgatgagctcgatcctggaaccgccctggtcgtcactgctgatcacggcatggttgacatccccgacgaccaccgcgtcatggtggaggacgagggtggactgctcgctgacgttgccttgttggctggtgagcctcggttccgccacatttacactcgtcgtcccgatgaggtcgcgcaacgatggcgacgcgtactcggggatcgtgctcaggttgtcattggcgacgatgctgttgcggctgacctgtttggaccagttgacgtgtctacccgttcccgtttcggtgacgtactagcgattactcgtggtgattgggccatcatgagccgcgctacccccaaggaactcaccctcgtgggcatgcacggatcgctcactccgtgtgaaatgcggatcccattgctggcccagatctgtgaaggatgaggggtacctaggttgactggaaaggggatatgacacccatcgtcctgggtctgacaggggtagacaatatagcttagaaatgtgtataagtaagatgtttctcgcaatgaaagacgatatgtcacgagttttctggtgctgtttcccgacacggaaccgttagtttgaatcaattttgaactgaggacaccaagttgaccccctggtgtgcaacgatgcttcttgcttgcagttgcgagcaattgttcccgatgaaaggaacccacaaatggctagcatgttcgtccaaatcgctgccagcctggcagccgcatcgtccattgcactcggcataccaggagctgcccaggactccacctccgacctcatcccggccccgccgctctccaaggtcccgctccagcagaacttccaggacaaccagttccagggcaagtggtacgtcgtcggcctcgccggcaacgccatcctccgcgaggacaaggacccgcagaagatgtacgccaccatctacgagctcaaggaggacaagtcctacaacgtcacctccgtcctcttccgcaagaagaagtgcgactactggatccgcaccttcgtcccgggctgccagccgggcgagttcaccctcggcaacatcaagtcctacccgggcctcacctcctacctcgtccgcgtcgtctccaccaactacaaccagcacgccatggtcttcttcaagaaggtctcccagaaccgcgagtacttcaagatcaccctctacggccgcaccaaggagctcacctccgagctcaaggagaacttcatccgcttctccaagtccctcggcctcccggagaaccacatcgtcttcccggtcccgatcgaccagtgcatcgacggctctagaatcgaaggtcgtggcaagccgatcccgaacccgctcctcggcctcgactccacccgcaccggccaccaccatcaccaccattagtcgagcccggaggagaagagactgtgaacacgaacatgggcccacccctcaagaggaatgggcccatgccgtaaatgatttcgatgcaagtgtcaggacaacagcagggtaccatggctccaggtcctgacgagcgtggccccagggttcgcgtgcggtcaaggattttcccgcggtgggtggtatatcaggtatgcggggtggggccgccgaacatgatctcatcccaagaagggatcgacgcccgcttcgatttacgcggtttgcgcttcttgcgggggcggctaccctcctcgacgagggcgtcctgctggggagtctcatcggcagcttgagctgacgcttgtgagcgagaagtcgaagtcgaggtcttcttcgtcgatgtgcgtagcttcgacgccgcagtatcctgcgcggaggtgtcgtcatggggtggctgggcctcgcttttccccccagcctgatctggttgggatgtctcgatggcatgcggagccccggctggctggggagcctggggcgtagctggctcaggatcctgattcacctccggatgagacgcagcgtcaaggactggggtggcgagaccttcatagatgttgaccgagtcctcttgaaacccggcaagcacactagtgaattaagggcgaattcgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattg

SEQ ID NO: 16 - DNA, 9215 bp, synthetic construct
  source 1..9215: mol_type = other DNA
gaattcaagcttgtcgacgttaacctgcaggcatgcggatccggtaccagcccgacccgagcacgcgccggcacgcctggtcgatgtcggaccggagttcgaggtacgcggcttgcaggtccaggaaggggacgtccatgcgagtgtccgttcgagtggcggcttgcgcccgatgctagtcgcggttgatcggcgatcgcaggtgcacgcggtcgatcttgacggctggcgagaggtgcggggaggatctgaccgacgcggtccacacgtggcaccgcgatgctgttgtgggctggacaatcgtgccggttggtaggatccagcggtgagcagttcggacgagcagccgcgcccgcgtcgccgcaaccaggatcggcagcaccccaaccagaaccggccggtgctgggccgtaccgagcgggaccgcaaccggcgccagttcgggcagaacttcctccgcgaccgcaagaccatcgcgcgcatcgccgagacagccgagctgcggcccgatctgccggtgctggaagccggccccgtcgaagggctgctcaccagggaactcgccgaccgcgcgcgtcaggtgacgtcgtacgagatcgacccccggctggcgaagtcgttgcgggagaagctttccggccacccgaacatcgaagtcgtcaacgccgacttcctcaccgccgaaccgccgcccgagccgttcgccttcgtcggcgcgatcccctacggcatcacctcggcgatcgtggactggtgcctggaggcgccgacgatcgagacggcgacgatggtcacgcagctggagttcgcccggaagcggaccggcgattacggccgctggagccgcctcacggtgatgacctggccgctgttcgagtgggagttcgtcgagaaggtcgaccgccggctgttcaagccggtgcccaaggtcgactcggcgatcatgcggctgcgcaggcgcgccgaaccgctgctggaaggcgcggcgctcgaacgctacgagtcgatggtcgagctgtgcttcaccggcgtcggcggcaacatccaggcgtcgcttctgcgcaagtacccgaggcgccgcgtcgaggcggcgttcgaccacgcgggggtcgggggcggcgccgtggtcgcctacgtccggccggagcagtggctccggctgttcgagcggctggatcagaagaacgaaccgaggggtgggcagccccagcggggcaggcgaaccggcggacgggaccacggggaccggcgaaccggcgggcaggatcgcggcgatcggcgaaccggcggccgcgaccacagggaccggcaagccagcggccacggcgatcgtcgcagcagcggacgcaatcgcgacgacggacgaaccggcgagcgcgagcagggggaccaaggcgggcggcgggggccgtccgggggtggacggaccggcggacgtccagggcgacgcggcggacccgggcagcggtagtccccggcacgcggaacgcggcaggccgtcgagcggcctgccccgttctgtcgagaggaatcagaggttgatgtcggcccggaggtcgatgtcgcgcgacgacgagccgatctccaccgctcgcttgccgcccccgagcttccagccgcccgcggcttcgtcccagtgctggagggccgctccgcgacgtgcacccggacgcgcttggtctcgcccggtgcgagttcgaccttctggtacctaggttgactggaaaggggatatgacacccatcgtcctgggtctgacaggggtagacaatatagcttagaaatgtgtataagtaagatgtttctcgcaatgaaagacgatatgtcacgagttttctggtgctgtttcccgacacggaaccgttagtttgaatcaattttgaactgaggacaccaagttgaccccctggtgtgcaacgatgcttcttgcttgcagttgcgagcaattgttcccgatgaaaggaacccacaaatggctagcatgttcgtccaaatcgctgccagcctggcagccgcatcgtccattgcactcggcataccaggagctgcccaggactccacctccgacctcatcccggccccgccgctctccaaggtcccgctccagcagaacttccaggacaaccagttccagggcaagtggtacgtcgtcggcctcgccggcaacgccatcctccgcgaggacaaggacccgcagaagatgtacgccaccatctacgagctcaaggaggacaagtcctacaacgtcacctccgtcctcttccgcaagaagaagtgcgactactggatccgcaccttcgtcccgggctgccagccgggcgagttcaccctcggcaacatcaagtcctacccgggcctcacctcctacctcgtccgcgtcgtctccaccaactacaaccagcacgccatggtcttcttcaagaaggtctcccagaaccgcgagtacttcaagatcaccctctacggccgcaccaaggagctcacctccgagctcaaggagaacttcatccgcttctccaagtccctcggcctcccggagaaccacatcgtcttcccggtcccgatcgaccagtgcatcgacggctctagaatcgaaggtcgtggcaagccgatcccgaacccgctcctcggcctcgactccacccgcaccggccaccaccatcaccaccattagtcgagcccggaggagaagagactgtgaacacgaacatgggcccacccctcaagaggaatgggcccatgccgtaaatgatttcgatgcaagtgtcaggacaacagcaggatcatccacgcaatgaaagcccgcacccaatacctcgaccagctctaaccaagaaaggaacctgatgagcgaccagctagacagcgaccgcaactacgacccgatgatcttcgacgtgatgcgcgagaccgcgaaccgcgtcgtcgccacgtacgttgcatgggaagatgaagccgctgatccccgcgaggctgcgcactggcaggccgagcgattccgcacccggcacgaggtgcgcgccgtcgaccctgacagccggcgagcagttcaggcgaagatcgcacagctgcgcgaggaactagccgcaatgcccgaacacgccccagccatcccttggagcaggtggcagcgtcaggggagtcgggggatgtttggcaggggatgtggaaagagagttcgctttgctcacatggctcaaccgggtaactaactgatatggggtcttcgtcgcccactttgaacacgccgaggaatggaccacgctgaacgtgactcgcatgcttcactgcatgtatggattcgttcgagacgttgttccctgagagctggctgccacgcaagccgctggcgtcagccgagaagtctggggcgtaccggcacgtgactcggcagagggcgctggagctgccttacatcgaagcgaacccgttggtcatgcagtccttggtcatcaccgatcgagatgcttcggatgctgactgggccgcagacctcgctgggctgccttcaccgtcctacgtgtccatgaaccgtgtcacgaccaccggacacatcgtctatgccttgaagaaccctgtgtgtctgaccgatgccgcgcggcgacggcctatcaacctgctcgcccgcgtcgagcagggcctatgcgacgttctcggcggcgatgcatcctacgggcaccggatcacaaagaacccgctcagcaccgcccatgcgaccctctggggccccgcagacgcgctctacgagctgcgcgccctcgcacacaccctcgacgagatccacgcactgccggaggcagggaacccgcgtcgcaacgtcacccgatcaacggtcggccgcaacgtcaccctgttcgacaccacccgcatgtgggcataccgggccgtccggcactcctggggcggcccggtcgccgaatgggagcacaccgtattcgagcacatccacctactgaacgagacgatcatcgccgacgaattcgccacaggccccctcggcttgaacgaacttaagcacttatctcgatccatttcccgatgggtctggcgcaacttcacccccgaaaccttccgcgcacgccagaaagcgatcagcctccgtggagcatccaaaggcggcaaagaaggcggccacaaaggcggcattgccagtggcgcatcacggcgcgcccatacccgtcaacagttcttggagggtctctcatgaccacacgtgaacgtctcccccgcaacggctacagcatcgccgctgctgcgaaaaagctcggtgtctccgagtccaccgtcaagcggtggacttccgagccacgcgaggagttcgtggcccgcgttgccgcacgccacgcgcggattcgtgagctccgctcggagggtcagagcatgcgtgcgattgctgccgaggtcggggtttccgtgggcaccgtgcactacgcgctgaacaagaatcgaactgacgcatgaccgtaacgccgcacgatgagcattttcttgatcgtgcaccgcttggcactacgttcgcgtgcggttgcacagtgcgcgccacgttcttatcctgcggccattgtggctacagccaatggggggcatcagcaacggacgttgaacccggtgggcaagtgttactcagggggacatgcccagtctgcggcgctcggattgacggtatggcagtcgtgcatgcggccccaccgtcaaactcattcaggtatcagtgagaaccctcatggcaccccctcgtgacacgttctcgttgcgatcagctgcggccaagcttgatcttcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgacccgaacatgtcgcgtgggtgcaaaatgagctcaacgaaacaccccgacaaatcctcggcggcgcaaccccacgtgaaatacttaacgaactattcaagcgtggcgcatccaccgcttgattccgccttgaaggcatgttagagtttggggcatgtcgagtcccgaatcagacaggtcaggggttgtcggtcacaccgccagccccttgaaccactagttacgacgcccacgatctgtgtgggcgtatgtctggcgtacccggggcgctggccgtggtgacaagaagaaccatttcttgatctcacacctcggagtactcgatgccttttgccctctacatgcttgccctggcggtcttcgtcatgggcacttcagaattcatgctcgcgggattgctccccgcgatcgcgaccgaacttgacgtctcggtcggcactgcgggcctgctgacctccgcattcgcagtcggtatggtcgtcggcgcgccagtgatggcggcattcgctcgccgttggccaccgcggctcacattgatcgtttgccttctcgtgttcgcgggaagccacgtcatcggagcgatgacaccagtgttctctctcctgctcatcacccgggtgctcagcgctctcgcaaacgcaggattcctcgccgtagcactgagcacggccactaccctcgtgccagcgaaccagaaggggcgtgcactgtcgatcctgctctccggcacgacgatcgcaaccgtcgtgggcgtccccgccggggcactgctcggcacagcgctgggctggcgaacgacgttctgggcgatcgccatcctctgtattcccgcggccgttggagtcattcgtggcgtcacgaacaatgttggtcggagcgagactagcgcgacctcaccaaggctccgtgtcgagctcagccagttggcgacgccgcggctcatcctggccatggcactcggagcgctgatcaacggagggacctttgcggcattcaccttcctggcacccatcgtgaccgagaccgcgggcttggccgaagcgtgggtgtccgtcgcgctggtgatgttcggcatcggatcgttccttggcgtcacgatcgcaggacgactatcagatcaacgacctggcctcgtgctcgcagtcggcggaccgctattgctgacaggctggatcgtgttggcagtggtcgcatctcatcccgttgcgcttatcgtcctcgtcctcgttcagggattcctgtcgttcggcgtcggcagtactctgatcacgcgtgtgctgtatgcagcatcgggtgcgccaacgatgggcggttcgtacgcaaccgcagcattgaatatcggagctgcagcggggcccgtgcttggtgcgctcgggctcgcgaccgggctggggctgctcgcgccggtttgggtcgcttcggtgctgacagcgatcgctctcgtcatcatgcttctcaccagacgcgcgcttacgaagaccgcggcggaggccaattgcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 17 - protein, 663 aa, synthetic construct
MTAQRLQLTWYNKDKALIPTETGKYGYTWVDPSDPRYCETHTLVLDDYVQGSQTPKSDEFAYSERADLEPQDDNLLILGESGDVLEALTRVPELAEKYVGKVKLIYIDPPFNTAQTFASYEDNLEHSIWLTMMRDRLHHMKKLLADDGSIWVHLDYAENHRMRLLLDEVFGCSNFIAEFVWQKADSPRGDAQRVSVDQDVILCYAASGSTVMNRMERTAADNARFSNPDGDSKGVWFSDNRSAPTNVMSWQHPSTFAIQHPISGEMIYPAKGGCWRFGRERLLESLNEYAEYASGDVDIAARVANTSLRSDQVRSDIPDLVLVDPASAAQCARTRIDDGNWPEFFVTATSFGRKSYPPNEGQPARSWWPNDQVGHNREAKSEIKALFSGATPFSTPKPERLLERIIHIGSNPGDIVLDVFAGSGTTAAVAQKMGRRWVTCELLESTFTTFTRPRLEKVLNDQDPGGITRTKGERVDATEDGLPDGVSPEDAAKFTSVLNKLIKDDPELKKSIEVKTLKAASKTRRTKEVVNWRGGGGFQVAHLSPACFDYAPELDRVMLTAAATGQTLIESVTANLGFTLLHPDDDYIFDARRGNALLKVVEGVATTEIVDWLASQIQPGETIVLAATTVMDGVRQHLRKLVKGSRVVALPDDVFRYSEGGDQ

SEQ ID NO: 18 - protein, 31 aa, synthetic construct
MWPIRTVRAYCAGTVATAVALAAFVTSGAHA

SEQ ID NO: 19 - protein, 25 aa, synthetic construct
MIRSLVATLAVGAIVTAGAQMPAQA

SEQ ID NO: 20 - protein, 27 aa, synthetic construct
MLRRAVVGAVTAAVTSTTVLSVVPAGA

SEQ ID NO: 21 - protein, 28 aa, synthetic construct
MALRRTLTLMLCAITGASLVATAVPSSA

SEQ ID NO: 22 - protein, 30 aa, synthetic construct
MYHHSWHSPDARRRGVTRWATTFIAALTAA

SEQ ID NO: 23 - protein, 44 aa, synthetic construct
MEHRYGASQVSGSAPRRGRGVAFAAITGAILLGTVASVDPGAQA

SEQ ID NO: 24 - protein, 48 aa, synthetic construct
MMPENEDELTRSVDKAARNTLGRRTFLGGVTLVAAAAVTGTTPALAHA

SEQ ID NO: 25 - protein, 33 aa, synthetic construct
MNCSFNRRQRSWLAAMVSGMTVITLVPAAVAHA

SEQ ID NO: 26 - protein, 32 aa, synthetic construct
MKQRFKSPTTAVLTGVLIAGSALLLPPLPAKA

SEQ ID NO: 27 - protein, 29 aa, synthetic construct
MNHRRRARIAGLSVLCMAAGSFVAKPAFA

SEQ ID NO: 28 - protein, 26 aa, synthetic construct
MKINARFAVMAASVAVLMAAAPIAQA

SEQ ID NO: 29 - protein, 29 aa, synthetic construct
MRRKSALGFVALSLFATGMGVAAATPATA

SEQ ID NO: 30 - protein, 30 aa, synthetic construct
MTFTKKLSALAIAGTMAITGASIATVPAVA

SEQ ID NO: 31 - protein, 36 aa, synthetic construct
MTNTSTNAGTTLKRTVALAAAASLAVMGTIAEEAHA

SEQ ID NO: 32 - protein, 26 aa, synthetic construct
MSKVVASAIAGALSLTSAGGLTMVQA

SEQ ID NO: 33 - protein, 53 aa, synthetic construct
MRTDLLRLVSLSRWHNSRREHNMRRSTVKRRTVMAAAAGAFAASALPLSRAAA

SEQ ID NO: 34 - DNA, 52 bp, synthetic construct
  source 1..52: mol_type = other DNA
tcgtcggcagcgtcagatgtgtataagagacagttgctcgcaactgcaagca

SEQ ID NO: 35 - DNA, 54 bp, synthetic construct
  source 1..54: mol_type = other DNA
gtctcgtgggctcggagatgtgtataagagacagccggctggcaaatgaggcat

SEQ ID NO: 36 - DNA, 50 bp, synthetic construct
  source 1..50: mol_type = other DNA
tcgtcggcagcgtcagatgtgtataagagacagcctacgggnggcwgcag

SEQ ID NO: 37 - DNA, 55 bp, synthetic construct
  source 1..55: mol_type = other DNA
gtctcgtgggctcggagatgtgtataagagacaggactachvgggtatctaatcc

SEQ ID NO: 38-40 - skipped (3 entries)